(19) United States
(12) Patent Application Publication
(10) Pub. No.: US 2012/0190723 A1
(43) Pub. Date: Jul. 26, 2012

(54) VIRAL MODULATORS AND PROCESSES THEREOF

(76) Inventors: Fatah Kashanchi, Potomac, MD (US); Kylene Kehn-Hall, Fredericksburg, VA (US); Olaf Kutsch, Birmingham, AL (US)

(21) Appl. No.: 13/325,464

(22) Filed: Dec. 14, 2011

Related U.S. Application Data
(60) Provisional application No. 61/422,878, filed on Dec. 14, 2010.

(51) Int. Cl. C12N 7/06; A61P 31/18; C12Q 1/68; A61K 31/404; C12N 15/85

(52) U.S. Cl. 514/418; 435/238; 435/455; 435/6.1

(57) ABSTRACT

A viral modulator and process thereof. A method may include contacting one or more viral modulators to one or more biological systems. A biological system may be configured to be infected by one or more virus. A virus may include an HIV virus, a VEEV virus and/or the like. A viral modulator may include a viral inhibitor and/or a viral activator.

### CROSS-REFERENCE TO RELATED APPLICATION

This application claims the benefit of provisional Application No. 61/422,878, filed on Dec. 14, 2010, which is hereby incorporated by reference in their entirety.

### STATEMENT REGARDING FEDERALLY FUNDED RESEARCH

This invention was made with government support under and National Institute of Health Grant Nos. AI0078859, AI0074410, NS070740, NS060632 and NS070740. The government has certain rights in the invention.

### BACKGROUND

Anti-viral therapies, for example RT, PR and fusion inhibitors for Human Immune Deficiency Virus (HIV), may operate to prolong the lifespan of an infected subject. However, viral infection may result in a wide array of neurological disorders that may not be addressed by such therapies. Neurological disorders exhibited by subjects infected with HIV, for example, may include HIV-associated dementia, mild neurocognitive disorder, and asymptomatic neurocognitive impairment that are not prevented with HAART. Collectively, such neurological disorders may be referenced as HIV-associated neurologic disease (HAND). Thus, while anti-viral therapies may prolong the life span of infected subjects, the quality of life of infected subjects may be impacted by neurological disorders.

Anti-viral therapies may not be available to minimize the impact of other viral infections and/or associated disorders. For example, Venezuelan Equine Encephalitis Virus (VEEV) may cause disease in equine and humans. Symptoms of VEEV infection may include malaise, fever, chills, retro-orbital or occipital headache, and central nervous system involvement including convulsions, somnolence, confusion, and photophobia. While VEEV infection in humans may be lethal in a relatively small percent of cases (less than approximately 1%), children may be particularly susceptible. Furthermore, neurological disease, including disorientation, ataxia, mental depression, and convulsions, may occur in up to approximately 14% of infected subjects. Neurological sequalae may also be common. Moreover, VEEV may cause infection by a respiratory route, and may be weaponized. Despite VEEV associated disorders and route of infection in humans and other subjects, there may not be any specific antiviral therapeutics for the treatment of VEEV.

Anti-viral therapies may not be available to maximize and/or leverage viral infection. For example, it may be desired to up-regulate viral replication. Increasing viral load may, for example, facilitate identification and/or targeting of a virus which may result in enhanced therapy, enhanced therapy development and/or the like. Accordingly, there may be a need for compositions and/or processes that may modulate viral replication and/or effects. For example, there may be a need to minimize viral replication, viral-induced neurotoxicity, viral-induced cell death and/or the like. As another example, there may be a need to maximize viral replication, viral-induced neurotoxicity, viral-induced cell death and/or the like.

### SUMMARY

According to some aspects of embodiments, a method may include contacting one or more biological systems with one or more viral modulators. In some aspects of embodiments, a biological system may be configured to be infected by one or more viruses. In another aspect of embodiments, a biological system may be configured to be infected by a virus (e.g., HIV-1). In another aspect of embodiments, a biological system may be configured to be infected by VEEV.

According to some aspects of embodiments, a biological system may include a subject, for example a human subject. In one aspect of embodiments, a subject may include a healthy subject, an infected subject, a subject at risk for an infection and/or the like. In another aspect of embodiments, contacting may include administering a therapeutically effective amount of one or more viral inhibitor compounds (e.g., inhibitor) to a subject. In further aspects of embodiments, a biological system may include an in vitro system. In more aspects of embodiments, an in vitro system may include an assay system and/or a screen.

According to some aspects of embodiments, a cell may be transduced with an expression vector. In one aspect of embodiments, for example, a JLTRG cell may be transduced with a Tat expression vector. In another aspect of embodiments, an expression vector may be under the control of a promoter, for example a Tat expression vector configured to be under the control of a murine stem cell promoter. In further aspects of embodiments, a selected cell may be isolated from transduced cells, for example isolating an eGFP-expressing cell from JLTRG transduced cells.

According to some aspects of embodiments, one or more other additional transductions on an isolated expressing cell may be performed employing an expression vector, for example on an isolated eGFP-expressing cell employing a Tat expression vector configured to be under the control of a murine stem cell promoter. In one aspect of embodiments, an isolated expressing cell may be further transduced with one or more other expression vectors, for example transducing an isolated eGFP-expressing cell employing an RFP-expressing vector. In another aspect of embodiments, single cell cloning may be performed for expressing cells to isolate assay and/or screen cells, for example single cell cloning may be performed for eGFP/RFP expressing cells to isolate a TiGR cell.

According to some aspects of embodiments, an HIV viral inhibitor may include 6-bromo-5-methyl-1H-indole-2,3-dione 3-[(6-bromo-5-methyl-2-oxo-1,2-dihydro-3H-indol-3-ylidene)hydrazone], which may exhibit an IC₅₀ of less than approximately 30 nM, for example between approximately 0.03 nM and 0.5 nM. In other aspects of embodiments, an HIV viral inhibitor may include 4-bromo-5-methyl-1H-indole-2,3-dione 3-oxime and/or 6-chloro-7-methyl-1H-indole-2,3-dione 3-oxime. In embodiments, an HIV viral inhibitor may be employed to minimize viral infection, neurological disease, for example minimize HAND, and/or the like.

According to some aspects of embodiments, an HIV viral activator may include N′-(5-bromo-2-oxo-1,2-dihydro-3H-indol-3-ylidene)-2,4-dichlorobenzohydrazide and/or N-(2-{[2-(5-bromo-2-oxo-1,2-dihydro-3H-indol-3-ylidene)hydrazino]carbonyl}phenyl) benzamide. In some aspects of embodiments, an assay system and/or a screen system may be contacted with one or more viral activator, for example to target infection.

According to some aspects of embodiments, a VEEV viral inhibitor may include 6-bromo-5-methyl-1H-indole-2,3-dione 3-[(6-bromo-5-methyl-2-oxo-1,2-dihydro-3H-indol-3-ylidene)hydrazone], which may exhibit an IC₅₀ of approximately 0.5 μM and/or a CC₅₀ of greater than approximately 100 μM. In other aspects of embodiments, a VEEV viral inhibitor may include N′-(5-bromo-2-oxo-1,2-dihydro-3H-indol-3-ylidene)-2-chlorobenzohydrazide, N′-(5-bromo-2-oxo-1,2-dihydro-3H-indol-3-ylidene)-2,4-dichlorobenzohydrazide,5,7dibromo-1H-indole-2,3-dione 3-oxime and/or 4-chloro-7-methyl-1H-indole-2,3-dione 3-oxime. In embodiments, a VEEV viral inhibitor may be employed to minimize viral infection, neurological disease, for example minimize VEEV-related diseases, and/or the like.

### DRAWINGS

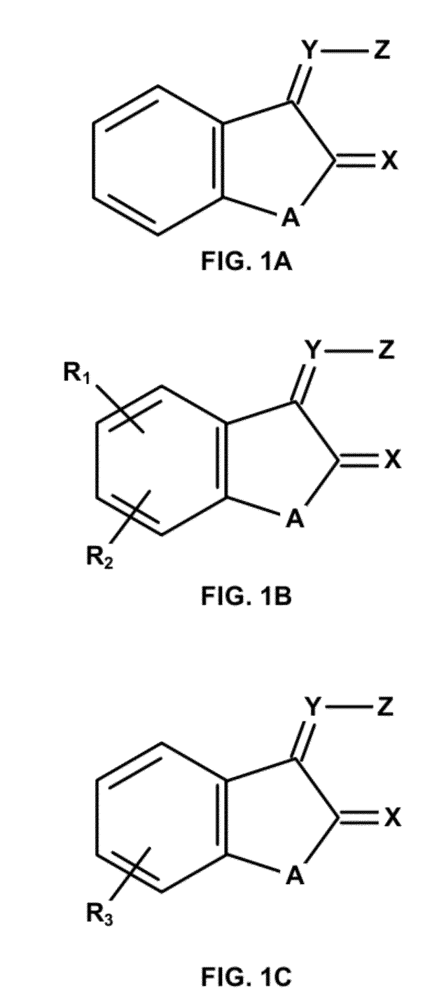

Examples FIG. 1A to FIG. 1C illustrate example viral modulators in accordance with some aspects of embodiments.

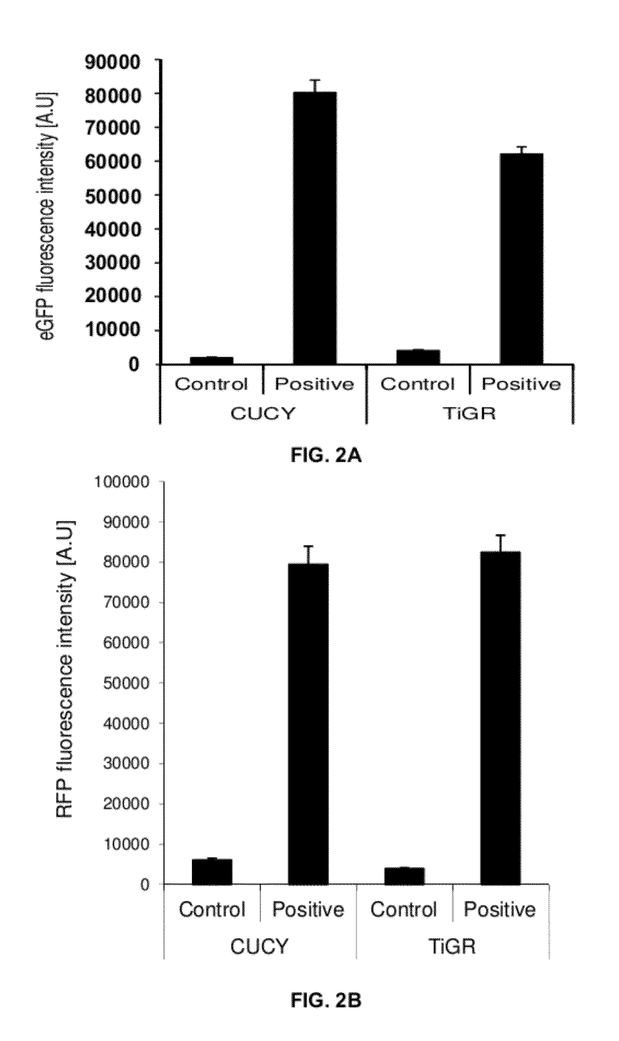

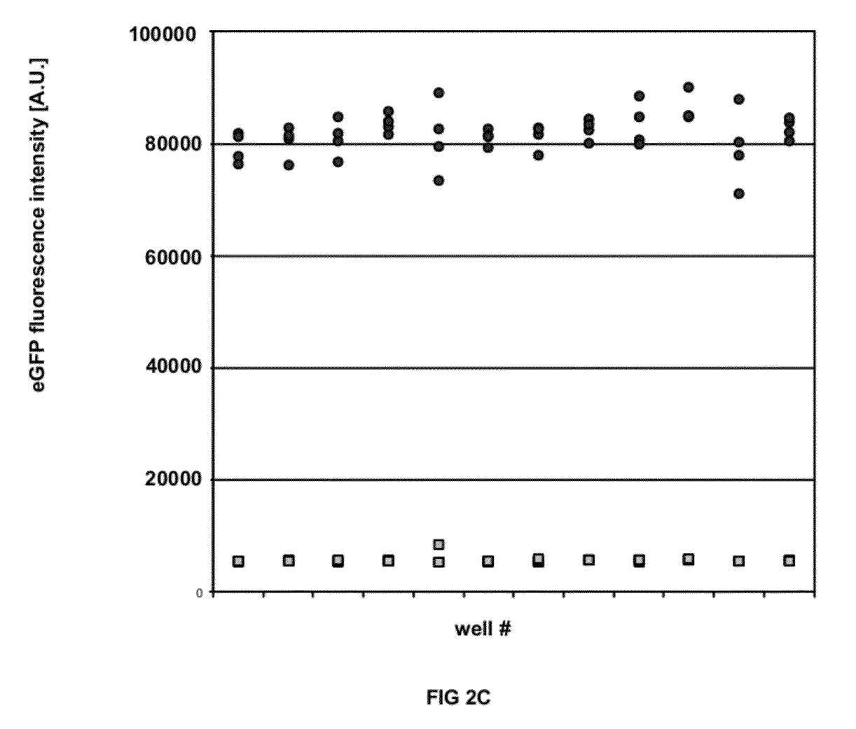

Examples FIG. 2A to FIG. 2C illustrate an HIV-1 transcriptional inhibitor screening system in accordance with some aspects of embodiments. Comparison of eGFP (A) and RFP (B) expression in CUCY cells and TIGR reporter cells employed. (C) Z-test results for TiGR cells. Approximately 2×10⁵ TiGR cells may be loaded into 48 wells of a 384-well plate and eGFP fluorescence intensity may be measured (black circles) compared to eGFP fluorescence of the parental JLTRG cells (gray squares) loaded into 48 wells of the same plate.

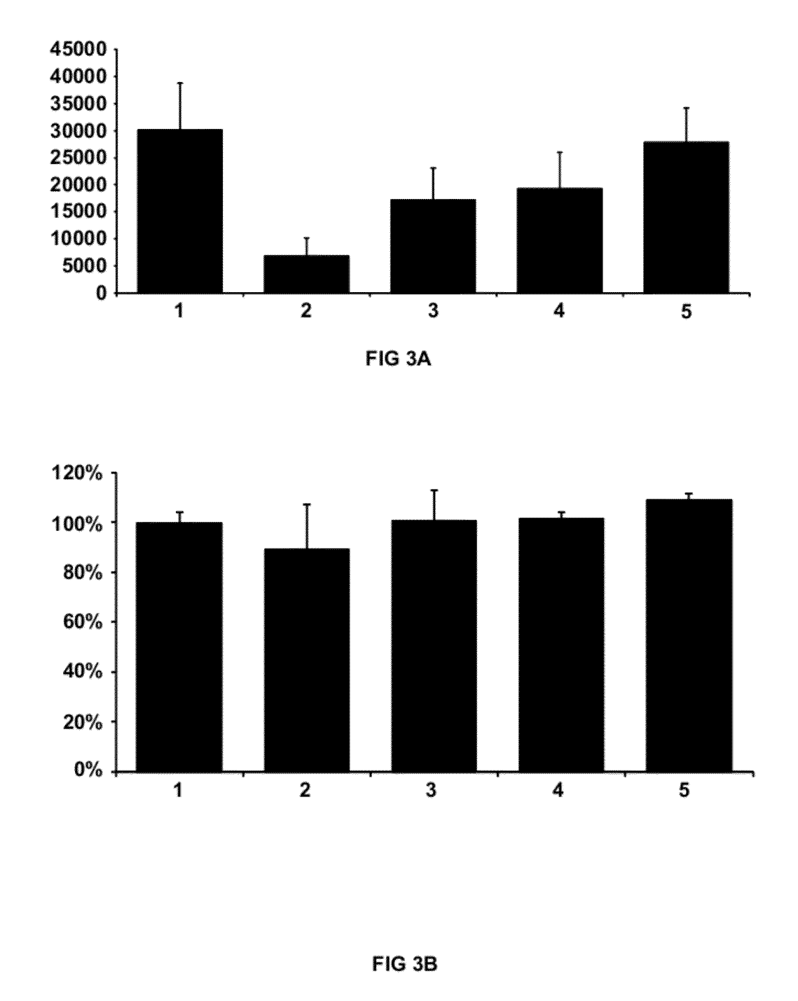

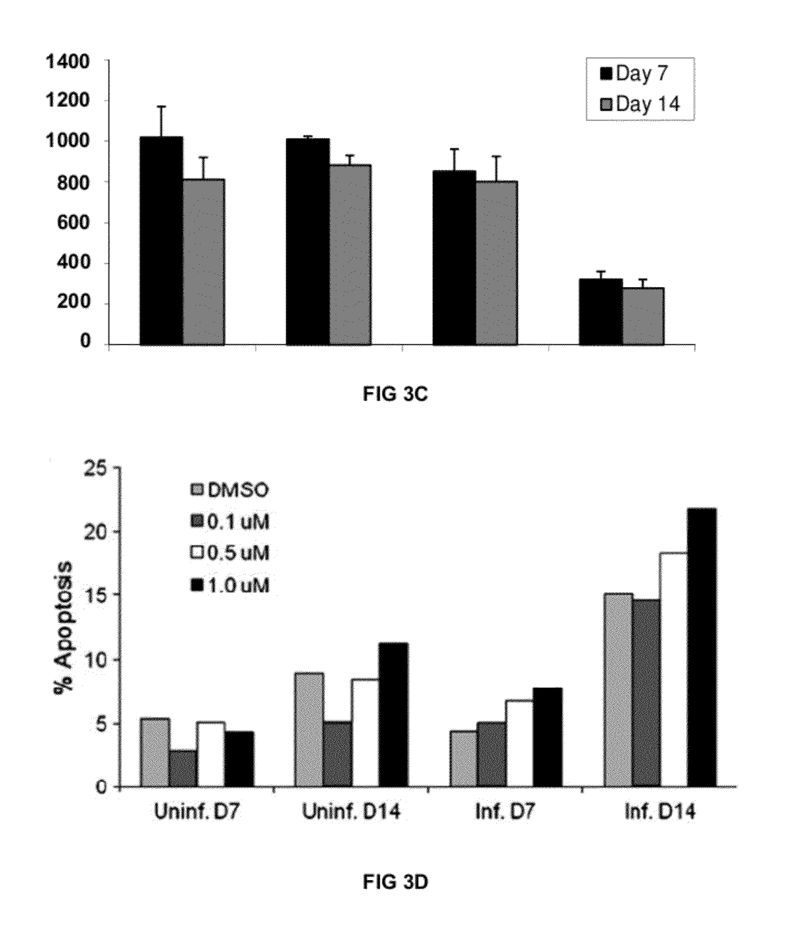

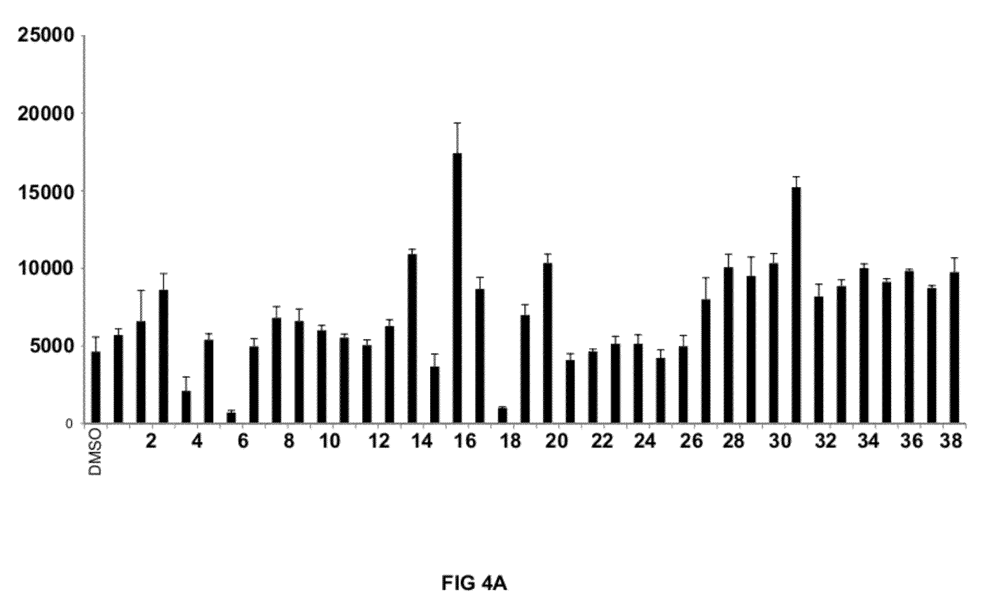

Examples FIG. 3A to FIG. 3D illustrate that BIO may inhibit HIV-1 transcription and/or replication without substantially inducing cellular toxicity in accordance with some aspects of embodiments. (A) TZM-bl cells may be transfected with approximately 1.0 μg of Tat and treated the next day with DMSO and/or BIO (approximately 0.025, 0.05, 0.1, and 1.0 μM). Cells may be processed 48 hours post drug treatment for luciferase assays. Assays may be performed in triplicate and an average value is shown plus standard deviation. (B) TZM-bl cells may be treated with DMSO and/or BIO (approximately 0.025, 0.05, 0.1, and 1.0 μM). Cell proliferation/viability may be determined by MTT assays. Treatments may be performed in triplicate and samples analyzed at 48 hours. (C) PHA and IL-2 activated PBMCs may be kept in culture for approximately 2 days prior to infection. Isolation and treatment of PBMCs may be performed by following the guidelines of the Centers for Disease Control. Approximately 2.5×10⁷ PBMCs may be infected with 89.6 (approximately 35,520 RT units). Cells may be resuspended in approximately 6.5 ml of complete media and plated in a 96 well plate at approximately 200 μl/well. BIO treatment (approximately 0, 0.1, 0.5 and 1.0 μM) may be performed (only once) the day following infection. Samples may be collected on days 7 and 14 and stored at approximately −20° C. for RT assay. Treatments may be performed in triplicate and the average plus the standard deviation is displayed. (D) PBMCs may be processed as described for (C). Cells may be collected on days 7 and 14, washed 2× with PBS without Ca and Mg, resuspended in approximately 70% ethanol, and stained with propidium iodine prior to cell cycle analysis by flow cytometry to determine apoptosis (sub-G1 peak). Triplicate wells were pooled for this analysis.

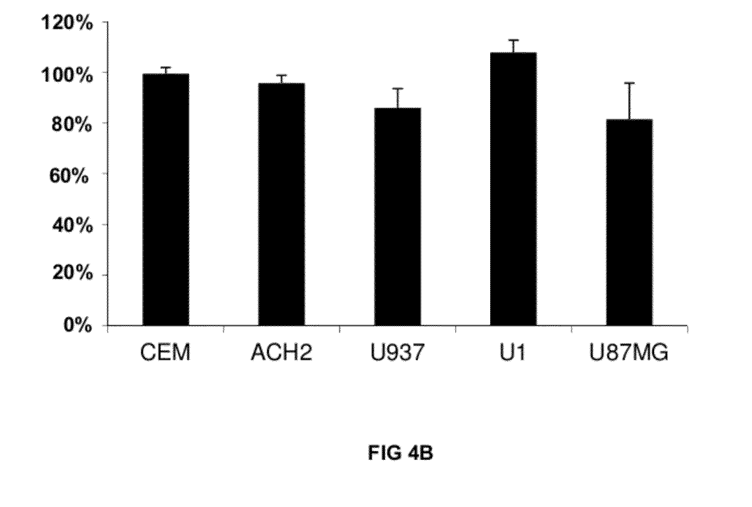

Examples FIG. 4A to FIG. 4B illustrate BIO analogs may modulate HIV-1 transcription without substantially inducing cellular toxicity in accordance with some aspects of embodiments. (A) TZM-bl cells may be transfected with approximately 1.0 μg of Tat and treated the next day with DMSO, BIO, analog compounds I-38 at approximately 1 μM. Cells may be processed 48 hours post drug treatment for luciferase assays. Assays may be performed in triplicate and an average value is shown plus standard deviation. (B) CEM, ACH2, U937, U1, and U87MG cells may be treated with DMSO and compound 6 (BIOder) (approximately 1 μM). Cell proliferation/viability may be determined by MTT assays. Treatments may be performed in triplicate and samples analyzed at 48 hours. Percent viability is expressed as compared to the DMSO control.

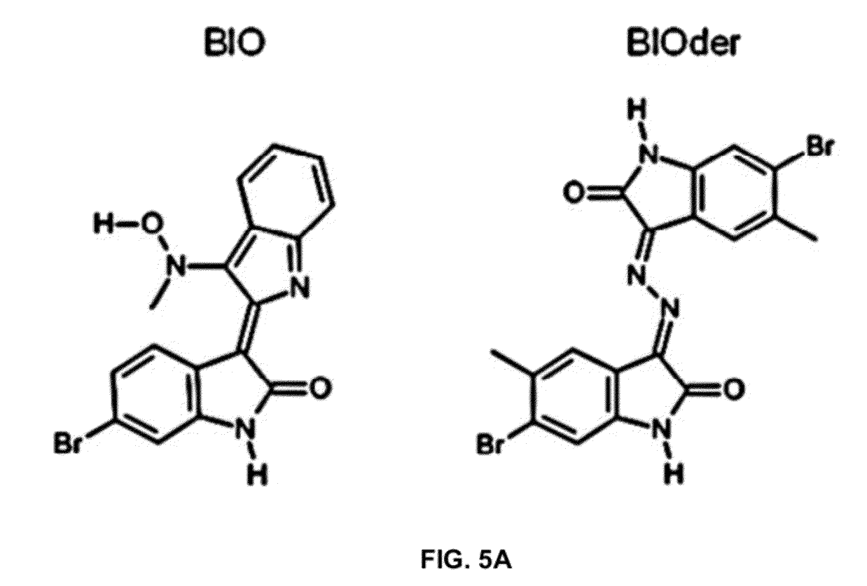

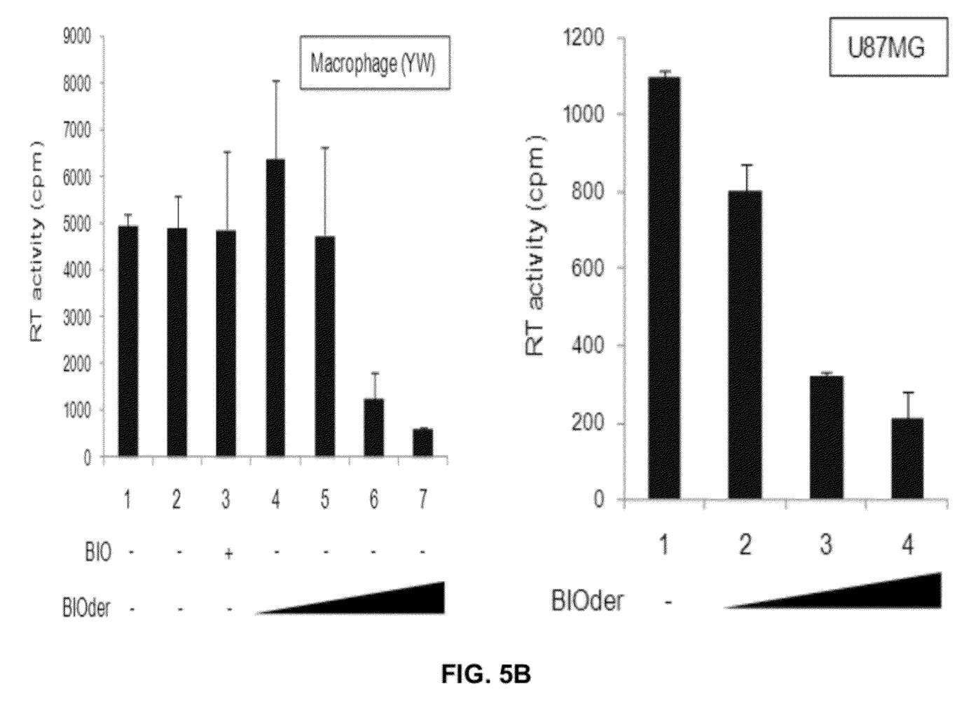

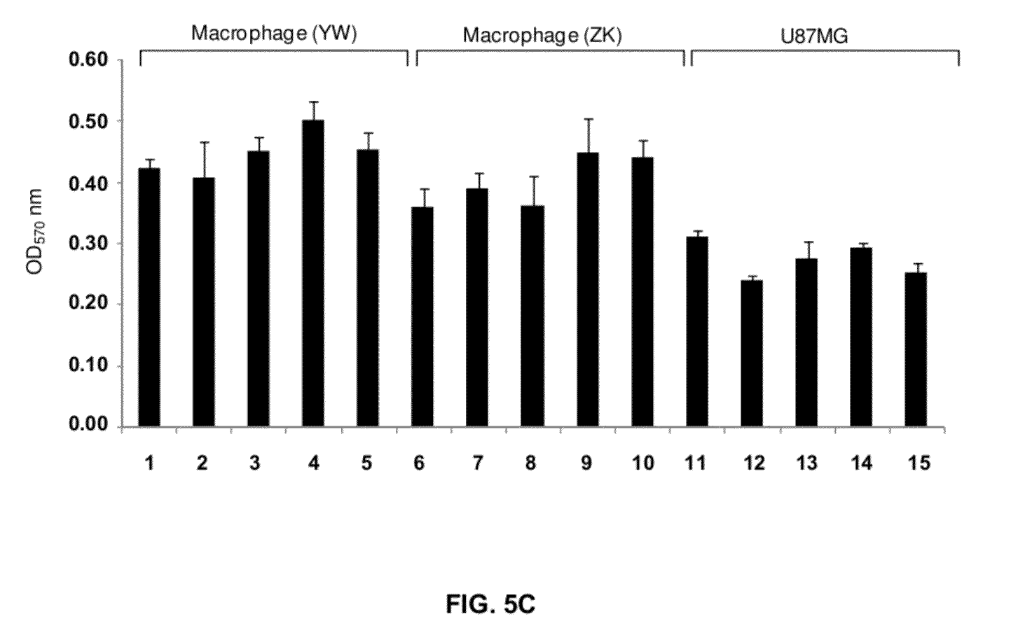

Examples FIG. 5A to FIG. 5C illustrate an effect of BIO and/or BIOder on HIV-1 replication in accordance with some aspects of embodiments. (A) Structure of BIO: 2-[[[2-(5-bromo-2-oxo-1,2-dihydro-3H-indol-3ylidene)hydrazino](oxo)acetyl]amino]benzoic acid and BIOder: 6-bromo-5-methyl-1H-indole-2,3-dione 3-[(6-bromo-5-methyl-2-oxo-1,2-dihydro-3H-indol-3-ylidene)hydrazone]. (B) PMBCs may be obtained from the blood of healthy donor (YW), and purified by centrifugation through a layer of Lymphocyte Isolation Medium. Cells may be re-suspended in serum-free RPMI and plated on culture dish for approximately 1 hour at approximately 37° C. Non-adherent lymphocytes may be removed and the adherent monocytes may be cultured in RPMI plus approximately 10% heat-inactivated FBS. Macrophages may be further differentiated by incubating in approximately 10 ng/ml M-CSF for approximately 1 week with medium change every approximate 2 days. Macrophages and U87MG cells may be infected with 89.6 (MOI: approximately 1). BIO (e.g., lane 3, approximately 10 nM), BIOder (lanes 4-7: approximately 0.1, 1, 10, 100 nM) may be added to cells approximately 6 hours after infection. U87MG cells may be treated with BIOder at approximately 0.1, 1 and 10 nM. Samples may be collected on day 7 for RT assay. Treatments may be performed in triplicate and the average plus the standard deviation is displayed. (C) MTT assays in two monocyte/macrophage healthy donors and U87MG may be performed after treatment with BIOder at approximately 10, 100, 1000 and 10,000 nM (e.g., lanes 2-5).

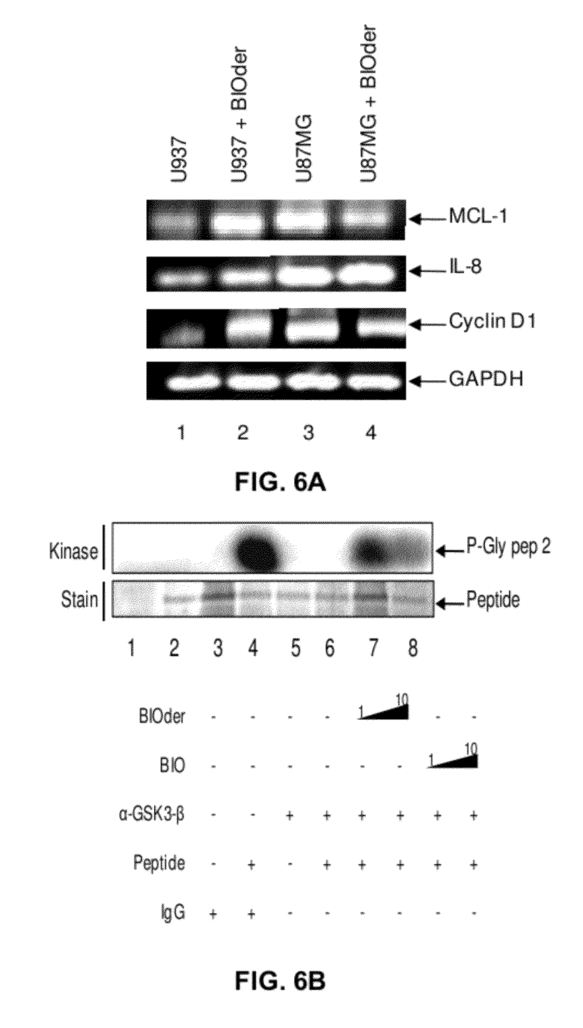

Examples FIG. 6A to FIG. 6B illustrate BIOder may not inhibit cellular gene expression in the absence of Tat and/or may be specific to GSK-3β in accordance with some aspects of embodiments. (A) Total RNA may be isolated from cells treated with 6BIOder (approximately 1 μM) using Trizol. RNA may be treated with approximately 0.25 mg/ml DNase I for approximately 1 hour, followed by heat inactivation at approximately 65° C. for approximately 15 minutes. A total of approximately 1 μg of total RNA may be used to generate cDNA with the iScript cDNA Synthesis kit using oligo-dT reverse primers. Primers for PCR may be MCL-1, IL-8, cyclin D1 and GAPDH as control. (B) Approximately two milligrams of U937 extract may be IPed at approximately 4° C. overnight with GSK-3β antibody. The next day, complexes may be precipitated with A/G beads for approximately 2 hours at approximately 4° C. IPs may be washed twice with TNE buffer and kinase buffer. Phosphorylation reactions may be performed with IP material and approximately 200 ng of glycogen synthase peptide 2 (Millipore) as substrate. Following incubation, samples may be run on an approximate 4-20% SDS-PAGE, dried and subjected to analyzed using Molecular Dynamics Phosphor Imager software.

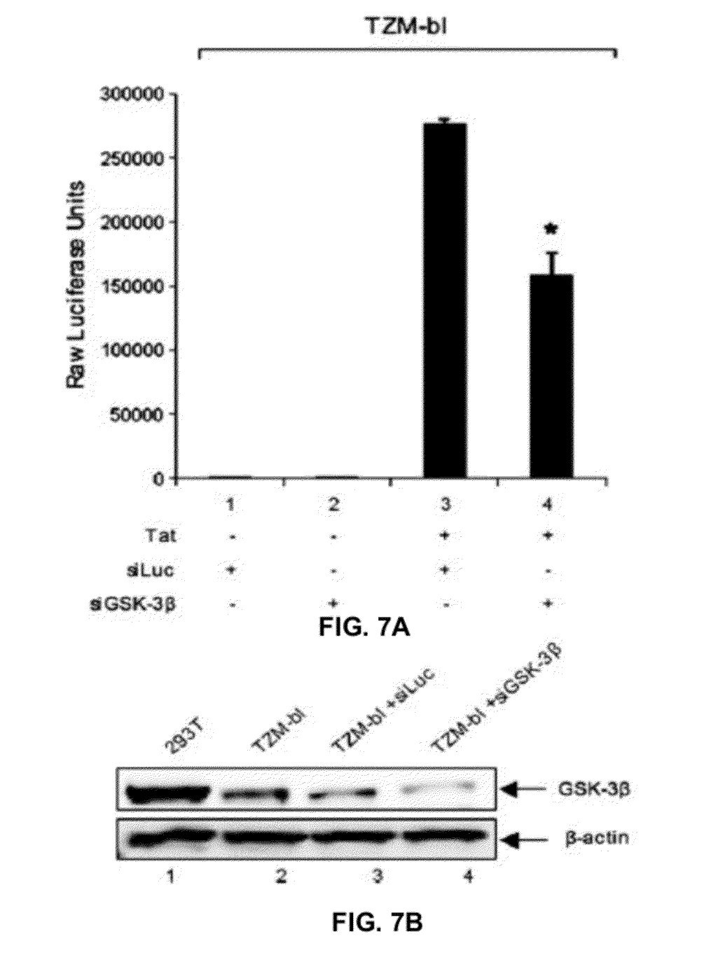

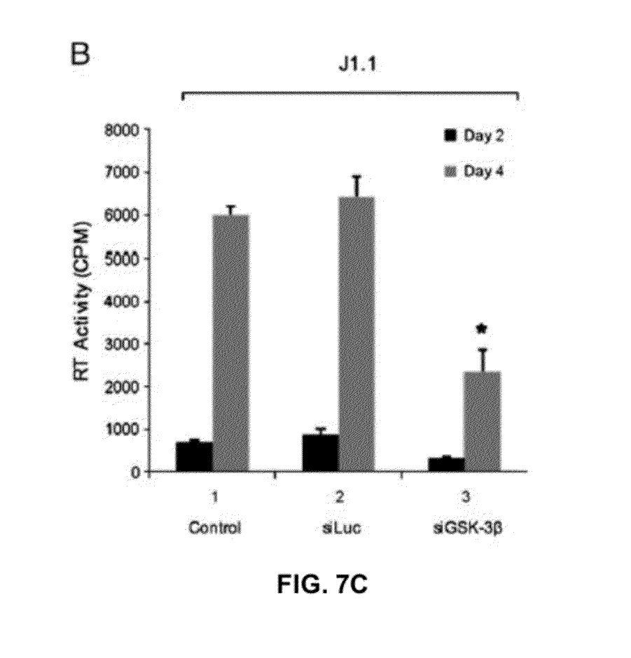

Examples FIG. 7A to FIG. 7C illustrate down-regulation of GSK-3β may relatively decrease viral transcription in cells in accordance with some aspects of embodiments. (A) TZM-bl cells may be transfected with siRNA against GSK-3β or luciferase (approximately 100 nM) in the presence or absence of Tat (approximately 1 μg) and assayed for luciferase expression approximately 48 hours post-transfection. To confirm knockdown, 5 approximately 0 μg of whole cell extract of 293T cells (positive control), TZM-bl, TZM-bl transfected with luciferase siRNA (siLuc), and TZM-bl transfected with GSK-3β (siGSK-3β), may be run on an approximate 4-20% SDS-PAGE and Western blotted against GSK-3β and β-actin. *pb0.01 is related to the comparison between siLuc and siGSK-3β. (B) J1-1 cells were used for electroporation with siLuc or siGSK-3β. Log phase growing cells (approximately 5×10⁵/ml) may be electroporated with either siLuc or siGSK-3β(approximately 200 nm) and re-plated in complete media. Supernatants may be processed for presence of RT at approximately days 2 and 4. The effect of siGSK-3β treatment may be abolished by day 6. *pb0.01 may be related to the comparison between siLuc and siGSK-3β.

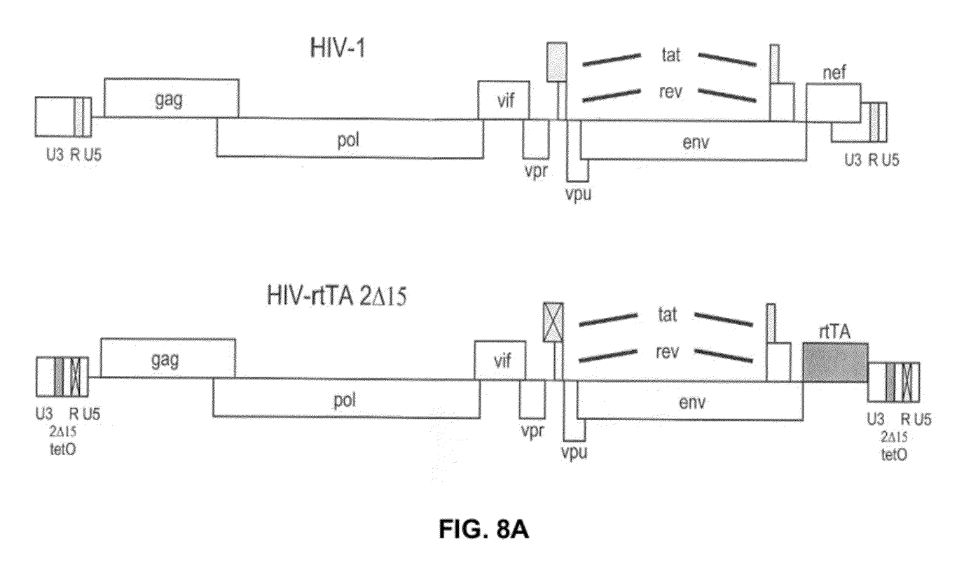

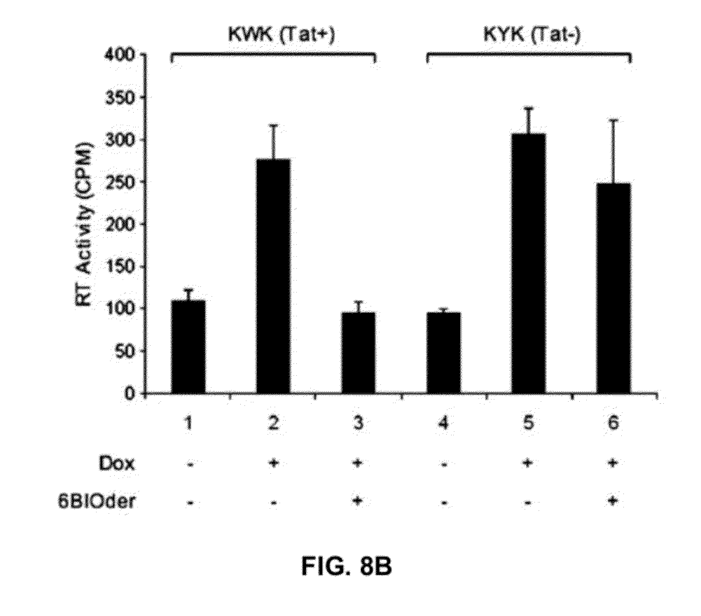

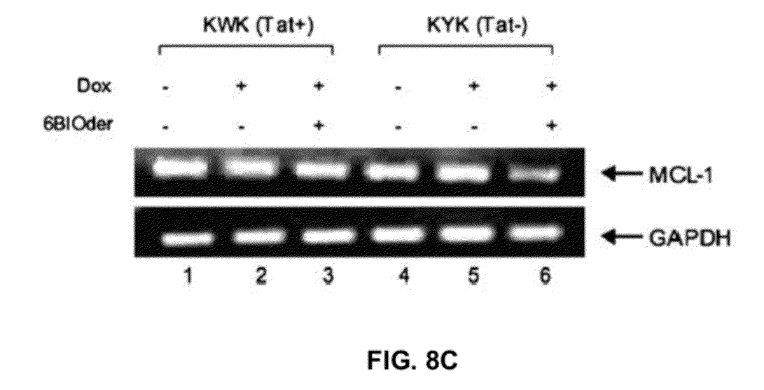

Examples FIG. 8A to FIG. 8C illustrate the effect of BIOder in dox-dependent HIV-1 variants in macrophages in accordance with some aspects of embodiments. (A) HIV-1 genome and modifications may be introduced to construct HIV-rtTA. In brief, TAR-Tat transcriptional axis may be replaced by the tetracycline-inducible tetO-rtTA system. Inactivation of TAR and Tat may be indicated by crosses through the motifs. (B) The pLAI chimera plasmids, KWK and KYK (approximately 20 μg), may be individually transfected (electroporation) into the differentiated macrophages (approximately 4×10⁶) employing approximately 250 nM PMA for approximately 3 days. The culture may be maintained with dox (approximately 1000 ng/ml) and BIOder (approximately 50 nM), and virus replication may be monitored by measuring the amount of RT produced in the culture medium at day 10. No virus replication may be observed in the absence of dox, indicating that replication may be strictly dependent on the inserted Tet system. (C) Macrophages may be treated and transfected as described above in (B). On day 10, cell pellets may be lysed in Buffer RLT and RNA extracted by Qiagen's RNeasy kit. RNA may be treated with approximately 0.25 mg/ml DNase I at approximately 37° C. for approximately 1 hour, followed by heat inactivation at approximately 65° C. for approximately 15 minutes. A total of approximately 30 ng of total RNA may be used to generate cDNA with the iScript cDNA Synthesis kit using random primers. PCR may be performed with GAPDH and MCl-1 specific primers.

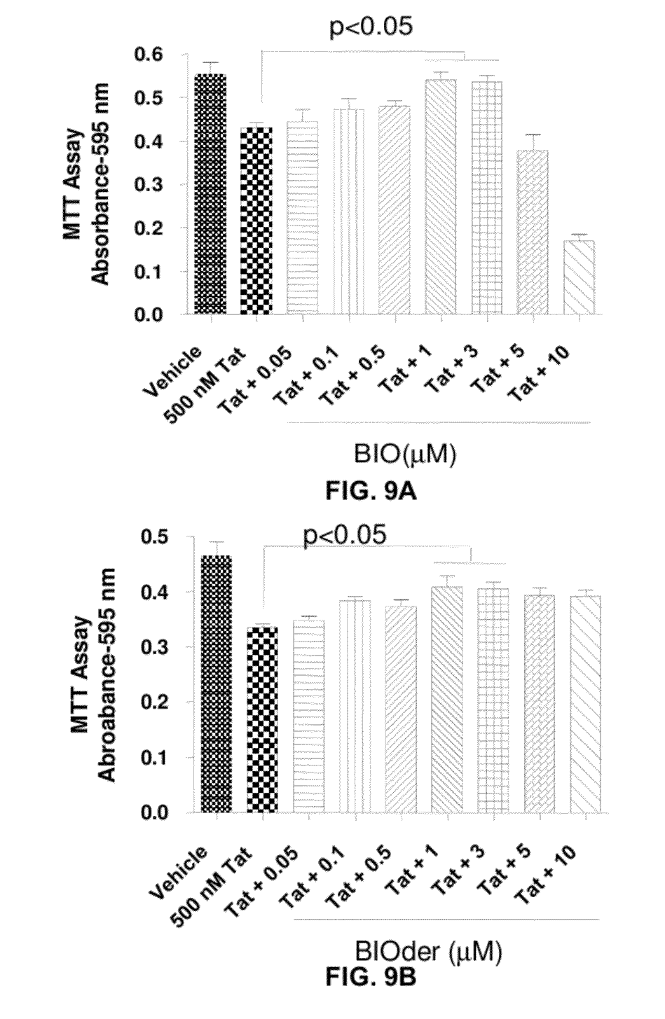

Examples FIG. 9A to FIG. 9B illustrate BIO and BIOder may protect neuronal cultures from the HIV-1 Tat protein in accordance with some aspects of embodiments. Rat mixed hippocampal cultures may be preincubated with various concentrations of (A) BIO (approximately 0.05-10 μM) and/or (B) 6BIOder (approximately 0.05-10 μM) for approximately 1 hour at approximately 37° C. prior to an approximate 18-hour exposure to approximately 500 nM Tat1-72. After approximately 18 hours, cell survival may be measured by MTT assay. Statistical significance may be determined by ANOVA, followed by Newman-Keuls post hoc pair-wise comparisons.

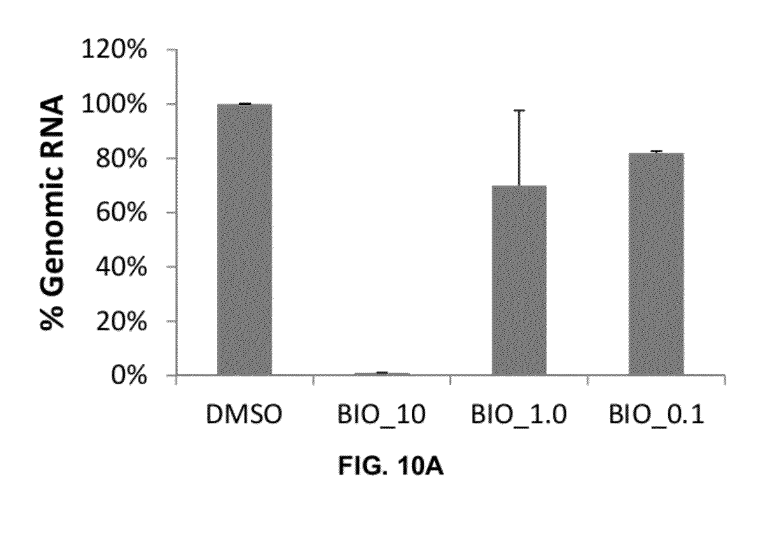

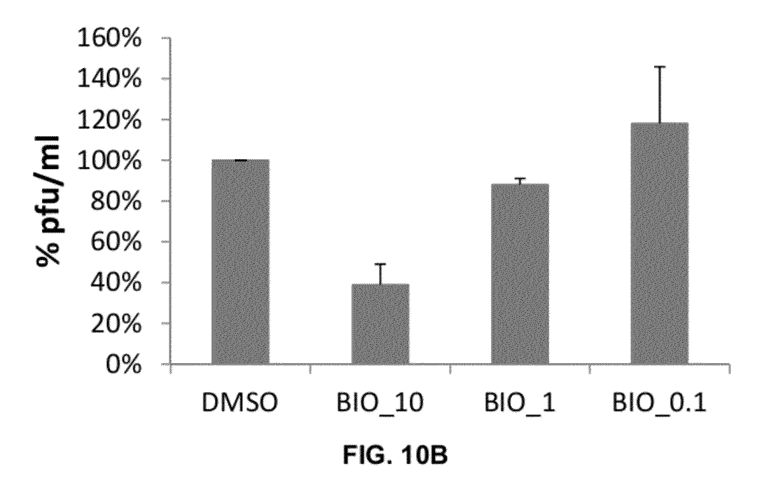

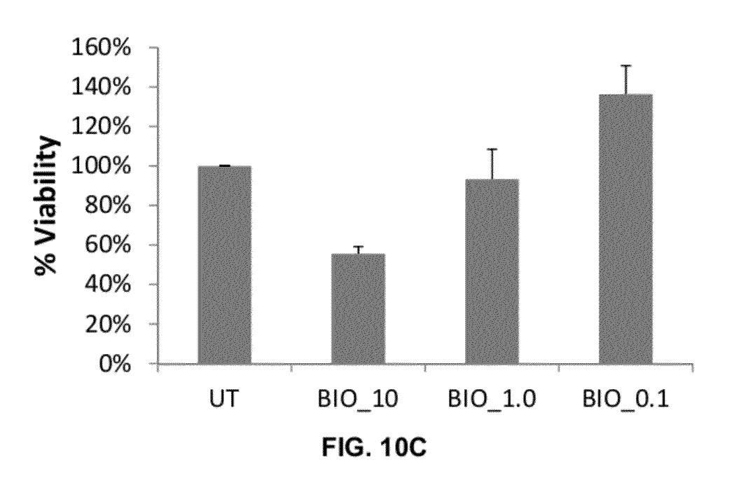

Examples FIG. 10A to FIG. 10C illustrate BIO may inhibit VEEV replication in accordance with some aspects of embodiments. (A) U87MG astrocytes may be pretreated for approximately 2 hours with DMSO, or BIO (approximately 0.1, 1.0, or 10 μM), infected with VEEV TC-83 at MOI approximately 0.1, and post-treated with compounds. Twenty-four hours post infection viral supernatants may be collected and assayed for viral replication by q-RT-PCR. (B) Cells may be treated as in panel A and viral replication measured by plaque assays. (C) U87MG astrocytes may be treated with BIO (approximately 0.1, 1.0, or 10 μM) and cell viability assayed approximately 24 hours later by MTT assays. UT (untreated) cells may be displayed at approximately 100% viability and all treatments compared to those values.

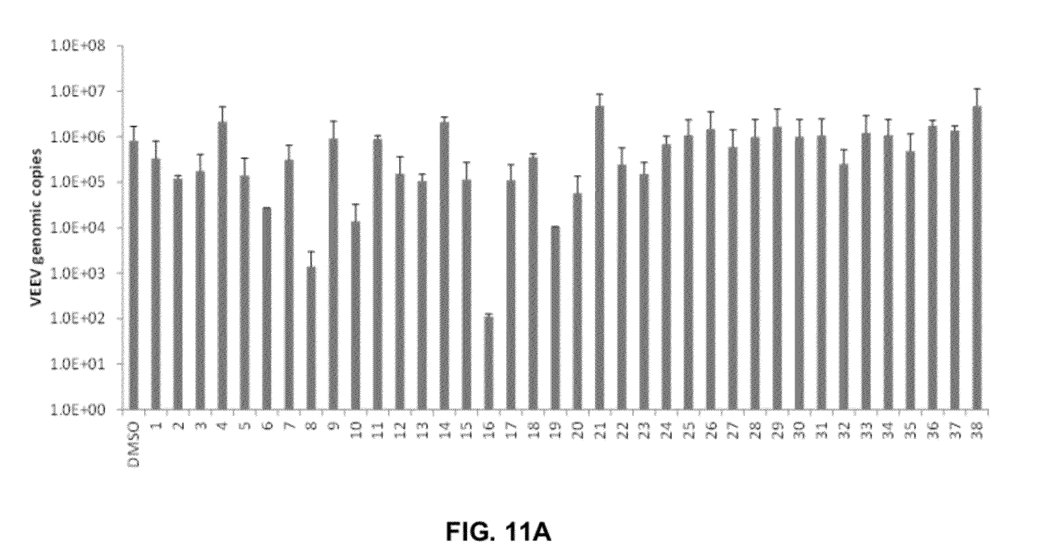

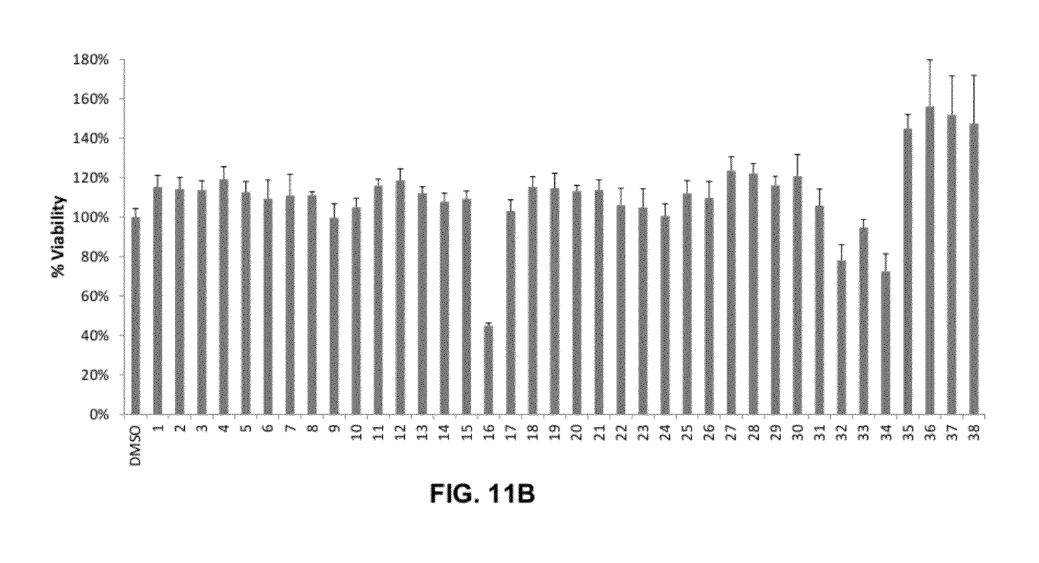

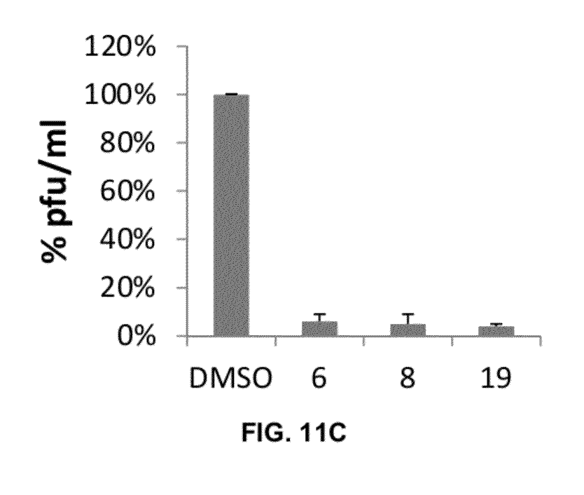

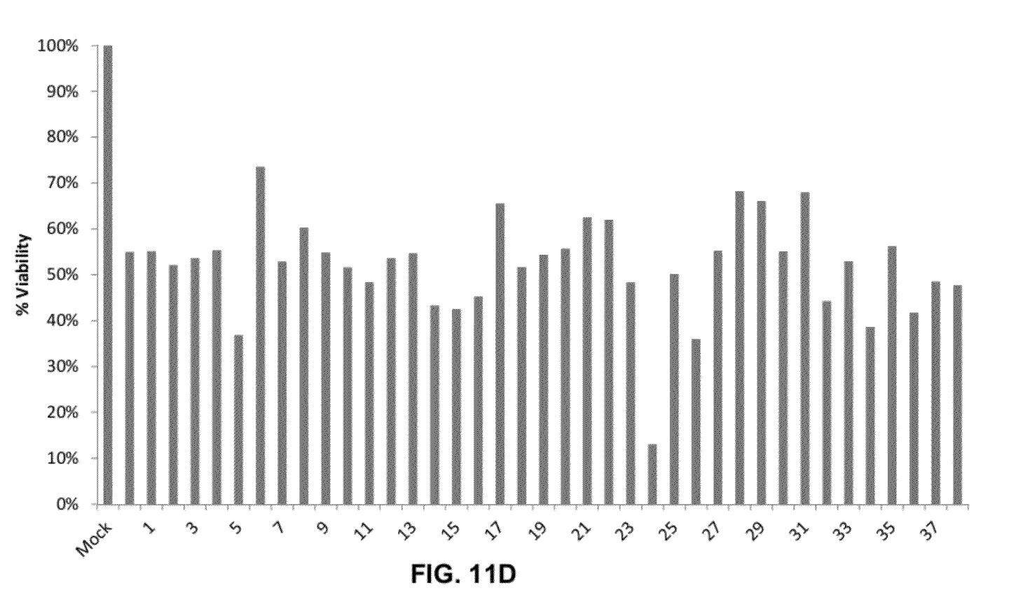

Examples FIG. 11A to FIG. 11D illustrate identification of a compound that may inhibit VEEV replication and/or CPE in accordance with some aspects of embodiments. (A) U87MG astrocytes may be pretreated for approximately 2 hours with DMSO or various BIO derivatives at approximately 1 μM, infected with VEEV TC-83 at MOI approximately 0.1, and post-treated with compounds. Twenty-four hours post infection viral supernatants may be collected and assayed for viral replication by q-RT-PCR. (B) Thirty-eight BIO analogs may be assayed for their ability to inhibit VEEV induced CPE. U87MG astrocytes may be pretreated for approximately 2 hours with DMSO or various BIO derivatives at approximately 1 μM, infected with VEEV TC-83 at MOI approximately 0.1, and post-treated with compounds. Forty-eight hours post infection CPE may be measured by MTT assay. Mock infected cells may display at approximately 100% viability. (C) U87MG astrocytes may be treated with DMSO or various BIO analogs at approximately 1 μM and cell viability assayed. Forty-eight hours post infection CPE was measured by CellTiter Glo luminescence cell viability assay. Mock infected cells may be displayed at approximately 100% viability. (D) U87MG astrocytes may be pretreated for approximately 2 hours with DMSO, BIO, compounds 6, 8, and/or 19 at approximately 1 μM, infected with VEEV TC-83 at MOI approximately 0.1, and post-treated with compounds. Twenty-four hours post infection viral supernatants may be collected and assayed for viral replication by plaque assays.

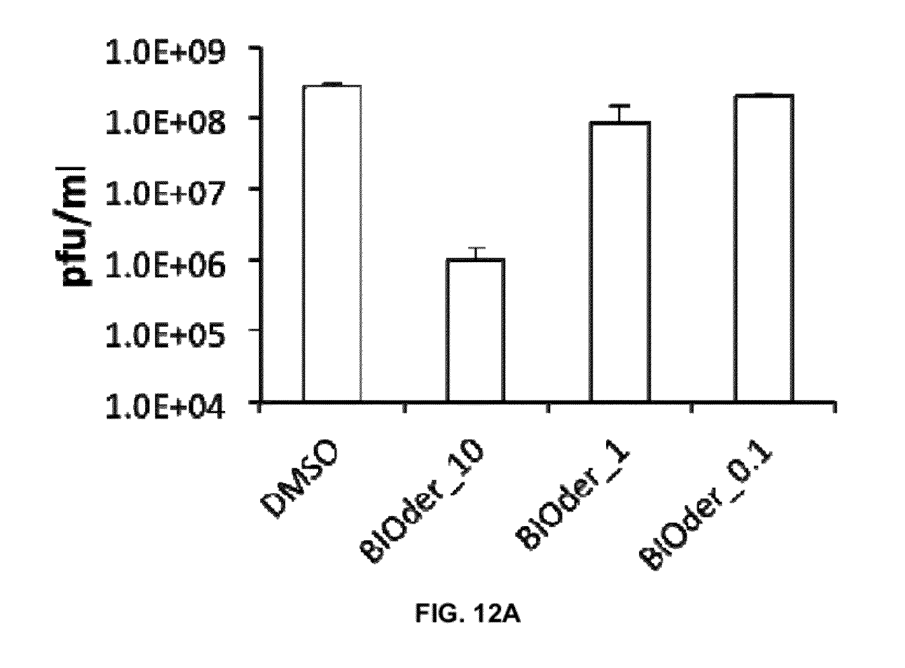

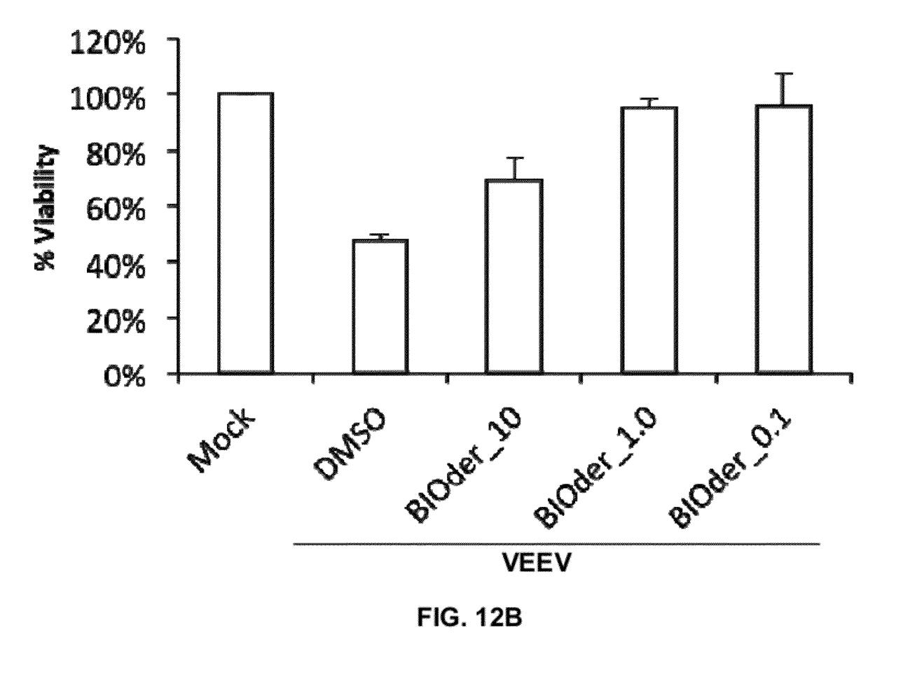

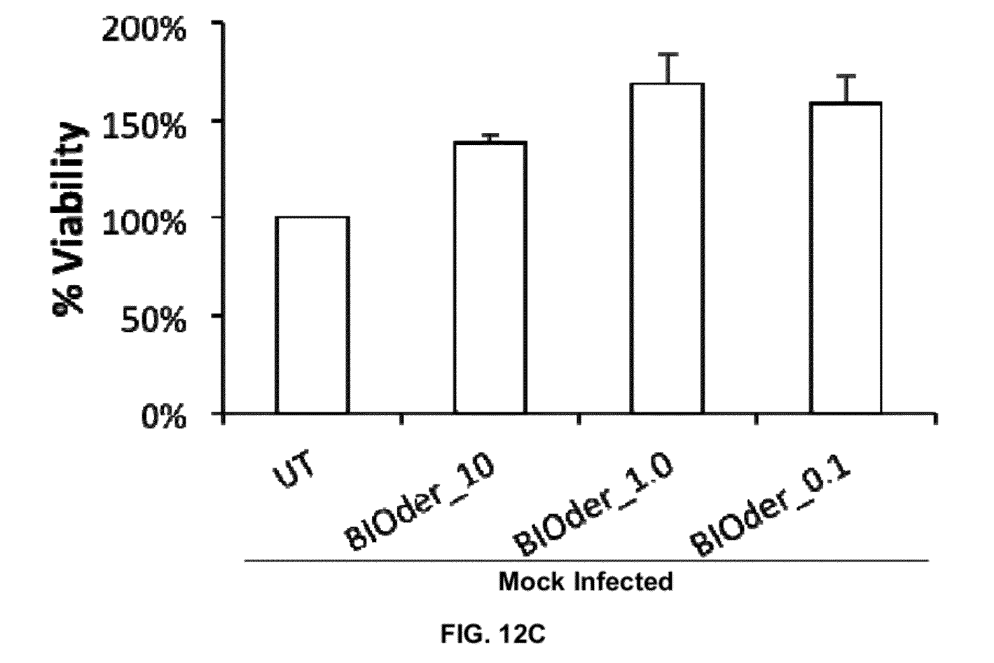

Examples FIG. 12A to FIG. 12C illustrate characterization of BIOder for VEEV in accordance with some aspect of embodiments. (A) Supernatants of VEEV infected cells treated with various concentrations of BIOder (approximately 0.1, 1.0 and 10 μM) may be analyzed by plaque assay to determine the amount of infectious virus released. (B) U87MG astrocytes may be pretreated for approximately 2 hours with BIOder (approximately 0.1, 1.0, μM), infected with VEEV TC-83 at MOI approximately 0.1, and post-treated. Seventy hours post infection, CPE may be measured by MTT assay. Mock infected cells may be displayed at approximately 100% viability. (C) U87MG astrocytes may be treated as in panel A and cell viability assayed. Twenty-four hours post infection cell viability may be measured by MTT assay. Untreated (UT) cells may be displayed at 100% viability. (C)

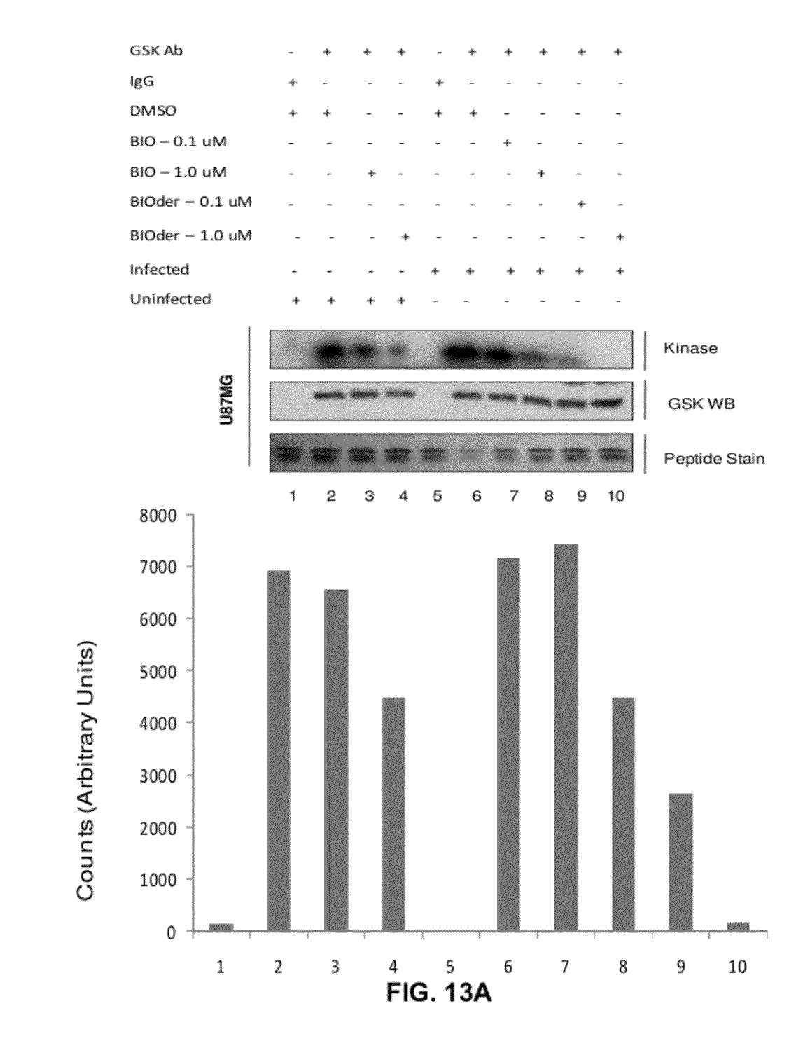

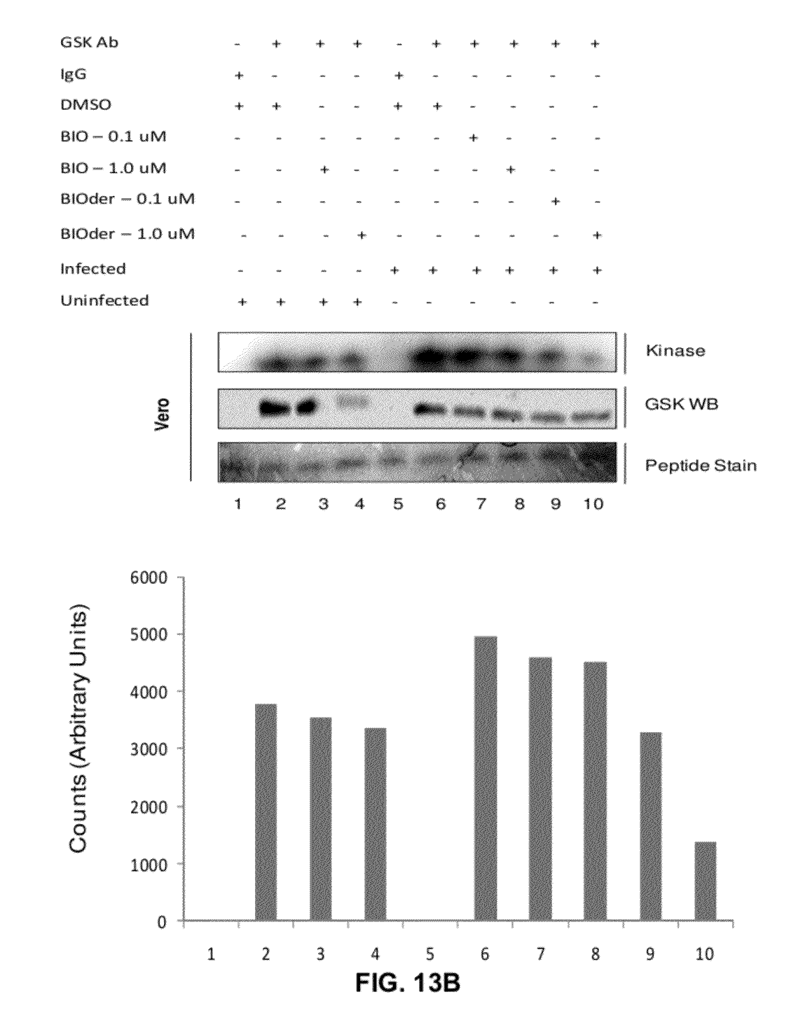

Examples FIG. 13A to FIG. 13B illustrate BIO and BIOder may inhibit GSK-3β in VEEV infected cells in accordance with some aspects of embodiments. (A) U87MG astrocytes and (B) Vero cells may be pretreated for approximately 2 hours with DMSO, BIO (approximately 0.1 and 1.0 uM), and/or BIOder (approximately 0.1 and 1.0 uM), infected with VEEV TC-83 at MOI approximately 0.1, and post-treated with DMSO, BIO, and/or BIOder. Mock infected cells may be treated with DMSO, approximately 1 uM BIO, and/or approximately 1 uM BIOder. Twenty-four hours post infection cells may be collected and protein extracts prepared. Approximately 1 mg of extract may be IPed at approximately 4° C. overnight with GSK-3β antibody. The next day, complexes may be precipitated with A/G beads for approximately two hours at approximately 4° C. IPs may be washed twice with TNE buffer and kinase buffer. Phosphorylation reactions may be performed with IP material and approximately 200 ng of glycogen synthase peptide 2 (Millipore) as substrate. Following incubation, samples may be separated by SDS-PAGE, dried and subjected to analysis using Molecular Dynamics Phosphor Imager software.

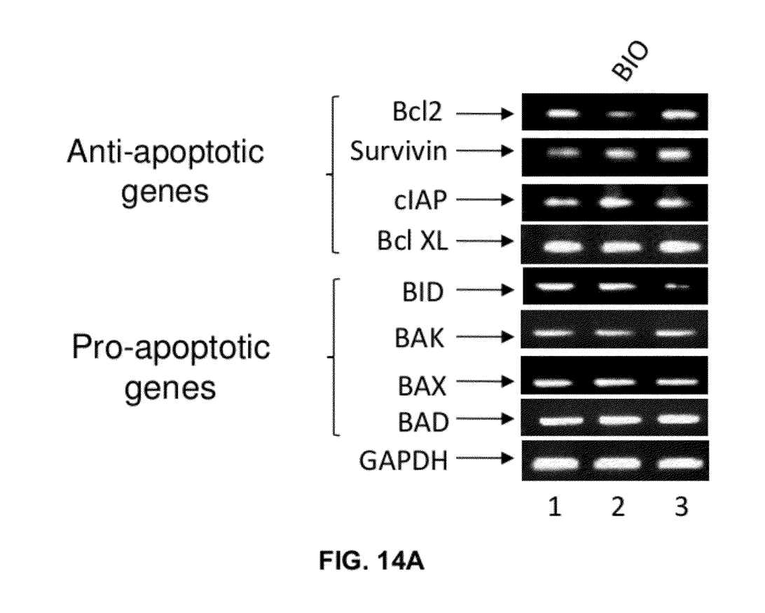

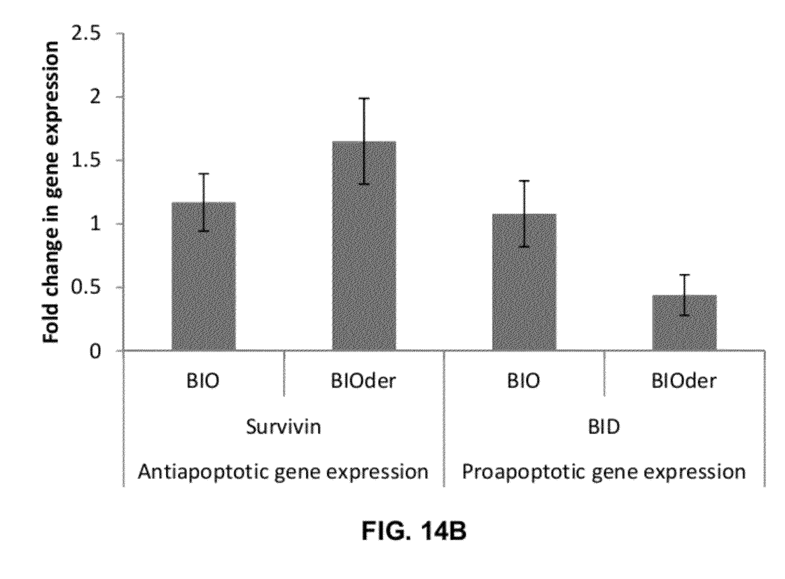

Examples FIG. 14A to FIG. 14B illustrate BIOder treatment may alter expression of apoptotic genes to promote survival of U87MG's. (A) U87MG's may be treated with approximately 1 μM BIO, approximately 1 μM BIOder, and/or DMSO and infected with VEEV TC-83. RNA may be harvested from infected cells approximately 24 hours post infection and analyzed by RT-PCR for expression of the indicated anti- and pro-apoptotic genes. (B) Band intensities corresponding to triplicate samples may be quantified and represented as fold change in gene expression over the DMSO control, with the DMSO control being set as a fold change of approximately 1.0.

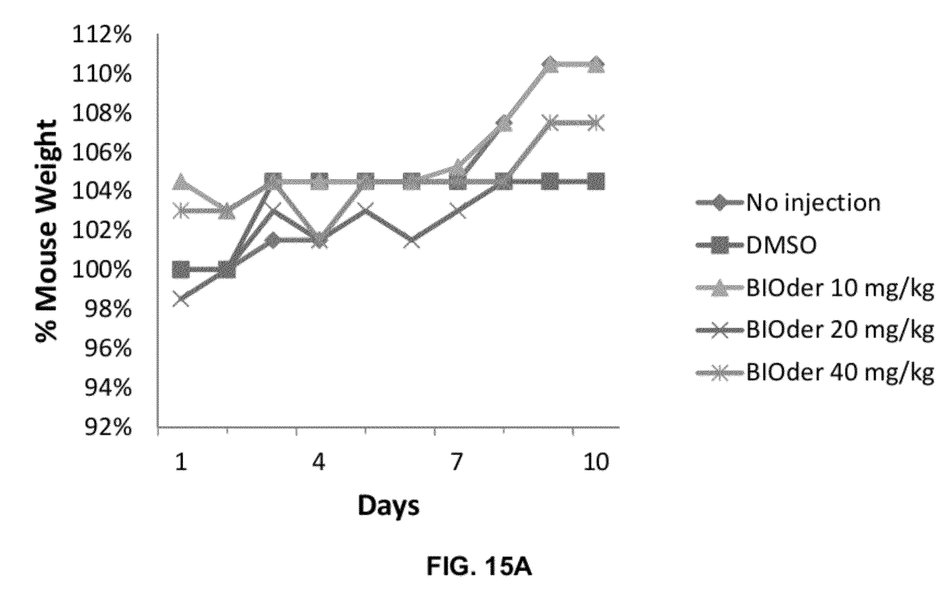

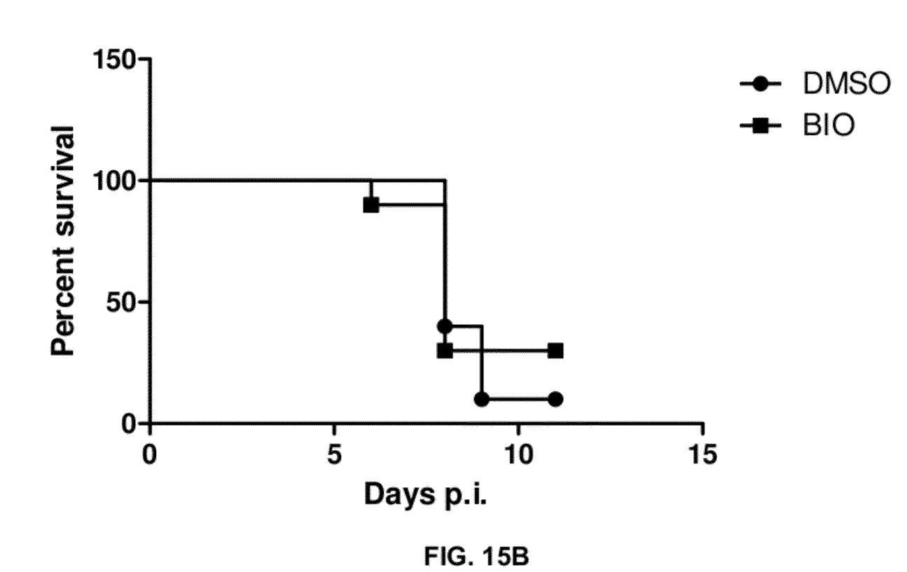

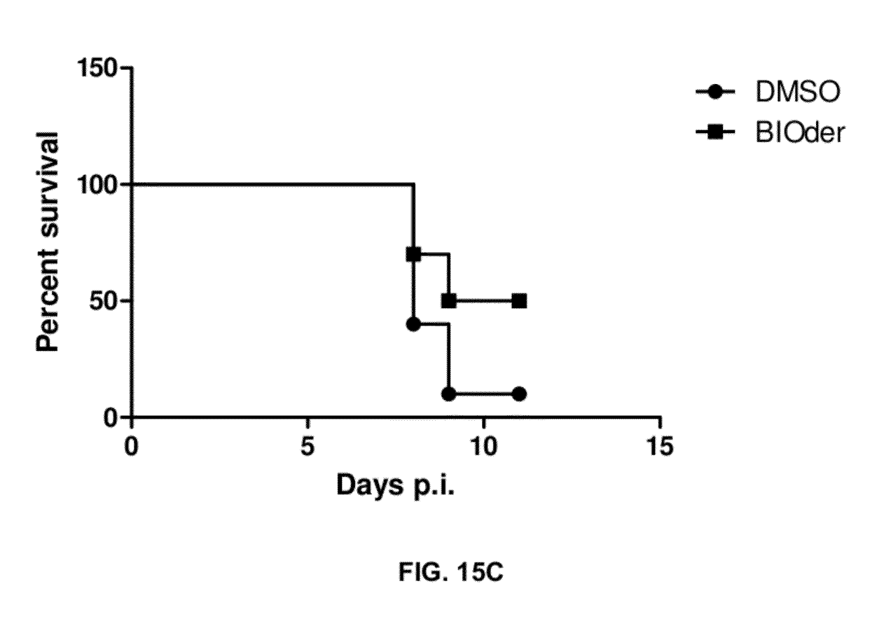

Examples FIG. 15A to FIG. 15B illustrate BIO and BIOder may inhibit VEEV cell death in vivo in accordance with some aspects of embodiments. (A) Female C3H/HeN mice may be treated subcutaneously with either DMSO or various concentration of BIOder (approximately 10 mg/kg, 20 mg/kg, 40 mg/kg) every day for approximately 5 days. Mice may be weighed daily and the average % of the mouse weight is plotted in panel A. (B) and (C) illustrate female C3H/HeN mice may be infected intranasally with approximately 5×LD50 (approximately 2×107 pfu) of VEEV TC-83. Groups of 10 mice may be treated SQ with vehicle, BIO (approximately 50 mg/kg) and/or BIOder (approximately 20 mg/kg) on days −1, 1, 3, and 5 and may be monitored for survival for approximately 14 days. Kaplan-Meier curves for survival between DMSO and BIO (panel B). Kaplan-Meier curves for survival between DMSO and BIOder (panel C). Significance may be determined using Mantel-Cox Log-rank test. P-value of 0.057 between control and BIOder.

### DESCRIPTION

Embodiments may relate to viral modulators and/or processes thereof. In some aspects of embodiments, a viral modulator may include a kinase modulator and/or process thereof. In one aspect of embodiments, a kinase modulator may include a glycogen synthase kinase (GSK) modulator, for example a GSK inhibitor. Embodiments may relate to a process of identifying a viral modulator, for example identifying a GSK inhibitor. Embodiments may relate to a process of making a viral modulator, for example making a GSK inhibitor. Embodiments may relate to a process of treating a subject with a viral modulator, for example employing GSK inhibitor dosing, dosing regimens and/or therapeutically effective amounts thereof. Embodiments may relate to contacting a cell and/or component thereof with a viral modulator, for example contacting a normal cell, an infected cell, a membrane of a cell, a compartment of a cell, a protein of a cell, a nucleotide of a cell, a metabolite of a cell and/or the like.

According to some aspects of embodiments, a viral modulator may include a GSK-3-β modulator and/or process thereof. In some aspects of embodiments, therapeutically effective amounts of a viral modulator, for example a GSK3-β inhibitor, may be employed. In another aspect of embodiments, a viral modulator may be employed as an HIV-1 transcriptional modulator, for example a GSK-3-β inhibitor employed as an HIV-1 transcriptional inhibitor.

Embodiments may relate to assaying and/or screening processes employing a viral modulator and/or process thereof, for example to identify and/or target a virus. In some aspects of embodiments, processes and/or compositions may be employed to assay and/or screen screen candidate compounds for kinase activity, such as GSK inhibitor activity, GSK3-β inhibitor activity, serine threonine kinase inhibitor activity and/or the like. In another aspect of embodiments, processes and/or compositions may be employed to assay and/or screen compounds for Tat-dependent LTR transcription activity, for example inhibitor activity. In further aspects of embodiments, processes and/or compositions may be employed to assay and/or screen candidate compounds for Tat-LTR transcriptional activity, for example inhibition activity.

According to some aspects of embodiments, a viral modulator, for example a GSK-3-β inhibitor, may be employed to modulate and/or treat neurodegenerative diseases in a subject. In one aspect of embodiments, a subject may include a mammal, primate, chordate, livestock, cell and/or components thereof. For example, a component may include a compartment (e.g., organ, organelle, cytoplasm, membrane, etc.), a system (e.g., CNS, PNS, circulatory system, respiratory system, etc.) and/or the like. In another aspect of embodiments, a viral modulator such as a GSK-3-β inhibitor may be employed to treat neurological disorders, for example HIV-associated dementia, mild neurocognitive disorder, asymptomatic neurocognitive impairment that are not prevented with HAART, VEEVassociated disorders, Parkinson's associated disorders, Alzheimer's associated disorders and/or the like.

According to some aspects of embodiments, a viral modulator, for example a GSK3-β inhibitor, may be employed in the treatment and/or suppression of inflammation. According to some aspects of embodiments, a GSK3-β inhibitor may be employed in the modulation, activation and/or suppression of transcription factors and/or co-factors including β-catenin, c-Jun, c-Myc, C/EBPα/β, NFATc, RelA and CREB.

According to some aspects of embodiments, a viral modulator may exhibit maximized potency. In one aspect of embodiments, a viral inhibitor may exhibit an IC₅₀ between approximately 0.02 nM and 0.06 nM. In another aspect of embodiments, a GSK3-β inhibitor may exhibit an IC₅₀ of approximately 0.03 nM.

Referring to example Table 1, a viral modulator, for example an inhibitor, activator and/or the like, may include 1: 2-{[[2-(5-bromo-2-oxo-1,2-dihydro-3H-indol-3-ylidene)hydrazino](oxo)acetyl]amino}benzoic acid, 2: N′˜1˜,N′˜4˜-bis(5-methyl-2-oxo-1,2-dihydro-3H-indol-3-ylidene)terephthalohydrazide, 3: 5-bromo-3-({2-[(2-oxo-1,2-dihydro-3H-indol-3-ylidene)amino]phenyl}imino)-1,3-dihydro-2H-indol-2-one, 4: 4-bromo-5-methyl-1H-indole-2,3-dione 3-oxime, 5: 4-bromo-5-methyl-1H-indole-2,3-dione 3-(N-phenylsemicarbazone), 6: 6-bromo-5-methyl-1H-indole-2,3-dione 3-[(6-bromo-5-methyl-2-oxo-1,2-dihydro-3H-indol-3-ylidene)hydrazone], 7: N′-(5-bromo-7-methyl-2-oxo-1,2-dihydro-3H-indol-3-ylidene)-2-chlorobenzohydrazide, 8: N′-(5-bromo-2-oxo-1,2-dihydro-3H-indol-3-ylidene)-2-chlorobenzohydrazide, 9: 5,7-dibromo-1H-indole-2,3-dione 3-(phenylhydrazone), 10: 5,7-dibromo-1H-indole-2,3-dione 3-oxime, 11: 2-chloro-N′-(5,7-dibromo-2-oxo-1,2-dihydro-3H-indol-3-ylidene)benzohydrazide, 12: 2-bromo-N′-(5,7-dibromo-2-oxo-1,2-dihydro-3H-indol-3-ylidene)benzohydrazide, 13: N′-(4-bromo-5-methyl-2-oxo-1,2-dihydro-3H-indol-3-ylidene)-3,5-dihydroxybenzohydrazide, 14: N′-(5-bromo-2-oxo-1,2-dihydro-3H-indol-3-ylidene)-2-methyl-3-furohydrazide, 15: N-(1-{[2-(5-fluoro-2-oxo-1,2-dihydro-3H-indol-3-ylidene)hydrazino]carbonyl}-2-phenylvinyl)benzamide, 16: N′-(5-bromo-2-oxo-1,2-dihydro-3H-indol-3-ylidene)-2,4-dichlorobenzohydrazide, 17: 4-bromo-5-methyl-1H-indole-2,3-dione 3-(phenylhydrazone), 18: 6-chloro-7-methyl-1H-indole-2,3-dione 3-oxime, 19: 4-chloro-7-methyl-1H-indole-2,3-dione 3-oxime, 20: 3-[(1H-indazol-5-ylamino)methylene]-1,3-dihydro-2H-indol-2-one, 21: 2-(5-bromo-2-methyl-1H-indol-3-yl)-N′-(2-oxo-1,2-dihydro-3H-indol-3-ylidene)acetohydrazide, 22: N′-(2-oxo-1,2-dihydro-3H-indol-3-ylidene)-3-phenyl-1H-pyrazole-5-carbohydrazide, 23: N′-(5-bromo-2-oxo-1,2-dihydro-3H-indol-3-ylidene)-3-phenyl-1Hpyrazole-5-carbohydrazide, 24: N′-(5,7-dibromo-2-oxo-1,2-dihydro-3H-indol-3-ylidene)-3-phenyl-1Hpyrazole-5-carbohydrazide, 25: N-[1-{[2-(5-bromo-2-oxo-1,2-dihydro-3H-indol-3-ylidene)hydrazino]carbonyl}-2-(3,4-dimethoxyphenyl)vinyl]benzamide, 26: N-[1-{[2-(5-bromo-7-methyl-2-oxo-1,2-dihydro-3H-indol-3-ylidene)hydrazino]carbonyl}-2-(2,5-dimethoxyphenyl)vinyl]benzamide, 27: 3-(4-methoxyphenyl)-N′-(2-oxo-1,2-dihydro-3H-indol-3-ylidene)-1Hpyrazole-5-carbohydrazide, 28: N′-(5-bromo-2-oxo-1,2-dihydro-3H-indol-3-ylidene)-3-(4-methoxyphenyl)-1H-pyrazole-5-carbohydrazide, 29: 3-(4-ethoxyphenyl)-4-methyl-N′-(2-oxo-1,2-dihydro-3H-indol-3-ylidene)-1H-pyrazole-5-carbohydrazide, 30: 3-(2-naphthyl)-N′-(2-oxo-1,2-dihydro-3H-indol-3-ylidene)-1H-pyrazole-5-Carbohydrazide, 31: N-(2-{[2-(5-bromo-2-oxo-1,2-dihydro-3H-indol-3-ylidene)hydrazino]carbonyl}phenyl)benzamide, 32: N′-(5-bromo-2-oxo-1,2-dihydro-3H-indol-3-ylidene)-3-methyl-1Hpyrazole-5-carbohydrazide, 33: 5-(5-bromo-2-oxo-1,2-dihydro-3H-indol-3-ylidene)-2,4-imidazolidinedione, 34: 5-bromo-5′-chloro-3,3′-biindole-2,2′(1H,1′H)-dione, 35: 5-chloro-3,3′-biindole-2,2′(1H,1′H)-dione, 36: 5-fluoro-3,3′-biindole-2,2′(1H,1′H)-dione, 37: 5-bromo-7-methyl-3,3′-biindole-2,2′(1H,1′H)-dione, and 38: 6-chloro-7-methyl-3,3′-biindole-2,2′(1H,1′H)-dione, a salt, isomer, tautomer, prodrug, composition and/or combinations thereof. In one aspect of embodiments, a composition may include one or more pharmaceutically acceptable carriers, for example an emulsion, paste, cream, lotion, gel, jelly, ointment, oil, aerosol, powder and/or solvent.

EXAMPLE TABLE 1
Example Compounds

| No | Structure | Approximate Properties | |
|---|---|---|---|
| 1 | 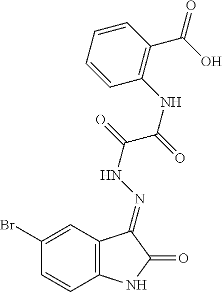 | Formula Molecular Weight LogP LogSW Rotatable Bonds Hdon Hacc tPSA | C₁₇H₁₁BrN₄O₅ 431 4.20 −5.93 4 4 6 137.0 |
| 2 | 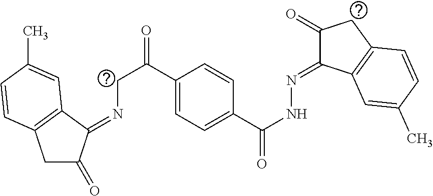 | Formula Molecular Weight LogP LogSW Rotatable Bonds Hdon Hacc tPSA | C₂₆H₂₀N₆O₄ 480 6.06 −7.88 4 4 6 141.1 |
| 3 | 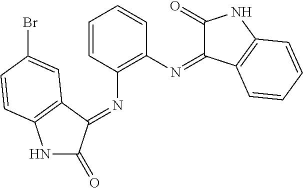 | Formula Molecular Weight LogP LogSW Rotatable Bonds Hdon Hacc tPSA | C₂₂H₁₃BrN₄O₂ 445 5.92 −7.50 2 2 4 82.9 |
| 4 | 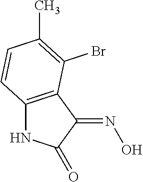 | Formula Molecular Weight LogP LogSW Rotatable Bonds Hdon Hacc tPSA | C₉H₇BrN₂O₂ 255 2.84 −3.49 0 1 3 61.7 |
| 5 | 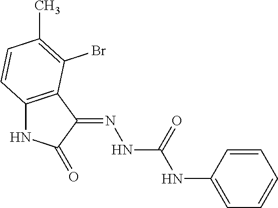 | Formula Molecular Weight LogP LogSW Rotatable Bonds Hdon Hacc tPSA | C₁₆H₁₃BrN₄O₂ 373 4.96 −6.16 2 3 3 82.6 |
| 6 | 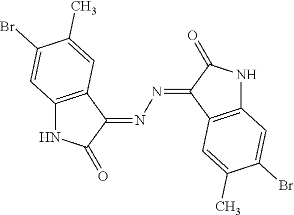 | Formula Molecular Weight LogP LogSW Rotatable Bonds Hdon Hacc tPSA | C₁₈H₁₂Br₂N₄O₂ 476 6.10 −7.88 1 2 4 82.9 |
| 7 | 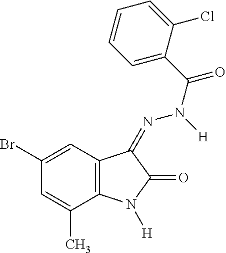 | Formula Molecular Weight LogP LogSW Rotatable Bonds Hdon Hacc tPSA | C₁₆H₁₁BrClN₃O₂ 393 5.67 −6.90 4 2 3 70.6 |
| 8 | 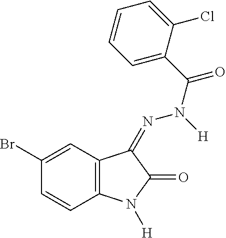 | Formula Molecular Weight LogP LogSW Rotatable Bonds Hdon Hacc tPSA | C₁₅H₉BrClN₃O₂ 379 5.17 −6.38 4 2 3 70.6 |
| 9 | 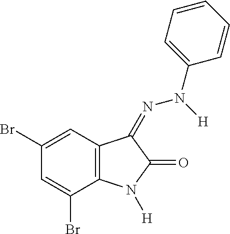 | Formula Molecular Weight LogP LogSW Rotatable Bonds Hdon Hacc tPSA | C₁₄H₉Br₂N₃O 395 5.32 −6.62 4 2 2 53.5 |
| 10 | 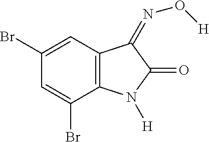 | Formula Molecular Weight LogP LogSW Rotatable Bonds Hdon Hacc tPSA | C₈H₄Br₂N₂O₂ [symbol] 320 [symbol] 3.26 [symbol] −4.32 [symbol] 1 2 [symbol] 3 61.7 [symbol] |
| 11 | 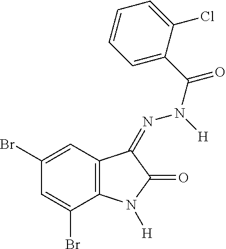 | Formula Molecular Weight LogP LogSW Rotatable Bonds Hdon Hacc tPSA | C₁₅H₈Br₂ClN₃O₂ 458 6.04 −7.69 4 2 3 70.6 |
| 12 | 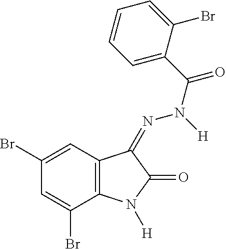 | Formula Molecular Weight LogP LogSW Rotatable Bonds Hdon Hacc tPSA | C₁₅H₈Br₃N₃O₂ 502 6.19 −8.14 4 2 3 70.6 |
| 13 | 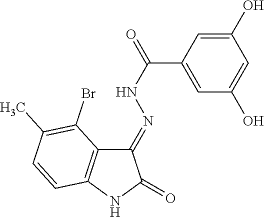 | Formula Molecular Weight LogP LogSW Rotatable Bonds Hdon Hacc tPSA | C₁₆H₁₂BrN₃O₄ 390 3.63 −4.57 2 4 5 111.0 |
| 14 | 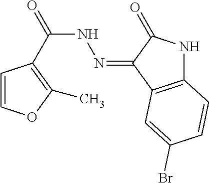 | Formula Molecular Weight LogP LogSW Rotatable Bonds Hdon Hacc tPSA | C₁₄H₁₀BrN₃O₃ 348 4.14 −5.27 2 2 4 83.7 |
| 15 | 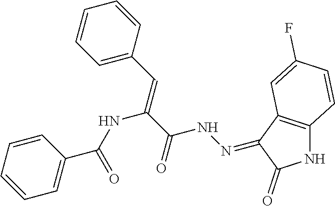 | Formula Molecular Weight LogP LogSW Rotatable Bonds Hdon Hacc tPSA | C₂₄H₁₇FN₄O₃ 428 4.52 −6.18 5 3 4 99.7 |
| 16 | 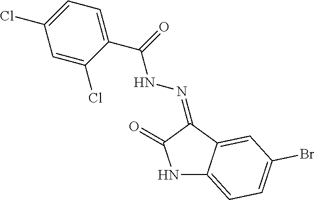 | Formula Molecular Weight LogP LogSW Rotatable Bonds Hdon Hacc tPSA | C₁₅H₈BrCl₂N₃O₂ [symbol] 413 [symbol] 5.89 [symbol] −7.24 [symbol] 2 [symbol] 2 [symbol] 3 [symbol] 70.6 [symbol] |
| 17 | 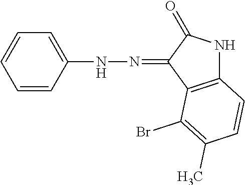 | Formula Molecular Weight LogP LogSW Rotatable Bonds Hdon Hacc tPSA | C₁₅H₁₂BrN₃O [symbol] 330 [symbol] 4.96 [symbol] −5.84 [symbol] 2 [symbol] 2 [symbol] 2 [symbol] 53.5 [symbol] |
| 18 | 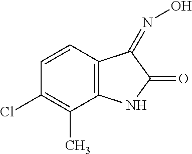 | Formula Molecular Weight LogP LogSW Rotatable Bonds Hdon Hacc tPSA | C₉H₇ClN₂O₂ 211 2.69 −3.04 0 1 3 61.7 |
| 19 | 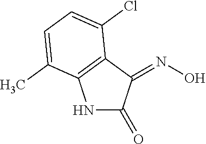 | Formula Molecular Weight LogP LogSW Rotatable Bonds Hdon Hacc tPSA | C₉H₇ClN₂O₂ 211 2.69 −3.04 0 1 3 61.7 |
| 20 | 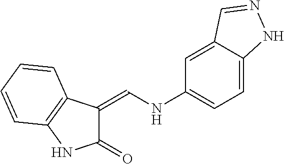 | Formula Molecular Weight LogP LogSW Rotatable Bonds Hdon Hacc tPSA | C₁₆H₁₂N₄O 276 3.33 −4.06 2 3 2 69.8 |
| 21 | 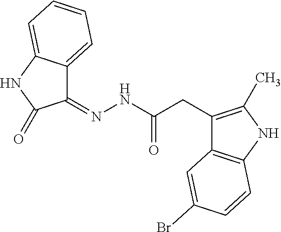 | Formula Molecular Weight LogP LogSW Rotatable Bonds Hdon Hacc tPSA | C₁₉H₁₅BrN₄O₂ 411 5.60 −6.98 3 3 3 86.4 |
| 22 | 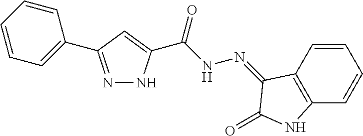 | Formula Molecular Weight LogP LogSW Rotatable Bonds Hdon Hacc tPSA | C₁₈H₁₃N₅O₂ 331 3.80 −4.86 3 3 4 99.2 |
| 23 | 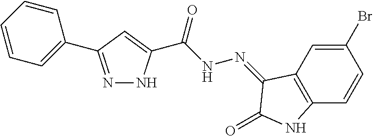 | Formula Molecular Weight LogP LogSW Rotatable Bonds Hdon Hacc tPSA | C₁₈H₁₂BrN₅O₂ 410 4.66 −6.17 3 3 4 99.2 |
| 24 | 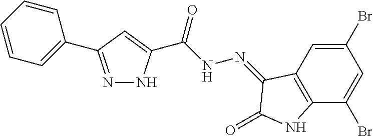 | Formula Molecular Weight LogP LogSW Rotatable Bonds Hdon Hacc tPSA | C₁₈H₁₁Br₂N₅O₂ 489 5.52 −7.48 3 3 4 99.2 |
| 25 | 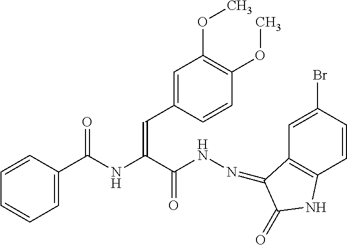 | Formula Molecular Weight LogP LogSW Rotatable Bonds Hdon Hacc tPSA | C₂₆H₂₁BrN₄O₅ 549 4.90 −7.39 5 3 6 118.1 |
| 26 | 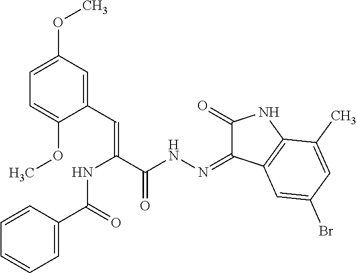 | Formula Molecular Weight LogP LogSW Rotatable Bonds Hdon Hacc tPSA | C₂₇H₂₃BrN₄O₅ 563 5.75 −8.22 5 3 6 118.1 |
| 27 | 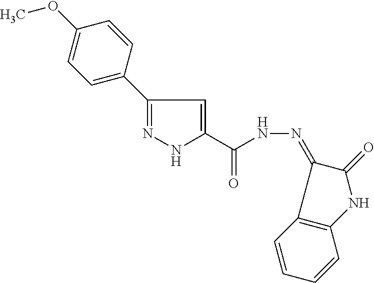 | Formula Molecular Weight LogP LogSW Rotatable Bonds Hdon Hacc tPSA | C₁₉H₁₅N₅O₃ 361 3.83 −5.11 3 3 5 108.5 |
| 28 | 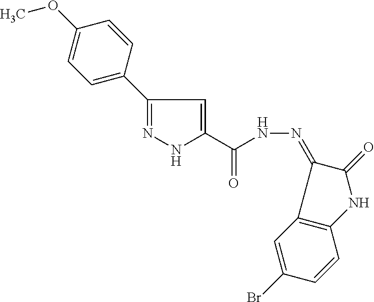 | Formula Molecular Weight LogP LogSW Rotatable Bonds Hdon Hacc tPSA | C₁₉H₁₄BrN₅O₃ 440 4.69 −6.41 3 3 5 108.5 |
| 29 | 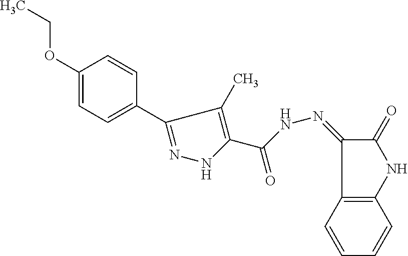 | Formula Molecular Weight LogP LogSW Rotatable Bonds Hdon Hacc tPSA | C₂₁H₁₉N₅O₃ 389 4.32 −5.73 3 3 5 108.5 |
| 30 | 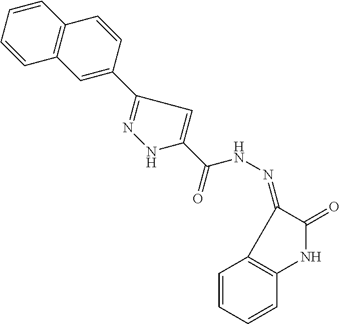 | Formula Molecular Weight LogP LogSW Rotatable Bonds Hdon Hacc tPSA | C₂₂H₁₅N₅O₂ 381 4.97 −6.22 3 3 4 99.2 |
| 31 | 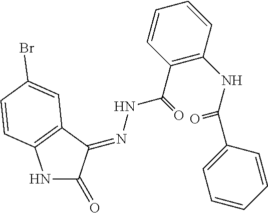 | Formula Molecular Weight LogP LogSW Rotatable Bonds Hdon Hacc tPSA | C₂₂H₁₅BrN₄O₃ 463 4.97 −6.82 4 3 4 99.7 |
| 32 | 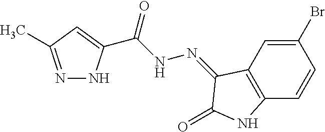 | Formula Molecular Weight LogP LogSW Rotatable Bonds Hdon Hacc tPSA | C₁₃H₁₀BrN₅O₂ 348 2.83 −4.16 2 3 4 99.2 |
| 33 | 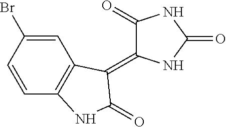 | Formula Molecular Weight LogP LogSW Rotatable Bonds Hdon Hacc tPSA | C₁₁H₆BrN₃O₃ 308 2.17 −3.30 0 3 3 87.3 |
| 34 | 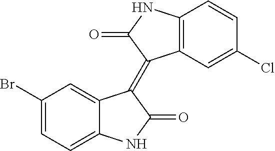 | Formula Molecular Weight LogP LogSW Rotatable Bonds Hdon Hacc tPSA | C₁₆H₈BrClN₂O₂ 376 4.13 −5.53 0 2 2 58.2 |
| 35 | 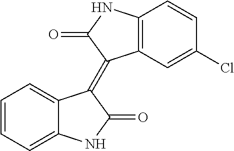 | Formula Molecular Weight LogP LogSW Rotatable Bonds Hdon Hacc tPSA | C₁₆H₉ClN₂O₂ 297 3.01 −4.19 0 2 2 58.2 |
| 36 | 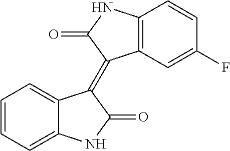 | Formula Molecular Weight LogP LogSW Rotatable Bonds Hdon Hacc tPSA | C₁₆H₉FN₂O₂ 280 2.44 −3.69 0 2 2 58.2 |
| 37 | 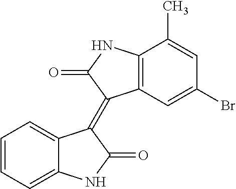 | Formula Molecular Weight LogP LogSW Rotatable Bonds Hdon Hacc tPSA | C₁₇H₁₁BrN₂O₂ 355 3.66 −5.29 0 2 2 58.2 |
| 38 | 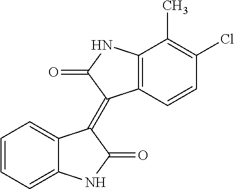 | Formula Molecular Weight LogP LogSW Rotatable Bonds Hdon Hacc tPSA | C₁₇H₁₁ClN₂O₂ 311 3.51 −4.76 0 2 2 58.2 |

[symbol]

indicates data missing or illegible when filed

### 1. Example Embodiment

### Modulation of HIV Replication and/or Toxicity

HIV-associated neurologic disease (HAND) may include microglial cell activation, astrocytosis, relatively decreased synaptic and/or dendritic density, and/or selective neuronal loss. In brains infected with HIV, neurons may die via an apoptotic mechanism. Although a substantial amount of neurodegenerative effects of HIV may be mediated through toxic cellular products released by infected microglia and/or dysfunctional astroglia, some viral proteins may themselves contribute directly to the neurotoxicity. Viral proteins such as Tat and/or gp120 may activate caspase-3 and/or endonuclease G (endo-G) in highly enriched cultures of striatal neurons in vitro. Both gp120 and Tat may be found in the brains of HIV patients.

The HIV regulatory protein Tat may be a mediator of HIV-induced neurotoxicity. Tat may be a transactivating, nonstructural viral nuclear regulatory protein including 101 amino acids encoded by two exons. Elongation of HIV-1 transcripts may be dependent on the association of HIV-1 Tat with the nascent RNA stemloop structure of the transactivating response element (TAR). This process may be accompanied by the binding of cellular proteins, including the P-TRFb complex of cyclin T1 or CDK9 to RNAP11 complex associated with HIV-1 LTR. Tat may be released by infected lymphoid and/or glial cells. Two forms of Tat may be released (e.g., Tat formed by the first exon only and Tat formed by both first and second exons). Tat may be cytotoxic to neurons.

The effect of Tat and/or gp120 on neurons may involve excitotoxic mechanisms. Targets for Tat may include αv integrin subunit-containing receptors, vascular endothelial growth factor-1 receptor (VEGF-1 receptor or flt-1), low-density lipoprotein receptor-related protein (LPR), and/or NMDA receptors (NMDA receptor activation may be secondary to GPCR activation). Interactions with excitatory amino acid receptors, with accompanying increases in Ca2+ and reactive oxygen species, may be detrimental. Tat injection into the brain, including the striatum, may cause gliosis and/or infiltration of macrophages, production of cytotoxic cytokines, and/or chemokines such as MCP-1. Intrastriatal Tat injections may induce neurodegenerative changes, which may precede peak increases in macrophages/microglia at approximately 24 h. Tat may be directly neurotoxic, as toxicity may occur in highly enriched cultures of striatal neurons. Relatively brief exposures to Tat may cause neuronal death. The core domain of Tat, amino acids 21-40, may induce cytopathic effects in monocytes and/or angiogenesis.

Glycogen synthase kinase (GSK)-3 may include a serine/threonine protein kinase, which may be a regulator of glycogen metabolism through the phosphorylation of glycogen synthase. GSK-3 may encode two isoforms, GSK-3α and GSK-3β, which may share approximately 97% sequence identity in their kinase domain but which may differ in their N- and C-terminus regions. GSK-3α/GSK-3β may be implicated in the regulation of glycogen synthesis, the Wnt signaling pathway, PI3K pathway, cell cycle control, transcriptional regulation and/or apoptosis. The ability of GSK-3α/GSK-3β to regulate this vast array of cellular processes may be related to its substrates, including glycogen synthase, Axin, β-Catenin, APC, cyclin D1, c-Jun, c-Myc, C/EBPα/β, NFATc, RelA, CREB and/or the like. GSK-3 may require phosphorylation of a serine residue at the C-terminal to the consensus site for some substrates such as glycogen synthase, but not for others such as β-Catenin. GSK-3β may be negatively regulated by PKB/AKT phosphorylation of Ser9. Modulating GSK-3β for the treatment of Alzheimer's disease and/or other neurological disorders may be of interest, for example due to its proapoptotic effects in neuronal cells.

Tat may induce GSK-3β activity. For example, up-regulation of GSK-3-β following the exposure of neurons to HIV-tat and down-stream mediator platelet-activating factor (PAF) may result in apoptosis. GSK-3β inhibitors such as lithium, SB 216763, and/or SB 415286 may protect cerebellar granule neurons from apoptosis. For example, GSK-3β activity may be reversed by the addition of a GSK-3β inhibitor such as lithium. Furthermore, the GSK-3β inhibitor lithium and valproic acid (VPA) may protect against Tat and/or gp120 mediated neurotoxicity. Rodent and/or human neurons exposed to culture fluids from HIV-1-infected monocyte-derived macrophages (MDMs) may be protected from cell death in the presence GSK-3β inhibitors (e.g., lithium, AR-A014418 and BIO). Lithium treatment may also result in neuronal protection and/or neurogenesis in SCID HIV-1 encephalitis (HIVE) mice. The role of GSK-3β in NF-kB regulated neuronal apoptosis may be leveraged, for example since neurons exposed to HIVADA-macrophage conditioned medium (MCM) may display relatively decreased NF-kB activity in a Tat dependent manner.

GSK-3β inhibition through lithium and/or Indirubin treatment may block NF-kB inhibition, the suppressive binding of RelA to HDAC3, and/or neuronal apoptosis. Lithium treatment may also inhibit HIV-1 replication of both T- and M-tropic viruses in PBMCs as well as TNF stimulated J1.1 cells. According to some aspects of embodiments, GSK-3β may be leveraged for the treatment of HAND as well as in the inhibition of HIV-1 replication in PBMCs. According to other aspects of embodiments, relatively small chemical molecules described herein may modulate GSK-3β and/or find use in the treatment of HAND.

Although GSK-3 inhibitors may be available, for example lithium, SB-216763, and SB-415286, lithium may be active in the 10-20 mM range and/or may inhibit other molecules including polyphosphate-1-phosphate, inositol monophosphatase, casein kinase-II, MAP kinase-activated protein kinase-2, and p38-regulated/activated kinase. SB-216763 and SB-415286 may be identified as GSK-3α inhibitors through a high throughput screen of the SmithKline Beecham compound bank against rabbit GSK-3α and/or inhibit human GSK-3 with IC₅₀'s of approximately 34 nM and 78 nM, respectively. In some aspects of embodiments, viral modulators may exhibit relatively lower potency compared to related strategies, for example exhibiting an IC₅₀ of approximately 5 nM.

According to some aspects of embodiments, a viral modulator, such as an inhibitor, may be employed to selectively target Tat neurotoxicity effects. In some aspects of embodiments, a viral modulator, including BIO and/or BIOder, may relatively inhibit Tat dependent transcription and/or Tat mediated neurotoxicity. In one aspect of embodiments, a viral modulator may be employed to minimize neurologic disease in a biological organism. In another aspect of embodiments a GSK-β inhibitor may be employed to minimize and/or treat HAND. In other aspects of embodiments, a viral modulator may maximize cell viability.

According to some aspects of embodiments, 6-bromoindirubin-3′-oxime (BIO) and/or 6-bromo-5-methyl-1H-indole-2,3-dione 3-[(6-bromo-5-methyl-2-oxo-1,2-dihydro-3H-indol-3-ylidene)hydrazone] (BIOder) may operate as a GSK3-β inhibitor. In one aspect of embodiments, BIO and/or BIOder may be employed to inhibit HIV-1 transcription as well as protect against Tat induced cell death. In further aspects of embodiments, BIO and/or BIOder may be employed as therapeutics for HAND. In other aspects of embodiments, BIO and/or BIOder may maximize cell viability.

According to some aspects of embodiments, a viral modulator, for example a GSK3-β inhibitor, may be administered alone and/or in combination with other viral modulators, for example with other GSK3-β inhibitors. In one aspect of embodiments, GSK3-β inhibitor BIO may be administered with GSK3-β inhibitor BIOder. In another aspect of embodiments, a viral modulator may be administered with one or more Tat-TAR interferers, for example with Novartis CGP 40336A, with multicyclic dyes including Hoechst 33258, DAPI and berenil, with Neomycin, and/or with any other desired compounds including WP631, Temacrazine and/or CDK inhibitors.

According to some aspects of embodiments, compounds may be screened. In one aspect of embodiments, BIO may be identified out of 3,280 compounds and determined as a GSK-3 inhibitor and/or Tat-dependent HIV-1 transcriptional inhibitor. In another aspect of embodiments, BIO's ability to inhibit HIV-1 transcription in an independent assay system including TZM-bl cells may demonstrate relatively enhanced potency, for example an IC₅₀ of approximately 40 nM. In further aspects of embodiments, BIO may include neuroprotective effects on Tat induced cell death, for example in rat mixed hippocampal cultures.

According to some aspects of embodiments, iterative screening and/or assays may be employed. In some aspects of embodiments, through further screening, one or more relatively potent viral modulators may be determined. In one aspect of embodiments, BIOder may exhibit relatively enhanced potency, for example an IC₅₀ of approximately 4 nM in primary macrophages and/or approximately 0.5 nM in U87MG cells when infected with HIV-1. In another aspect of embodiments, an MTT toxicity assay may demonstrate an inhibitory activity of more than approximately 10 uM in either primary cells or cell line. In further aspects of embodiments, an in vitro GSK-3β kinase inhibition assay may demonstrate that BIOder may exhibit a relatively low IC₅₀ of approximately 0.03 nM. In other aspects of embodiments, BIOder may include neuroprotective effects on Tat induced cell death, for example in rat mixed hippocampal cultures. Without being bound to any particular theory, viral modulators may demonstrate a dual mechanism of action with the ability to inhibit HIV-1 transcription as well as protect against Tat induced cell death, and/or may be employed as therapeutic for neurological disorders such as HAND.

According to some aspects of embodiments, any desired material and/or process may be employed. In some aspects of embodiments, any desired cell culture and/or reagent may be employed. In one aspect of embodiments, TZM-bl, U87MG, and/or 293T Cells may be grown and/or cultured to confluency in Dulbecco's modified Eagle's medium supplemented with approximately 10% heat-inactivated FBS, approximately 1% L-glutamine, and approximately 1% streptomycin/penicillin (Gibco/BRL, Gaithersburg, Md., USA). The latently infected promonocytic U1 cell line and the uninfected corresponding U937 cell line, as well as infected J1.1, ACH2 and their uninfected counterparts Jurkat and CEM (12D7) cells may be cultured up to approximately 1×10⁵ cells per ml (early log phase of growth) in RPMI-1640 medium supplemented with approximately 10% heat-inactivated FBS, approximately 1% L-glutamine, and approximately 1% streptomycin/penicillin. ACH2, J1-1 may contain a single integrated copy of HIV-1 genome, whereas U1 cells may harbor two copies (one wild type and one mutant) of the viral genome in parental U973 cells. All cells may incubated at approximately 37° C. and approximately 5% CO2.

According to some aspects of embodiments, the reporter T-cell lines JLTRG and JLTRG-R5 may be maintained at an average cell density of approximately 0.5×10⁶ cells/mL in RPMI 1640 (Mediatech, Herndon, Va., USA), supplemented with approximately 2 mM 1-glutamine, approximately 100 U/mL penicillin, approximately 100 μg/mL streptomycin, and approximately 10% heat-inactivated fetal bovine serum (FBS; HyClone, Logan, Utah, USA). TiGR cells may be derived by retroviral transduction of JLTRG cells with a retroviral MSCV-LTR driven Tat-expression vector.

According to some aspects of embodiments, the cell lines may be identical with respect to CD4 and CXCR4 expression, and/or JLTRG-R5 cells may express CCR5. CCR5 expression on JLTRG-R5 cells may be relatively stable in long-term culture. Over a two-year culture period, only approximately 30% of the cell may loose CCR5 expression. Complete CCR5 expression on a population basis may be easily reestablished by enriching CCR5-positive cells using anti-CCR5 antibody-coated magnetic beads (Dynal Biotech, Lake Success, N.Y., USA) or by fluorescence-activated cell sorting techniques. Prior to the infection experiments, the cells may be split to approximately 1×10⁵ cells/mL and then grown to a density of approximately 5×10⁵ cells/mL to maximize susceptibility to HIV-1 infection. JC53BL-13 cells (TZM-BL) may be cultured and infected as previously described. Briefly, cells may be maintained in Dulbecco's modified Eagle's medium (DMEM; Mediatech) supplemented with approximately 2 mM 1-glutamine, approximately 100 U/mL penicillin, approximately 100 μg/mL streptomycin, and approximately 10% heat-inactivated FBS.

According to some aspects of embodiments, PBMCs used to generate infectious viral supernatants may be isolated from the blood of healthy donors by Ficoll-Paque™ density gradient centrifugation (Amersham Biosciences, Uppsala, Sweden) and may be cultured in RPMI 1640 supplemented with approximately 10% heat-inactivated FBS, approximately 2 mM 1-glutamine, approximately 100 U/mL penicillin, and approximately 100 μg/mL streptomycin. PBMCs may initially be PHA/interleukin-2 stimulated and infected with HIV-1 89.6 strain approximately 4 days following stimulation. Antibodies may be purchased from BD Pharmingen (San Diego, Calif., USA). PHA-L may be obtained from Sigma (St. Louis, Mo., USA), and IL-2 may be purchased from Biosource International (Camarillo, Calif., USA).

According to some aspects of embodiments, any desired assay may be employed. In some aspects of embodiments, an n-well plate-based assay may be employed. In embodiments, a 384-well plate-based fluorometry assay may be employed. In one aspect of embodiments, all plate-based experiments may be performed in 384-well optical bottom black wall plates (Nalgen Nunc International, Rochester, N.Y., USA) and may be designed to obtain a final cell density of approximately 1×10⁶ cells/mL in a final volume of approximately 90 μL phenol red-free RPMI 1640 per well. This number may be obtained by titrating JLTRG-R5 cells over a range of cell numbers per well (between approximately 1×10³ and 1×10⁶ cell/well) and infecting the cells with HIV-1 NL4-3, followed by plate-based fluorometry approximately 3-6 days postinfection. The phenol red-free RPMI 1640 used in all experiments may be supplemented with approximately 2 mM 1-glutamine, approximately 100 U/mL penicillin, approximately 100 μg/mL streptomycin, and approximately 2% heat-inactivated FBS.

According to some aspects of embodiments, infections may be performed in the absence or presence of approximately 4 μg/mL diethylaminoethyl (DEAE)-dextran (molecular weight: approximately 5000), which on average may result in an approximate 20% increase in the obtained signal. The absence or presence of DEAE-dextran may not alter the quantitative ability of patient sera, TAK-779, or T-20 to neutralize HIV-1 infection. Analysis may be performed using a Synergy™ HT Multi-Detection Microplate Reader (Bio-Tek Instruments, Winooski, Vt., USA), equipped with the following filter set: excitation, approximately 488/20 nm; emission, approximately 525/20 nm.

According to some aspects of embodiments, to determine the Z′-factor, JLTRG-R5 or TiGR cells may be adjusted to a cell density of approximately 2×10⁶ cells/mL in phenol red-free RPMI 1640 supplemented with approximately 2% FBS, of which approximately 50 μL were loaded per well. The addition of approximately 50 μL of infectious viral cell culture supernatants per well [equal to a multiplicity of infection (MOI) of approximately 0.1] or approximately 50 μL of RPMI supplemented with approximately 2% FBS resulted in a final cell density of approximately 100,000 cells/well in a total volume of approximately 100 μL (approximately 1×10⁶ cells/mL).

According to some aspects of embodiments, a viral modulator, for example an inhibitor and/or an activator, may include 1: 2-{[[2-(5-bromo-2-oxo-1,2-dihydro-3H-indol-3-ylidene)hydrazino] (oxo)acetyl]amino}benzoic acid, 2: N′˜1˜,N′˜4˜-bis(5-methyl-2-oxo-1,2-dihydro-3H-indol-3-ylidene) terephthalohydrazide, 3: 5-bromo-3-({2-[(2-oxo-1,2-dihydro-3H-indol-3-ylidene)amino]phenyl}imino)-1,3-dihydro-2H-indol-2-one, 4: 4-bromo-5-methyl-1H-indole-2,3-dione 3-oxime, 5: 4-bromo-5-methyl-1H-indole-2,3-dione 3-(N-phenylsemicarbazone), 6: 6-bromo-5-methyl-1H-indole-2,3-dione 3-[(6-bromo-5-methyl-2-oxo-1,2-dihydro-3H-indol-3-ylidene)hydrazone], 7: N′-(5-bromo-7-methyl-2-oxo-1,2-dihydro-3H-indol-3-ylidene)-2-chlorobenzohydrazide, 8: N′-(5-bromo-2-oxo-1,2-dihydro-3H-indol-3-ylidene)-2-chlorobenzohydrazide, 9: 5,7-dibromo-1H-indole-2,3-dione 3-(phenylhydrazone), 10: 5,7-dibromo-1H-indole-2,3-dione 3-oxime, 11: 2-chloro-N′-(5,7-dibromo-2-oxo-1,2-dihydro-3H-indol-3-ylidene)benzohydrazide, 12: 2-bromo-N′-(5,7-dibromo-2-oxo-1,2-dihydro-3H-indol-3-ylidene)benzohydrazide, 13: N′-(4-bromo-5-methyl-2-oxo-1,2-dihydro-3H-indol-3-ylidene)-3,5-dihydroxybenzohydrazide, 14: N′-(5-bromo-2-oxo-1,2-dihydro-3H-indol-3-ylidene)-2-methyl-3-furohydrazide, 15: N-(1-{[2-(5-fluoro-2-oxo-1,2-dihydro-3H-indol-3-ylidene)hydrazino]carbonyl}-2-phenylvinyl)benzamide, 16: N′-(5-bromo-2-oxo-1,2-dihydro-3H-indol-3-ylidene)-2,4-dichlorobenzohydrazide, 17: 4-bromo-5-methyl-1H-indole-2,3-dione 3-(phenylhydrazone), 18: 6-chloro-7-methyl-1H-indole-2,3-dione 3-oxime, 19: 4-chloro-7-methyl-1H-indole-2,3-dione 3-oxime, 20: 3-[(1H-indazol-5-ylamino)methylene]-1,3-dihydro-2H-indol-2-one, 21: 2-(5-bromo-2-methyl-1H-indol-3-yl)-N′-(2-oxo-1,2-dihydro-3H-indol-3-ylidene)acetohydrazide, 22: N′-(2-oxo-1,2-dihydro-3H-indol-3-ylidene)-3-phenyl-1H-pyrazole-5-carbohydrazide, 23: N′-(5-bromo-2-oxo-1,2-dihydro-3H-indol-3-ylidene)-3-phenyl-1Hpyrazole-5-carbohydrazide, 24: N′-(5,7-dibromo-2-oxo-1,2-dihydro-3H-indol-3-ylidene)-3-phenyl-1Hpyrazole-5-carbohydrazide, 25: N-[1-{[2-(5-bromo-2-oxo-1,2-dihydro-3H-indol-3-ylidene)hydrazino]carbonyl}-2-(3,4-dimethoxyphenyl)vinyl]benzamide, 26: N-[1-{[2-(5-bromo-7-methyl-2-oxo-1,2-dihydro-3H-indol-3-ylidene)hydrazino]carbonyl}-2-(2,5-dimethoxyphenyl)vinyl]benzamide, 27: 3-(4-methoxyphenyl)-N′-(2-oxo-1,2-dihydro-3H-indol-3-ylidene)-1Hpyrazole-5-carbohydrazide, 28: N′-(5-bromo-2-oxo-1,2-dihydro-3H-indol-3-ylidene)-3-(4-methoxyphenyl)-1H-pyrazole-5-carbohydrazide, 29: 3-(4-ethoxyphenyl)-4-methyl-N′-(2-oxo-1,2-dihydro-3H-indol-3-ylidene)-1H-pyrazole-5-carbohydrazide, 30: 3-(2-naphthyl)-N′-(2-oxo-1,2-dihydro-3H-indol-3-ylidene)-1H-pyrazole-5-Carbohydrazide, 31: N-(2-{[2-(5-bromo-2-oxo-1,2-dihydro-3H-indol-3-ylidene)hydrazino]carbonyl}phenyl)benzamide, 32: N′-(5-bromo-2-oxo-1,2-dihydro-3H-indol-3-ylidene)-3-methyl-1Hpyrazole-5-carbohydrazide, 33: 5-(5-bromo-2-oxo-1,2-dihydro-3H-indol-3-ylidene)-2,4-imidazolidinedione, 34: 5-bromo-5′-chloro-3,3′-biindole-2,2′(1H,1′H)-dione, 35: 5-chloro-3,3′-biindole-2,2′(1H,1′H)-dione, 36: 5-fluoro-3,3′-biindole-2,2′(1H,1′H)-dione, 37: 5-bromo-7-methyl-3,3′-biindole-2,2′(1H,1′H)-dione, and 38: 6-chloro-7-methyl-3,3′-biindole-2,2′(1H,1′H)-dione. All compounds may be obtained from ChemBridge Corporation, and/or prepared in approximately 10 mM stock solution dissolved in DMSO.

According to some aspects of embodiments, screening and/or luciferase assay may be employed. In one aspect of embodiments, TZM-bl cells may be transfected with pc-Tat (approximately 1 μg) or silencing RNA (approximately 100 nM) against GSK-3B (Cell Signaling Technology, Danvers, Mass., USA) or luciferase (Dharmacon, Lafayette, Colo., USA) using the AttracteneLipofectamine reagent (InvitrogenQiagen, Chatsworth, Calif., USA) according to the manufacturer's instructions. TZM-bl cells may contain an integrated copy of the firefly luciferase gene under the control of the HIV-1 promoter (obtained through the NIH AIDS Research and Reference Reagent Program). The next day, cells may be treated with DMSO or the indicated compound at approximately 1 μM. Forty-eight hours post drug treatment, luciferase activity of the firefly luciferase may be measured with the BrightGlo Luciferase Assay (Promega, Madison, Wis., USA) and luminescence may be read from a 96 well plate on an EG&G Berthold luminometer (Berthold Technologies, Oak Ridge, Tenn., USA).

According to some aspects of embodiments, an MTT assay may be employed. In one aspect of embodiments, approximately fifty thousand cells may be plated per well in a 96-well plate and approximately the next day cells may be treated with approximately 1 μM compound or DMSO. Approximately forty-eight hours later, approximately 10 μA MTT reagent (approximately 5 mg/ml) may be added to each well and plates incubated at approximately 37° C. for approximately 2 hours. Next, approximately 100 μl of DMSO may be added to each well and the plate may be shaken for approximately 15 minutes at room temperature. The assay may be read at approximately 570 nM using a SpectraMax 340 plate reader (Molecular Devices, Sunnyvale, Calif., USA).

According to some aspects of embodiments, extracts may be prepared and/or blotting may be employed. In some aspects of embodiments, protein extracts may be prepared and/or immunoblotting may be employed. In one aspect of embodiments, whole cell extracts may be prepared. Cells may be collected and/or washed once with PBS and pelleted. Cells may be lysed in a buffer containing Tris-HCl approximately pH 7.5, approximately 120 mM NaCl, approximately 5 mM EDTA, approximately 0.5% NP-40, approximately 50 mM NaF, approximately 0.2 mM Na₃VO₄, approximately 1 mM DTT and one tablet complete protease inhibitor cocktail per approximate 50 ml. Lysis may be performed under ice-cold conditions, incubated on ice for approximately 30 minutes and spun at approximately 4° C. for approximately 5 minutes at approximately 14,000 rpm. The protein concentration for each preparation may be determined with a Bio-Rad protein assay kit (Bio-Rad Laboratories, Hercules, Calif., USA). Cell extracts may be resolved by SDS PAGE on an approximate 4-20% tris-glycine gel (Invitrogen, Carlsbad, Calif., USA).

According to some aspects of embodiments, proteins may be transferred to polyvinylidene difluoride microporous membranes using the iBlot dry blotting system as described by the manufacturer (Invitrogen). Membranes may be blocked with Dulbecco's phosphate-buffered saline (PBS) approximately 0.1% Tween-20+ approximately 3% BSA. Primary antibody against specified proteins may be incubated with the membrane in blocking solution overnight at approximately 4° C. Antibodies against GSK-3β (1V001) and β-actin (C-11) may be purchased from Santa Cruz Biotechnology (Santa Cruz, Calif., USA). Membranes may be washed twice with PBS+ approximately 0.1% Tween-20 and incubated with HRP-conjugated secondary antibody for approximately 1 hour in blocking solution. Presence of secondary antibody may be detected by SuperSignal West Dura Extended Duration Substrate (Pierce, Rockford, Ill., USA). Luminescence may be visualized on a Kodak 1D image station (Carestream Health, Rochester, N.Y., USA).

According to some aspects of embodiments, an RT assay may be employed. In one aspect of embodiments, viral supernatants (approximately 10 μl) may be incubated in a 96-well plate with RT reaction mixture containing approximately 1×RT buffer (approximately 50 mM Tris-HCl, approximately 1 mM DTT, approximately 5 mM MgCl₂, approximately 20 mM KCl), approximately 0.1% Triton, poly(A) (10-2 U), poly(dT) (10-2 U) and [³H]TTP. The mixture may be incubated overnight at approximately 37° C. and approximately 5 μl of the reaction mix may be spotted on a DEAE Filter mat paper (PerkinElmer, Shelton, Conn., USA) may be washed approximately four times with approximately 5% Na₂HPO₄ and three times with water, and then dried substantially completely. RT activity may be measured in a Betaplate counter (Wallac, Gaithersburg, Md.).

According to some aspects of embodiments, RT-PCR and/or primers may be employed. In one aspect of embodiments, for RNA analysis of CDK9/Cyclin T-dependent genes (MCL-1, IL-8, Cyclin D1) following drug treatments, total RNA may be isolated from U937 and U87MG cells employing Trizol (Invitrogen) according to the manufacturer's protocol. A total of approximately 1 μg of RNA from the RNA fraction may be treated with approximately 0.25 mg/ml DNase I for approximately 60 minutes, followed by heat inactivation at approximately 65° C. for approximately 15 minutes. A total of approximately 1 μg of total RNA may be employed to generate cDNA with the iScript cDNA Synthesis kit (Bio-Rad) using oligo-dT reverse primers.

According to some aspects of embodiments, an immunopercipitaiton and/or in vitro kinase assay may be employed. In one aspect of embodiments, for immunoprecipitation (IP), approximately 2 mg of extract from BIO or BIOder-treated (approximately 1, 10 μM) U937 cells may be immunoprecipitated at approximately 4° C. overnight with GSK-3β antibody. The next day, complexes may be precipitated with A/G beads (Calbiochem) for approximately two hours at approximately 4° C. IPs may be washed twice with appropriate TNE buffer and kinase buffer. Reaction mixtures (approximately 20 μA) may contain final concentrations: approximately 40 mM β-glycerophosphate at approximately pH 7.4, approximately 7.5 mM MgCl₂, approximately 7.5 mM EGTA, approximately 5% glycerol, [γ-32P]ATP (approximately 0.2 mM, approximately 1 μCi), approximately 50 mM NaF, approximately 1 mM orthovanadate, and approximately 0.1% (v/v) (3-mercaptoethanol.

According to some aspects of embodiments, phosphorylation reactions may be performed with IP material and approximately 200 ng of glycogen synthase peptide 2 (Millipore) as substrate in TTK kinase buffer containing approximately 50 mM HEPES (approximately pH 7.9), approximately 10 mM MgCl₂, approximately 6 mM EGTA, and approximately 2.5 mM dithiothreitol. Reactions may be incubated at approximately 37° C. for approximately 1 hour and stopped by the addition of approximately 1 volume of Laemmli sample buffer containing approximately 5% β-mercaptoethanol and ran on an approximate 4-20% SDS-PAGE. Gels may be subjected to autoradiography and quantitation employing a Molecular Dynamics PhosphorImager software (Amersham Biosciences, Piscataway, N.J., USA).

According to some aspects of embodiments, a transcriptional screening assay may be employed. In some aspects of embodiments, an HIV-1 transcriptional screening inhibitor assay may be employed. In one aspect of embodiments, an HTS compatible reporter cell line may be employed in which a previously developed, stably integrated chronically active HIV derived from a patient isolate drives eGFP expression controlled by a HIV-1 LTR(CUCY cells-Jurkat based) eGFP expression, thus serving as a direct and quantitative marker of HIV-1 expression. By including a second fluorescent protein which may be spectrally separated from GFP (DsRedExpress), and which may be controlled by an activation-independent promoter (MSCV-LTR), a reporter cell line may be generated in which simultaneous measurement of the influence of compounds on HIV-1 transcription (on-target) and general transcription (off-target) may be made. Such processes may minimize false positive hits, in which the compound would non-specifically inhibit global transcription.

According to some aspects of embodiments, a BSL2+/BSL3 facility may perform the actual drug screen, which may be relatively restrictive and/or substantially increase the cost of drug screening. In one aspect of embodiments, a prior attempt was made to establish a non-infectious drug screening system similar to CUCY cells, in which the integrated LTR-eGFP promoter construct in the parental JLTRG cells would be activated through stably integrated HIV-1 Tat expression plasmids. However, initial attempts to obtain high eGFP expressing cells through either stable transfection or retroviral transduction with Tat expression vectors may have failed. For example, retroviral vectors expressing HIV-1 Tat under the control of the murine leukemia promoter were found to efficiently transduce JLTRG cells, as indicated by the initially high measurable levels of eGFP expression, but then may have gradually lost eGFP expression, perhaps due to promoter methylation.

According to some aspects of embodiments, such issues may be addressed by employing retroviral vectors that may express HIV-1 Tat under the control of a murine stem cell virus promoter. In one aspect of embodiments, Tat may be cloned into a retroviral vecotor configured to be under the control of the murine stem cell virus promoter. Referring to example FIG. 2A, an iterative process may include retrovirally transducing JLTRG cells with the Tat vectors, sorting the transduced cells for high eGFP expression, and/or supertransducing this eGFP-positive population, etc., to obtain a cell population that generates a HIV-1 Tat driven eGFP fluorescence intensity similar to that observed in CUCY cells. Referring to example FIG. 2B, such cells may be transduced with retroviral vectors expressing relatively high levels of RFP, which similarly as in CUCY cells may serve as a simultaneously accessible marker for drug toxicities. Final single cell cloning for high eGFP/RFP expressing cells may result in the establishment of the clonal TiGR cell line. eGFP and DsRed expression in TiGR cells may be stable. Over approximately six months of continuous culture, an approximate 10% decrease of eGFP expression at the population basis may be observed. However, an approximately 100% double-positive cell population may be easily regenerated by cell sorting.

According to some aspects of embodiments, relatively good per-well cell density may be established. In one aspect of embodiments, titrating TiGR cells at various cell densities into 384 well plates may establish relatively good per-well cell density. The eGFP/RFP signals may increase in a linear manner from a cell density of approximately 5×10³ cells/well, with approximately 2×10⁵ cells (approximately 15-fold signal increase over background) may be relatively good for cell screening. Referring to example FIG. 2C, the Z′ may be approximately 0.89 at this cell density, indicating that the assay is relatively robust. The experimentally determined Z′ factor may be a dimensionless statistical value designed to reflect the dynamic range as well as the variation of the assay. The Z′ factor may be calculated according to equation (1):

Z′=1−(3σp+3σₙ)/|μₚ−μₙ) (1)

where 3σn may represent the standard deviation of the negative control samples, 3σp may represent the standard deviation of the positive samples, μn may be mean of the negative control samples and μp may be the mean of the positive samples. Z′=1 would be an ideal assay and 1>Z′>0.5 may be considered to be very good to excellent assays.

According to some aspects of embodiments, a TiGR-based HTS assay may be verified in a relatively small manually performed 1,000 compound screen, and/or may include known inhibitors of HIV-1 transcription (Ro24-7429, WP631) as controls, which may be efficiently detected (approximately 80-90% eGFP signal reduction on day 7). As TiGR cells may employ fluorescence intensities as quantitative markers for HIV-1 expression and drug toxicities, drug effects may be assessed intervention free. As such, the assay may not only determine cumulative inhibition at a defined time point, but also may determine the inhibitory/toxic onset kinetics of a respective compound, which may give additional insights into the possible drug efficiency. Therefore, this assay system may serve as a standardized platform to screen for Tat LTR transcriptional inhibitors.

According to some aspects of embodiments, TiGR cells may be employed, for example in an assay, screen and/or the like. In some aspects of embodiments, TiGR cells may be employed to identify HIV transcription modulators, for example Tat-dependent transcription inhibitors. In one aspect of embodiments, LOPAC Sigma-Aldrich (1280 compounds) and Spectrum-Microsource (2000 compounds) relatively small compound libraries may be screened employing the TiGR cells described above to identify HIV-1 transcription modulators, such as inhibitors. These libraries may be chosen as they may include pharmacologically active compounds, known drugs, experimental bioactives, and pure natural products which may provided a wide range of compounds with potential biological activity. Compounds may be diluted in DMSO and screened at approximately 10 uM for Tat transcriptional inhibition. TiGR cells may be analyzed at Days 0, 1, 2, 3, 4 and 7 post drug treatment to determine the GPF and RFP signals and those compounds that exhibited greater than approximately 30% inhibition of GFP expression without affecting the RFP expression were selected for further analysis.

According to some aspects of embodiments, the top candidates that may be identified through the TiGR cell system may be tested in TZM-bl cells, which contain an integrated LTR-luciferase reporter. Since TZM-bl system may be employed as a secondary screen, approximately 1 μM was selected as a cut-off. TZM-bl cells may be transfected with Tat followed by compound treatment the next day. Referring to example Table 2, results indicate % inhibition observed at approximately 1 uM as compared to the DMSO control.

EXAMPLE TABLE 2
Example Inhibitors

| | | % Inhibition |
|---|---|---|
| Inhibitor | Description and target | at 1 μM |
| Dactiniomycin | Antineoplastic, | 98 |
| | intercalating agent | |
| 6BIO | Glycogen synthase kinase | 62 |
| | 3α/β inhibitor | |
| Quinine ethyl carbonate | Antimalarial | 34 |
| Indirubin-3′-oxime | CDK inhibitor | 25 |
| Epirubicin hydrochloride | Antineoplastic | 17 |

The compound with the greatest relative inhibition may be the chemotherapeutic agent Dactinomycin, which may be a transcriptional inhibitor and anti-proliferation compound. Its mechanism of action may be non-specific, as it may bind to multiple DNA structures such as GC-rich duplexes, single-stranded forms, or hairpin forms, as well as interfering with RNA polymerase. Due to a relatively toxic nature, and therefore a possible limit as a HIV-1 therapeutic, we chose not to further pursue Dactinomycin, although such compound may nonetheless be employed as an inhibitor in some cases. Out of the remaining compounds, the GSK-3 inhibitor BIO (6-bromoindirubin-3′-oxime) may be ranked the second most relatively potent LTR transcriptional inhibitor. Therefore, these results indicate that BIO reproducibly may inhibit Tat dependent LTR transcription.

According to some aspects of embodiments, BIO may inhibit HIV-transcription without substantially inducing cellular activity. In some aspects of embodiments, BIO may be further tested using TZM-bl cells to determine its IC₅₀. TZM-bl cells may be transfected with Tat followed by treatment with various concentrations of BIO the next day. Referring to example FIG. 3A, luciferase assays may be performed approximately two days post-BIO treatment which may indicate that BIO may inhibit HIV-1 LTR Tat dependent transcription in a dose dependent manner, with an IC₅₀ of approximately 40 nM. MTT assays may be performed to determine the influence of BIO on cell viability. Referring to example FIG. 3B, BIO may not affect cell viability at the concentrations employed in our transcriptional assays. MTT assays may also be performed with BIO on multiple other cell lines, including uninfected and infected T-cells (CEM and ACH2), uninfected and infected monocytes (U937 and U1) and astrocytoma cells (U87MG). Minimal and/or no cellular toxicity may be observed upon treatment with BIO (approximately 1 uM), as compared to the DMSO control. Therefore, BIO may inhibit HIV transcription without inhibiting cellular viability.

According to some aspects of embodiments, activated PBMCs may be infected employing the dual tropic 89.6 virus and subsequently treated cells with vehicle (DMSO) or various concentrations of BIO (approximately 0.1, 0.5, and 1.0 μM) to determine whether BIO may have an effect on HIV-1 replication in primary cells. Treatment may be performed once. Cells may be maintained for approximately 14 days and supernatants may be collected at days 7 and 14 for RT analysis. Referring to example FIG. 3C, results may indicate that approximately 1.0 μM of BIO may inhibit virus replication by more than approximately 50% after 7 and 14 days. Cells may also be collected to determine the influence of BIO treatment on cell viability using PI staining/FACS analysis. Apoptosis may be determined through cell cycle analysis (sub G1 peak).

Referring to example FIG. 3D, uninfected PBMCs may display relatively low levels of apoptosis at both days 7 and 14. Infected PBMCs may also demonstrate low levels of apoptosis at day 7. However, at day 14 a relative increase in apoptosis may be observed, which without being bound to any particular theory, may be due to viral induced cell death as the DMSO control cells were also beginning to die. Collectively these results may indicate that the IC₅₀ of the BIO inhibition may be approximately 0.75 μM in HIV-1 infected PBMCs and that BIO may inhibit HIV replication without substantially inhibiting cellular viability.

According to some aspects of embodiments, Hit2Lead (Hit2Lead[dot]com) may be employed to provide BIO analogs. Referring to example Table 1 and FIG. 4A, thirty-eight commercially available BIO derivatives may be identified and/or tested at approximately 1 μM in the TZM-bl cells to determine their ability to modulate Tat-dependent LTR transcription. In some aspects of embodiments, analogs may increase viral transcription as compared to the DMSO control. In one aspect of embodiments, compounds 16 and 31 may exhibite superior agonist properties possibly, without being bound to any particular theory, due to removal of inhibitors from the promoter. In another aspect of embodiments, compounds that may exhibit agonist properties may include any compound exhibiting higher than approximately 5000 Luciferase units (e.g., DMSO level), for example compounds 3, 14, 16-17, 20, and/or 27-38. In further aspects of embodiments, compounds that may exhibit agonist properties may include any compound exhibiting higher than approximately 15000 Luciferase units, for example compounds 16 and 31.

According to some aspects of embodiments, analogs may display LTR transcriptional inhibition with different potencies. In one aspect of embodiments, compounds that may exhibit antagonist properties may include any compound exhibiting lower than approximately 5000 Luciferase units (e.g., DMSO level), for example compounds 4, 6, 15, 18, 21-22 and/or 25. In another aspect of embodiments, compounds that may exhibit antagonist properties may include any compound exhibiting lower than approximately 2500 Luciferase units, for example compounds 4, 6, and/or 18.

According to some aspects of embodiments, compound 6 (i.e., BIOder) identified in example Table 1, may exhibit particularly strong inhibition of LTR transcription. MTT assays may be performed employing compound 6 on multiple cell lines, including uninfected and infected T-cells (CEM and ACH2), uninfected and infected monocytes (U937 and U1) and astrocytoma cells (U87MG). Referring to example FIG. 4B, results may indicate that the transcriptional inhibition may not have been due to cellular toxicity, as relatively little change in viability was observed upon treatment with compound 6 as compared to the DMSO control. Compound 6 may be selected for follow-up inhibition analysis.

According to some aspects of embodiments, the effect of example compounds on the inhibition of HIV in monocytes and/or astrocytoma cells may be determined. In one aspect of embodiments, BIOder's effect in primary monocyte/macrophage infection and/or BIO may be tested. Referring to example FIG. 5A, the structure of BIO and BIOder are illustrated.

Referring to example FIG. 5B, monocyte/macrophages from a healthy donor, infected with HIV-1 dual tropic 89.6 for 7 days, may be employed. In some aspects of embodiments, both BIO and BIOder may be added to the media during the course of infection (once only). Lane 1 may show normal replication of HIV-1 as evidence by RT activity in supernatant and Lane 2 may be with DMSO control. Lane 3 employed BIO (approximately 10 nM) and 4-7 utilized BIOder at varying concentrations (approximately 0.1, 1, 10, 100 nM). There may be considerable inhibition with BIOder at approximately 10 nM. The IC₅₀ for BIOder in these cells may be approximately 4 nM. A similar assay may be employed in U87MG cells with BIOder at approximately 0.1, 1 and 10 nM. Again, inhibition may be mostly observed at approximately 1 nM with IC₅₀ at approximately 0.5 nM.

Referring to example FIG. 5C, MTT assays may be used employing monocyte/macrophage from two healthy donors and U87MG at approximately 10, 100, 1000 and 10,000 nM (e.g., lanes 2-5). There may be substantially no apparent toxicity in these cells. Collectively, these data may indicate that when searching for BIO analogs to inhibit HIV-1, 1 out of 38 compounds may be identified that may exhibit an IC₅₀ of between approximately 0.5 nM and 4 nM (e.g., which may depend on the cell type) and/or toxicity of more than approximately 10 μM. There may be an approximate 3 log difference between HIV inhibitory activity and possible cell toxicity.

According to some aspects of embodiments, BIOder may not inhibit cellular gene expression in the absence of Tat and/or may be specific to GSK-3β. In one aspect of embodiments, whether BIOder may be inhibitory to genes that require CDK9/Cyclin T may be determined. HIV-1 Tat may employ. CDK9/Cyclin T for its activation of transcription. We therefore determined if cellular gene expression may be sensitive to BIOder in treated cells. Referring to example FIG. 6A, RT/PCR results of 3 genes that require CDK9/Cyclin T for their transcription are illustrated. None of these genes may demonstrate a substantial decrease after BIOder treatment in U937 and/or U87MG cells in the absence of Tat. An increase in expression of MCL-1, IL-8, and Cyclin D1 may be observed in U937 cells following BIOder treatment.

According to some aspects of embodiments, we may determine if BIO and/or BIOder may inhibit GSK-3β kinase activity. U937 cells may be treated with BIO or BIOder, followed by immunoprecipitation with anti-GSK-3β. The Wed material may be employed in vitro kinase assays with glycogen synthase peptide 2 as the substrate. Referring to example FIG. 6B, results may indicate that BIOder and not BIO may be able to inhibit approximately 90% of the GSK-3β kinase activity at approximately 1 nM. The estimated in vitro IC₅₀ for BIOder in these kinase assays for GSK-3β may be approximately 0.03 nM. Collectively, this data may indicate that BIOder may be an effective GSK-3β inhibitor.

According to some aspects of embodiments, knockdown of GSK-3β may decrease viral transcription in cells. In some aspects of embodiments down-regulation of GSK-3β in cells may decrease viral gene expression and/or viral load in infected cells. In one aspect of embodiments, TZM-bl cells may be employed that may be transfected with siRNA against GSK-3β or luciferase in the presence or absence of Tat, and assayed for luciferase expression approximately 48 hours post-transfection. Referring to example FIG. 7A, results may indicate that siLuc or siGSK-3β may not control much of basal transcription in the Hela TZM-bl cells (e.g., lanes 1 and 2). However, siGSK-3β may substantially reduce Tat activated transcription in these cells (e.g., compare lanes 3 and 4). To confirm knockdown, whole cell extract of TZM-bl transfected with siRNAs may be run on an approximate 4-20% SDS-PAGE and Western blotted against GSK-3β and β-actin as control. Referring to example FIG. 7B, more than approximately 90% knockdown may be observed with siGSK-3β (e.g., lower insert, lane 4).

According to some aspects of embodiment, knockdown of GSK-3β may decrease virus release from HIV-1 infected cells. In some aspects of embodiments, J1-1 cells which may be Jurkat derived may be employed and/or may contain single copy integrated wild type virus, and release virus into the supernatant without addition of any external stimuli (e.g., TNF, or HDAC inhibitors). An experiment with either siLuc as control or siGSK-3β may be employed using electroporation. Referring to example FIG. 7C, results may demonstrate there may be a marked decrease of RT from cells treated with siGSK-3β at days 2 and 4. Collectively these data may indicate that knockdown of GSK-3β in either HeLa or Jurkat (J1-1) based cells may down-regulate HIV gene expression and viral production.

According to some aspects of embodiments, the effect of BIOder on the dox-dependent HIV-rtTA viruses (Tat/TAR specificity) may be determined. In some aspects of embodiments, the effect of BIOder may be specific to Tat function in HIV-1 expressing cells. In one aspect of embodiments, two sets of constructs may be obtained from the Berkhout lab which may have mutation in Tat/TAR sequence. These viruses may be induced with dox and full particles may be recovered in the supernatant. Briefly, the full-length, infectious HIV-1 molecular clone pLAI may be used for construction of an HIV-rtTA virus genome, the transcription of which may be controlled by dox. Referring to example FIG. 8A, the viral transcriptional elements TAR and Tat may be replaced by the prokaryotic tetO-rtTA elements.

According to some aspects of embodiments, TAR may be inactivated by mutation of multiple nucleotides in the single-stranded bulge and loop domains, the binding sites for Tat and cyclin T, respectively. Also, the inactive TAR motif may be inserted in both LTRs to minimize the chance of reversion to the wild-type virus by a recombination event. Inactivation of the Tat protein may be accomplished by introduction of the Tyr26Ala point mutation. This single amino acid change may result in a substantial loss of Tat transcriptional activity and virus replication. Thus, both LTRs may be modified, done in the wild-type (W) and mutant (Y) Tat backgrounds, which may result in four HIV-rtTA constructs: KWK, KYK, SWS, and SYS. KWK and KYK sets may be employed. The virus variant KWK may be most wild type-like since it may maintain the NF-B sites, SP1 sites, and a wild-type Tat protein, but it may have a mutation in TAR. The KYK clone may include similar promoter elements; however the Tat and TAR may be both mutated. These HIV-rtTA may replicate in a dox-dependent manner when transfected into either cell lines or primary PBMCs.

Referring to example FIG. 8B, experiments with the KWK and KYK clones may be performed in primary monocytes that may be differentiated into macrophages with PMA. Differentiated cells (approximately 3 days) may be electroporated with a approximately 20 μg of either KWK or KYK molecular clones and may be cultured without or with dox (approximately 1000 ng/ml). Virus production may be measured by RT on culture supernatant samples. Cells treated with dox may exhibit viral production from both KWK and KYK clones. When cells may be treated with BIOder, viral replication may be inhibited in the KWK (Tat+) and not KYK (Tat−) clone.

According to some aspects of embodiments, the effect of Tat and BIOder treatment on CDK9 responsive genes in primary macrophages may be determined. In one aspect of embodiments, MCL-1 may be employed, for example since there may be an observed increase in expression following BIOder treatment in the monocytic cell line U937. Referring to example FIG. 8C, MCL-1 expression may not substantially change following treatment with BIOder in the presence of KWK (Tat+); however a modest decrease (less than approximately 2-fold) in expression may be observed with BIOder treatment in the presence of KYK (Tat−) clone. These results further reinforce the notion that a functional Tat may be beneficial for the effect of 6BIOder in cells.

According to some aspects of embodiments, BIO may protect neural cultures from HIV-1 Tat protein. GSK-3 inhibitors such as lithium may have neuroprotective effects. In some aspects of embodiments, rat mixed hippocampal cultures may be preincubated with BIO prior to exposure to Tat. Cell death may be analyzed approximately 18 hours after Tat exposure by MTT assay. Referring to example FIG. 9A, Tat treatment may relatively reduce cell viability while BIO may be protective against Tat mediated neurodegeneration, with significant neuroprotective effects at approximately 1.0 and approximately 3.0 uM (p<0.05). However, there may be neurotoxicity observed at approximately 5.0 and approximately 10.0 uM of BIO, with a LD50 of approximately 4 uM.

According to some aspects of embodiments, BIOder may protect neural cultures from HIV-1 Tat protein. Referring to example FIG. 9B, BIOder may include a protective effect at approximately 1.0 and approximately 3.0 uM (p<0.05). Importantly, there was no neurotoxicity observed at higher concentrations of BIOder (approximately 5.0 and approximately 10.0 uM). These results indicate that BIO and BIOder may be able to protect neuronal cultures from Tat induced cell death. BIO and/or BIOder may be employed with one or more inhibitors that may have neuroprotective effects, for example with lithium.

### 2. Example Embodiment

### Modulation of VEEV Replication and/or Toxicity

Arthropod-borne viruses may be important causes of acute encephalitis and/or an may be an emerging worldwide problem with substantial risk for importation into new regions. Alphaviruses, including Venezuelan Equine Encephalitis Virus (VEEV), may cause disease in equine, humans and/or the like, which may exhibit overt encephalitis in a substantial percentage of cases. VEEV may be present in enzootic and/or epizootic strains, which may be different. Enzootic strains of VEEV may cycle between Culex mosquitoes and rodents. Horses may not serve as amplifying hosts for the enzootic VEEV and/or may become ill due to infection. However, horses may be relatively highly susceptible to epizootic VEEV (IA/B and IC subtypes), which may result in relatively high rates of mortality (20-80%). Horses may amplify the viruse, and resulting relatively high viremia may permit mosquito transmission, increasing equine disease and/or allowing the transmission to humans. For example, in 1995 VEEV re-emerged in Venezuela and Colombia causing an epidemic of 75-100,000 human cases. The increased circulation and/or spread of encephalitic arboviruses may demonstrate a need for understanding the pathogenesis of viral encephalomyelitis and/or identification of useful interventions.

The incubation period for VEEV may be approximately 2 days to 5 days. VEEV may be a cytoplasmically replicating virus that buds from the plasma membrane. VEEV may be an enveloped, non-segmented positive stranded RNA virus. The genome of VEEV may be approximately 11 kb in length and/or may encode two open reading frames (ORF). ORF1 may encode 4 nonstructural proteins (nsP1, nsP2, nsP3, and nsP4), which may play a role in viral replication and/or protein processing. nsP1 may be responsible for the capping and methylation of the viral plus-strand RNAs and/or for the regulation of minus strand RNA synthesis. nsP2 may be a viral protease responsible for cleavage of the P1234 polyprotein and/or may contain helicase activity. nsP3 may be a phospho-protein that may be impact minus strand RNA synthesis. nsP4 may be an RNA dependent RNA polymerase. ORF2 may encode 5 structural proteins; the capsid, the envelope glycoproteins (E1, E2, and E3), and the 6,000-molecular-weight (6K) protein. Many of the functional roles of viral proteins may have been studied in model alphaviruses, such as Sindbis and Ross River Viruses. For VEEV, there have been a number of studies on the capsid protein, demonstrating its ability to inhibit host transcription as well as nuclear import.

VEEV infection may result in CNS inflammation, including the induction of pro-inflammatory cytokines such as interleukin-1β (IL-1β, IL-6, IL-12, and/or tumor necrosis factor-α (TNF-α). The inflammatory response may contribute to neurodegeneration following encephalitic virus infection. Interestingly, many of the same cytokines may be influenced by glycogen synthase kinase-3β(GSK-3β) activity. GSK-3β activity may be important for the production of the pro-inflammatory cytokines, such as IL-6, IL-10, and TNF, and/or reduction of the anti-inflammatory cytokine IL-10. GSK-3 may include a serine/threonine protein kinase which may be important in energy homeostasis, insulin signaling, proliferation, apoptosis, neurobiology, development, and/or immunology. There may be interest in inhibiting GSK-3β for the treatment of Alzheimer's disease, and other neurological disorders, due to its ability to phosphorylate the microtubule associated Tau protein as well as influence inflammation. Moreover, GSK-3β inhibitors such as lithium, SB 216763, SB 415286, and/or BIOder may protect neurons from apoptosis. GSK-3β may also important for viral replication of some viruses, such as HIV and influenza. Knockdown of GSK-3β and/or inhibition through relatively small molecule compounds, such as BIO and/or BIOder, may inhibit HIV replication and Tat-dependent transcription.

According to some aspects of embodiments, relatively small molecule viral modulators, for example inhibitors, may inhibit both VEEV replication and VEEV induced cell death. In some aspects of embodiments, relatively small chemical molecules may be inhibitors of GSK-3β, which may include implication in inflammation and/or neurological disease. In one aspect of embodiments, BIOder may be employed as a VEEV therapeutic, for example as demonstrated by partial protection in mice from VEEV mortality.

According to some aspects of embodiments, viruses may be employed. VEEV TC-83 may be obtained from BEI resources. The TC-83 virus may be a live attenuated vaccine derivative of the Trinidad donkey (TRD) strain of VEEV, which may be derived by 83 serial passages of the virus in guinea pig heart cells. The genomes of TRD and/or TC-83 may differ at 12 nucleotide positions, and/or the attenuation of TC-83 may have been mapped to changes in the 5′-noncoding region and/or the E2 envelope glycoprotein. The replication of TC-83 may have been studied both in vitro and in vivo and may be a BSL-2 model for the fully virulent BSL-3 TRD VEEV

According to some aspects of embodiments, relatively small molecule viral modulators may be determined and/or employed. In some aspects of embodiments, viral modulators, for example an inhibitor and/or an activator, may include: 1: 2-{[[2-(5-bromo-2-oxo-1,2-dihydro-3H-indol-3-ylidene)hydrazino](oxo)acetyl]amino}benzoic acid, 2: N′˜1˜,N′˜-4-bis(5-methyl-2-oxo-1,2-dihydro-3H-indol-3-ylidene)terephthalohydrazide, 3: 5-bromo-3-({2-[(2-oxo-1,2-dihydro-3H-indol-3-ylidene)amino]phenyl}imino)-1,3-dihydro-2H-indol-2-one, 4: 4-bromo-5-methyl-1H-indole-2,3-dione 3-oxime, 5: 4-bromo-5-methyl-1H-indole-2,3-dione 3-(N-phenylsemicarbazone), 6: 6-bromo-5-methyl-1H-indole-2,3-dione 3-[(6-bromo-5-methyl-2-oxo-1,2-dihydro-3H-indol-3-ylidene)hydrazone], 7: N′-(5-bromo-7-methyl-2-oxo-1,2-dihydro-3H-indol-3-ylidene)-2-chlorobenzohydrazide, 8: N′-(5-bromo-2-oxo-1,2-dihydro-3H-indol-3-ylidene)-2-chlorobenzohydrazide, 9: 5,7-dibromo-1H-indole-2,3-dione 3-(phenylhydrazone), 10: 5,7-dibromo-1H-indole-2,3-dione 3-oxime, 11: 2-chloro-N′-(5,7-dibromo-2-oxo-1,2-dihydro-3H-indol-3-ylidene)benzohydrazide, 12: 2-bromo-N′-(5,7-dibromo-2-oxo-1,2-dihydro-3H-indol-3-ylidene)benzohydrazide, 13: N′-(4-bromo-5-methyl-2-oxo-1,2-dihydro-3H-indol-3-ylidene)-3,5-dihydroxybenzohydrazide, 14: N′-(5-bromo-2-oxo-1,2-dihydro-3H-indol-3-ylidene)-2-methyl-3-furohydrazide, 15: N-(1-{[2-(5-fluoro-2-oxo-1,2-dihydro-3H-indol-3-ylidene)hydrazino]carbonyl}-2-phenylvinyl)benzamide, 16: N′-(5-bromo-2-oxo-1,2-dihydro-3H-indol-3-ylidene)-2,4-dichlorobenzohydrazide, 17: 4-bromo-5-methyl-1H-indole-2,3-dione 3-(phenylhydrazone), 18: 6-chloro-7-methyl-1H-indole-2,3-dione 3-oxime, 19: 4-chloro-7-methyl-1H-indole-2,3-dione 3-oxime, 20: 3-[(1H-indazol-5-ylamino)methylene]-1,3-dihydro-2H-indol-2-one, 21: 2-(5-bromo-2-methyl-1H-indol-3-yl)-N′-(2-oxo-1,2-dihydro-3H-indol-3-ylidene)acetohydrazide, 22: N′-(2-oxo-1,2-dihydro-3H-indol-3-ylidene)-3-phenyl-1H-pyrazole-5-carbohydrazide, 23: N′-(5-bromo-2-oxo-1,2-dihydro-3H-indol-3-ylidene)-3-phenyl-1Hpyrazole-5-carbohydrazide, 24: N′-(5,7-dibromo-2-oxo-1,2-dihydro-3H-indol-3-ylidene)-3-phenyl-1Hpyrazole-5-carbohydrazide, 25: N-[1-{[2-(5-bromo-2-oxo-1,2-dihydro-3H-indol-3-ylidene)hydrazino]carbonyl}dimethoxyphenyl)vinyl]benzamide, 26: N-[1-{[2-(5-bromo-7-methyl-2-oxo-1,2-dihydro-3H-indol-3-ylidene)hydrazino]carbonyl}-2-(2,5-dimethoxyphenyl)vinyl]benzamide, 27: 3-(4-methoxyphenyl)-N′-(2-oxo-1,2-dihydro-3H-indol-3-ylidene)-1Hpyrazole-5-carbohydrazide, 28: N′-(5-bromo-2-oxo-1,2-dihydro-3H-indol-3-ylidene)-3-(4-methoxyphenyl)-1H-pyrazole-5-carbohydrazide, 29: 3-(4-ethoxyphenyl)-4-methyl-N′-(2-oxo-1,2-dihydro-3H-indol-3-ylidene)-1H-pyrazole-5-carbohydrazide, 30: 3-(2-naphthyl)-N′-(2-oxo-1,2-dihydro-3H-indol-3-ylidene)-1H-pyrazole-5-Carbohydrazide, 31: N-(2-{[2-(5-bromo-2-oxo-1,2-dihydro-3H-indol-3-ylidene)hydrazino]carbonyl}phenyl)benzamide, 32: N′-(5-bromo-2-oxo-1,2-dihydro-3H-indol-3-ylidene)-3-methyl-1Hpyrazole-5-carbohydrazide, 33: 5-(5-bromo-2-oxo-1,2-dihydro-3H-indol-3-ylidene)-2,4-imidazolidinedione, 34: 5-bromo-5′-chloro-3,3′-biindole-2,2′(1H,1′H)-dione, 35: 5-chloro-3,3′-biindole-2,2′(1H,1′H)-dione, 36: 5-fluoro-3,3′-biindole-2,2′(1H,1′H)-dione, 37: 5-bromo-7-methyl-3,3′-biindole-2,2′(1H,1′H)-dione, 38: 6-chloro-7-methyl-3,3′-biindole-2,2′(1H,1′H)-dione. All modulators may be prepared in 10 mM stock solution dissolved in DMSO.

According to some aspects of embodiments, relatively small molecule drug treatment may be employed. U87MG cells may be seeded at m cells per well, for example 10,000 cells per well, in an n-well plate, for example a 96-well plate, may be pretreated for approximately 2 hours with DMSO (final concentration of approximately 1%) and/or small molecule compounds in growth media. Cultured cells may be infected with VEEV TC83 at an MOI of approximately 0.1. Approximately one hour later, viral inoculums may be removed, cells washed multiple time (e.g., two times) with buffer such as PBS, and growth media may be supplemented with the relatively small molecule inhibitors added. Supernatants may be collected at a selected time, for example approximately 24 hours post-infection, and/or analyzed by plaque assays.

According to some aspects of embodiments, an assay may be employed. In some aspects of embodiments, a plaque assay may be employed. Vero cells may be plated in an n-well plate, for example 6 well plates, at a selected concentration, for example approximately 1×10⁶. Cells may be infected after a selected level of confluency. For example, when cells reach between approximately 90% and 100% confluency, cells may be infected as follows, for example in duplicates for each dilution. Viral supernatants may be diluted approximately 1:10 in complete DMEM media from approximately 10⁻¹ to 10⁻¹¹. Approximately four hundred μl of each viral dilution may be added to the cells. After the approximate one hour infection, an overlay of approximately 3 ml of an approximate 1:1 solution of approximately 0.5% agarose in diH2O with approximately 2×EMEM for plaque assays, containing approximately 5% FBS, approximately 1% L-Glutamine, approximately 2% penicillin/streptomycin, approximately 1% nonessential amino acids and approximately 1% sodium pyruvate may be added to each well, may be allowed to solidify and/or may be incubated at approximately 37° C., 5% CO2 and/or 48 hrs. After approximately 48 hours, cells may be fixed using approximately 4% formaldehyde for approximately 1 hr at room temperature. The agar plugs may be discarded and/or fixed cellular monolayers may be stained with approximately 1% crystal violet, 20% methanol solution for approximately 15 minutes, visualizing plaques. Averages may be taken from duplicates, with dilutions containing fewer than approximately 5 or more than approximately 100 plaques being discounted. The viral titer may be calculated as follows: pfu/ml=average of 2 plaque counts×2.5 (dilution factor)×dilution.

According to some aspects of embodiments, MTT assays may be employed. Approximately ten thousand U87MG cells may be plated per well, for example in a 96-well plate. Cells may be treated with approximately 1 μM compound and/or DMSO for example the following day. Approximately two hours later, cells may be infected with VEEV TC83 at an MOI of approximately 0.1 for approximately one hour. Following infection, medium containing compounds may be added back to the cells. MTT assay may be performed approximately 48 and/or 72 hours post-infection. For MTT assays, approximately 10 μl MTT reagent (approximately 50 mg/ml) may be added to each well, and/or plates may be incubated at approximately 37° C. for approximately 2 hours. Next, approximately 100 μl of DMSO may be added to each well, and/or the plate may be shaken for approximately 15 minutes at room temperature. The assay may be read at 590 nM, for example employing a DXT 880 plate reader (Beckman Coulter).

According to some aspects of embodiments, quantitative RT-PCR may be employed. U87MG cells may be infected with VEEV at an MOI of approximately 0.1. Approximately twenty-four hours later, supernatants may be collected for analysis of viral RNA. Viral RNA may be extracted using Ambion's MagMax viral RNA extraction kit and/or may be quantitated using q-RT-PCR with primers and probe for nucleotides 7931-8005 of VEEV TC-83. TC-83 RNA may be amplified (approximately 1 cycle 50° C. for approximately 30 minutes, approximately 1 cycle 95° C. for approximately 2 minutes approximately 40 cycles 95° C. for approximately 15 seconds and approximately 61° C. for approximately 60 seconds) using the ABI Prism 7000. Primer-pairs (forward TCTGACAAGACGTTCCCAATCA, reverse GAATAACTTCCCTCCGACCACA) and Taq-man probe (5′6-carboxyfluorescein-TGTTGGAAGGGAAGATAAACGGCTACGC-6-carboxy-N,N,N′,N′-tetramethylrhodamine-3′) may be known. Q-RT-PCR assays may be performed using Invitrogen's RNA UltraSense™ One-Step Quantitative RT-PCR System. The absolute quantification may be calculated based on the threshold cycle (Ct) relative to the standard curve.

According to some aspects of embodiments, immunoprecipitation and/or in vitro kinase assay may be employed. For immunoprecipitation (IP), approximately 2 mg of extract from BIO and/or BIOder-treated (approximately 0.1, 1.0 μM) U87MG and/or Vero cells may be immunoprecipitated at approximately 4° C. overnight with GSK-3β antibody. The next day, complexes may be precipitated with A/G beads (Calbiochem) for approximately two hours at approximately 4° C. IPs may be washed twice with appropriate TNE buffer and/or kinase buffer. Reaction mixtures (approximately 20 μl) may include the following final approximate concentrations: 40 mM β-glycerophosphate pH 7.4, 7.5 mM MgCl₂, 7.5 mM EGTA, 5% glycerol, [γ-32P]ATP (0.2 mM, 1 μCi), 50 mM NaF, 1 mM orthovanadate, and 0.1% (v/v) β-mercaptoethanol. Phosphorylation reactions may be performed with IP material and approximately 200 ng of glycogen synthase peptide 2 (Millipore) as substrate in TTK kinase buffer containing approximately 50 mM HEPES (approximate pH 7.9), approximately 10 mM MgCl₂, approximately 6 mM EGTA and/or approximately 2.5 mM dithiothreitol. Reactions may be incubated at approximately 37° C. for approximately 1 hour and stopped by the addition of approximately 1 volume of Laemmli sample buffer containing approximately 5% β-mercaptoethanol and analyzed by SDS-PAGE on an approximate 4-20% gel. Gels may be subjected to autoradiography and quantitation using Molecular Dynamics PhosphorImager software (Amersham Biosciences, Piscataway, N.J., USA).

According to some aspects of embodiments, RT-PCR may be analyzed. Total cellular RNA may be extracted employing Qiagen's RNeasy RNA extraction kit as per manufacturer's instructions. Approximately 1.0 ug of RNA may be employed to generate cDNA using iScript cDNA Synthesis kit (Bio-Rad) and oligo-dT reverse primers according to the manufacturer's instructions. Referring to example Table 3, resultant cDNA may be employed in a standard PCR reaction with primers against the pro- and anti-apoptotic genes indicated.

EXAMPLE TABLE 3
Example Primers

| Gene | Primer | Sequence |
|---|---|---|
| Bcl2 | Forward | AGGAAGTGAACATTTCGGTGAC |
| | Reverse | GCTCAGTTCCAGGACCAGG |
| Survivin | Forward | TTTCTCAAGGACCACCGCAT |
| | Reverse | CCAGCTCCTTGAAGCAGAAGAA |
| cIAP | Forward | TGGGAAGCTCAGTAACTGGGAA |
| | Reverse | GCATGTGTCTGCATGCTCAGAT |
| Bcl-XL | Forward | ATGGCAGCAGTAAAGCAAGC |
| | Reverse | CGGAAGAGTTCATTCACTACCTGT |
| BID | Forward | ACACTGTGAACCAGGAGTGAGT |
| | Reverse | AACAGCTTTGGAGGAAGCCA |
| BAK | Forward | TGGTCACCTTACCTCTGCAA |
| | Reverse | TCAAACAGGCTGGTGGCAAT |
| BAX | Forward | TGCTTCAGGGTTTCATCCAG |
| | Reverse | GGCGGCAATCATCCTCTG |
| BAD | Forward | AACCAGCAGCAGCCATCAT |
| | Reverse | CCACAAACTCGTCACTCATCCT |
| GAPDH | Forward | GGAAGGTGAAGGTCGGAGTCAA |
| | Reverse | CCTTGACGGTGCCATGGAAT |

PCR reactions may be carried as follows: approximately 94° C. for approximately 2 minutes, approximately 35 cycles of approximately 95° C. for approximately 30 seconds, approximately 54° C. for approximately 30 seconds, and approximately 72° C. for approximately 1 minute, followed by a final approximate 10 minute approximate 72° C. extension time. Amplified products may be separated in approximate 1% agarose gels stained with ethidium bromide and visualized using the Bio Rad Molecular Imager ChemiDoc XRS system (Bio-Rad). Band intensities may be calculated employing Quantity One 4.6.5 software (Bio Rad).

According to some aspects of embodiments, animal experiments may be performed. Approximately six to eight week old female C3H/HeN mice may be obtained from Charles River Laboratories, Wilmington, Mass. All experiments may be carried out in bio-safety level 2 (BSL-2) facilities and in accordance with the Guide for the Care and Use of Laboratory Animals (Committee on Care And Use of Laboratory Animals of The Institute of Laboratory Animal Resources, National Research Council, NIH Publication No. 86-23, revised 1996). The animal experiments may be performed under GMU IACUC protocol #0211. For toxicity experiments, female C3H/HeN mice may be treated subcutaneously with either DMSO or various concentration of BIOder (approximately 10 mg/kg, approximately 20 mg/kg, approximately 40 mg/kg) every day for approximately 5 days. Mice may be weighed daily and/or monitored for morbidity and mortality, including lethargy and ruffled fur. For infection experiments, female C3H/HeN mice may be infected intranasally with approximately 5×LD50 (approximately 2×10⁷ pfu) of VEEV TC-83. Groups of 10 mice may be treated subcutaneously with vehicle, BIO (approximately 50 mg/kg) and/or BIOder (approximately 20 mg/kg) on selected days, for example −1, 1, 3, and 5 and were monitored for survival for approximately 14 days. Significance may be determined employing Mantel-Cox Log-rank test.

According to some aspect of embodiments, BIO may inhibit VEEV replication. GSK-3β may be important for viral replication for viruses such as HIV and influenza. We determined whether GSK-3β inhibitors, such as BIO, may inhibit VEEV replication. U87MG cells may be pre-treated with BIO at various concentrations (approximately 0.1, 1.0 and 10□M), followed by infection with VEEV TC-83, and post-treatment with compounds. Viral supernatants may be collected approximately 24 hours post-infection and viral replication assayed by either q-RT-PCR, as illustrated in example FIG. 10A or plaque assays, as illustrated in example FIG. 10B. Results my demonstrate a dose dependent inhibition of VEEV replication.

According to some aspects of embodiments, the effect of BIO treatment on U87MG viability may be determined. Treatment of U87MG cells with BIO at approximately 1.0 μM may exhibit substantially no effect on cellular viability. Referring to example 12C, BIO treatment at approximately 0.1 μM may result in an increase in cellular viability, indicating that BIO treatment may increase cellular proliferation and/or inhibit cell death at relatively low concentrations. However, at approximately 10 μM of BIO, cellular viability may be decreased to an average of 56% viability, potentially limiting the therapeutic potential of BIO. These data may indicate that BIO may be a moderate inhibitor of VEEV replication.

According to some aspect of embodiments, BIO analogs which may inhibit VEEV replication and/or cytopathic effect may be determined. Due to the possible limited therapeutic potential of BIO, we determined relatively more potent BIO analogs. Hit2Lead (Hit2Lead[dot]com) may be employed to resolve 38 commercially available BIO analogs. The analogs may be tested employing q-RT-PCR to determine their ability to inhibit VEEV replication. U87MG cells may be pretreated with compounds (approximately □M), infected with VEEV TC-83 and post-treated with compounds. Viral supernatants may be collected approximately 24 hours post-infection and/or assayed for viral replication by q-RT-PCR.

According to some aspects of embodiments, compounds may exhibit superior antagonist properties. Referring to example FIG. 11A, compounds that may exhibit antagonist properties may include any compound causing lower than approximately 1×10⁶ VEEV genomic copies. In one aspect of embodiments, compounds that may exhibit antagonist properties may include any compound causing lower than approximately 1×10⁵ VEEV genomic copies, for example compounds 6, 8, 10, 16 and/or 19. In another aspect of embodiments, compounds that may exhibit antagonist properties may include any compound causing lower than approximately 1×10³ VEEV genomic copies, for example compounds 8 and/or 16. In embodiments, compound 6, 8, 10, 16 and/or 19 may demonstrate the relatively large inhibition of viral replication, decreasing viral replication by more than 10 fold.

According to some aspects of embodiments, inhibition observed may not be due to cellular toxicity. Cell viability assays may be performed on an analog. Referring to example FIG. 11B, results may indicate that most compounds may have relatively little to substantially no effect on cellular viability. However, compounds 16, 32, 33 and/or 34 may display a decrease in cellular viability, for example at 1 μM. Such compounds may be further studied and/or employed for targeted and/or direct cell death.

According to some aspects of embodiments, confirmation plaque assays may be employed. BIO analogs which may exhibit the relatively greatest inhibition of viral replication, without substantially inducing cellular toxicity, may be selected. Compounds 6, 8 and/or 19, for example, may exhibit approximately 10-fold inhibition of viral replication. Referring to example FIG. 11C, the ability of compounds 6, 8 and/or 19 may be confirmed to inhibit VEEV viral replication.

According to some aspects of embodiments, GSK-3β inhibitors may relatively increase proliferation and/or protect against cell death. This may be important for neurons, for example since they may be a non-replenishable cell population within the human body. In some aspects of embodiments, BIO analogs which may protect cells from the cytopathic effect (CPE) observed upon VEEV infection may be determined. All 38 BIO analogs illustrated in Table 1 may be assayed for the ability to protect cells from VEEV induced CPE.

Referring to example FIG. 11D, infected cells may display approximately 50% decrease in cell viability as compared to mock infected untreated cells. Many of the analogs may demonstrate minimized effect on CPE inhibition, and some derivatives may actually relatively increase observed CPE, such as analogs 5 and/or 24. A few analogs may inhibit CPE, for example compounds 6, 17, 28 and/or 31. Compound 6 may exhibit particularly strong inhibition of VEEV induced CPE as compared to all the other compounds. These results coupled with the viral replication inhibition data, may indicate that compound 6 may be an interesting VEEV therapeutic candidate based on its ability to inhibit viral replication and/or viral induced CPE.

According to some aspects of embodiments, BIOder may be characterized. Multiple concentrations of BIOder may be utilized to further characterize BIOder's therapeutic potential, and/or viral replication, viral induced CPE and/or cellular toxicity may be assayed. Referring to example FIG. 12A, BIOder may inhibit VEEV replication in a dose-dependent manner, and/or may exhibit an IC₅₀ of approximately 0.5 μM.

According to some aspects of embodiments, MTT assays may be performed to determine BIOder inhibition of viral induced cell death, for example to assess cell viability approximately 72 hours post-infection. Referring to example FIG. 12B, cells infected with VEEV and treated with DMSO may display a relative reduction in cellular viability by approximately 50%, while BIOder treated cells may exhibit a relative increase in cellular viability at substantially all concentrations employed. A relatively greater pronounced effect may be observed at concentrations between approximately 1.0 μM and 0.1 μM of BIOder. Mock infected cells may display substantially no inhibition of cellular viability.

Referring to example FIG. 12C, a relative increase in U87MG proliferation may be observed when cells are treated with substantial all concentrations of BIOder employed. U87MG cells may be treated with up to approximately 100 μM BIOder with no substantial effect on cellular viability. This is in agreement with the pro-proliferative activity of GSK-3β inhibitors. These results may indicate that the IC₅₀ for BIOder inhibition of VEEV induced CPE may be less than approximately 0.1 μM and/or that the CC₅₀ may be greater than approximately 100 μM, making BIOder a promising candidate. Collectively, these results may indicate that BIOder may be an inhibitor of VEEV induced CPE and/or VEEV replication.

According to some aspects of embodiments, BIO and/or BIOder may inhibit GSK-3β in VEEV infected cells. A series of kinase assays may be employed to determine the specificity and/or effectiveness of BIO and/or BIOder on GSK-3β from infected and uninfected cells. Referring to example FIG. 13A, U87MG cells, and to example FIG. 13B, Vero cells, may be infected with VEEV at MOI of approximately 0.1. Cells may be pre- and/or post-treated with compounds and collected approximately 24 hours post infection. Cells may be lysed and immunoprecipitated with control IgG and/or GSK-3β antibody. Immunoprecipitates may be bound to protein A and G agarose beads, washed and employed for in vitro kinase assays. A glycogen synthase peptide may be employed as a substrate.

According to some aspects of embodiments, substantially no kinase activity may be observed when uninfected or infected cells are immunoprecipitated with an IgG control antibody (e.g., lanes 1 and 5). GSK3-β antibody immunoprecipitations from DMSO treated cells may display a relatively robust phosphorylation of the glycogen synthase peptide (e.g., lanes 2 and 6). Cells treated with BIO may display a relatively slight decrease in kinase activity (e.g., lane 3 and 7). In contrast, treatment with BIOder may exhibit a much more relatively dramatic influence on the kinase activity of GSK3-β (e.g., lanes 4, 9, and 10). GSK3-β immunoprecipitated from infected cells may be relatively more susceptible to BIOder treatment (e.g., compare lanes 4 and 10). The effective inhibition may be observed in both U87MG and Vero cells, further indicating that BIOder may be the better therapeutic candidate.

According to some aspects of embodiments, BIOder treatment may alter expression of apoptotic genes to promote survival of U87MGs. BIOder may be relatively more effective than BIO in increasing viability of VEEV infected cells. Alterations in the expression patterns of pro- and/or anti-apoptotic genes following BIOder treatment may be attributed to the increased survival. RT-PCR analysis may be employed of DMSO, BIO and/or BIOder treated cells approximately 24 hours after VEEV infection. Cells may be pretreated with DMSO, BIO (approximately 1 μM) and/or BIOder (approximately 1 μM) for approximately two hours, after which they may be infected with VEEV (MOI: approximately 0.1). Cells may be continued to be treated with the inhibitors and DMSO for up to approximately 24 hours post infection at which point, the cells may be lysed and total RNA extracted.

According to some aspects of embodiments, RT-PCR may be carried out from DMSO and inhibitor treated cells with primers to anti-apoptotic genes (e.g., Bcl-2, Survivin, cIAP and Bcl-XL) and pro-apoptotic genes (e.g., BID, BAK, BAX and BAD). GAPDH may be measured as an internal control. Referring to example FIG. 14A, no significant changes in the expression of Bcl-1, cIAP and Bcl-XL may be observed, and/or a relative increase in the expression of survivin upon BIOder treatment was exhibited. Referring to example FIG. 14B, although treatment with BIO may relatively increase survivin expression, the fold increase in expression following BIOder treatment may be relatively higher over BIO. As illustrated in FIG. 14A, a relatively strong decrease in the expression of the pro-apoptotic gene BID following BIOder treatment may be observed. As illustrated in example FIG. 14B, measurement of relative expression levels between BIO and BIOder may reveal relatively stronger decrease in BID expression after BIOder treatment. BIOder may contribute to increased viability of VEEV infected cells by down regulating pro-apoptotic gene expression (BID) and/or up regulating anti-apoptotic gene expression (survivin).

According to some aspects of embodiments, BIO and/or BIOder may inhibit VEEV mortality in vivo, for example in mice. A toxicity study may be performed with BIOder, in vivo. Groups of 3 animals may be treated subcutaneously with DMSO, BIOder (approximately 10 mg/kg), BIOder (approximately 20 mg/kg) and/or BIOder (approximately 40 mg/kg) every day for approximately five days. Mice may be monitored for signs of toxicity including lethargy, ruffling of coats, and/or weight loss. Substantially no signs of toxicity may be observed in any of the treatment groups.

Referring to example FIG. 15A, the average % mouse weight may be illustrated, where substantially no treatment group showed weight loss and/or all groups gained weight over the 10 day period. Based on these data, the dosage of BIOder chosen for our infection study may be approximately 20 mg/kg as our in vitro data may demonstrate it to be a relatively potent inhibitor, where as low as approximately 0.1 μM of BIOder may inhibit VEEV induced CPE. A relatively higher dose of BIO (approximately 50 mg/kg) may be chosen due to relatively less potent inhibition of VEEV and BIO being previously utilized in vivo at 50 mg/kg.

According to some aspects of embodiments, a VEEV TC-83 mouse model may be employed to determine if BIO or BIOder may protect against VEEV the VEEV TC-83 mouse model was utilized. Groups of 10 mice may be treated subcutaneously with vehicle, BIO (approximately 50 mg/kg) and/or BIOder (approximately 20 mg/kg) on days −1, 1, 3, and 5, and monitored for approximately 14 days. Referring to example FIG. 15B and FIG. 15C, vehicle treated animals had a approximate 10% survival rate, BIO treatment resulted in a approximate 30% survival rate and BIOder treatment resulted in an approximate 50% survival rate. Mean time to death (MTD) between the control and BIOder treated animals increased from approximately 8 days to approximately 10 days, and had a survival differential of approximately 40%, being statistically significant (p-value of 0.057). In contrast, BIO MTD was approximately 8 days and a survival differential of approximately 20%, indicating no obvious significance (p-value of 0.733). Thus, BIO may not exhibit efficacy delivered subcutaneously against VEEV TC-83 when the treatments are given as described above. BIOder may exhibit more promise than BIO. Increased efficacy may be achieved with a more frequent treatment regimen (e.g., approximately once daily), higher dosage (e.g, approximately 40 mg/kg), or different route of compound administration (e.g., intraperitoneal). These data demonstrate that BIOder treatment may reduce VEEV induced mortality.

### 3. Further Example Embodiments

### Viral Modulators

According to some aspects of embodiments, a method may include contacting one or more biological systems with one or more viral modulators. In one aspect of embodiments, a biological system may include a cell (e.g., a neural cell, epithelial cell, muscle cell, etc.), a system (e.g., a nervous system), components thereof (e.g., protein, compartment, etc.) and/or the like. In another aspect of embodiments, a compartment of a biological system may include an organ, organelle, cytoplasm, membrane and/or the like. In further aspects of embodiments, a system of a biological system may include a CNS, PNS, circulatory, respiratory, lymphatic system and/or the like. In other aspects of embodiments, a biological system may include a normal cell, an infected cell and/or the like. In more aspects of embodiments, contacting may include contact with a biological system and/or components thereof, for example contact with a protein of a cell, a nucleotide of a cell, a metabolite of a cell and/or the like.

According to some aspects of embodiments, a biological system may be configured to be infected by one or more viruses. In one aspect of embodiments, a biological system may be configured to be infected by (e.g., HIV-1). In another aspect of embodiments, a biological system may be configured to be infected by VEEV. In further aspects of embodiments, a biological system may be configured to form one or more proteins, nucleic acids and/or metabolites, which may interact and/or may be modulated by one or more viral modulators.

According to some aspects of embodiments, a biological system may include a subject, for example a human subject. In one aspect of embodiments, a subject may include a healthy subject, an infected subject, a subject at risk for an infection and/or the like. In another aspect of embodiments, contacting may include administering a therapeutically effective amount of one or more viral inhibitor compounds (e.g., inhibitor) to a subject.

According to some aspects of embodiments, a biological system may include an in vitro system. In one aspect of embodiments, an in vitro system may include an assay system and/or a screen system. In another aspect of embodiments, for example, an assay system and/or a screen system may include one or more cells, compartments and/or components thereof. In further aspects of embodiments, an assay system and/or a screen system may be contacted with one or more viral modulators to target infection, screen for infection and/or determine modulation of infection.

According to some aspects of embodiments, a cell may be transduced with an expression vector. In one aspect of embodiments, for example for HIV, a JLTRG cell may be transduced with a Tat expression vector. In another aspect of embodiments, an expression vector may be under the control of a promoter, for example a Tat expression vector under the control of a murine stem cell promoter. In further aspects of embodiments, a selected cell may be isolated from transduced cells, for example isolating an eGFP-expressing cell from JLTRG transduced cells.

According to some aspects of embodiments, one or more other additional transduction on an isolated expressing cell may be performed employing an expression vector, for example on the isolated eGFP-expressing cell employing the Tat expression vector. In one aspect of embodiments, an isolated expressing cell may be further transduced with one or more other expression vectors, for example transducing an isolated eGFP-expressing cell employing an RFP-expressing vector. In another aspect of embodiments, single cell cloning may be performed for expressing cells to isolate assay and/or screen cells, for example single cell cloning may be performed for eGFP/RFP expressing cells to isolate a TiGR cell. In further aspects of embodiments, an isolated assay and/or screen cell may be contacted with one or more compounds to target infection, screen for infection and/or determine modulation of infection. In embodiments, for example, one or more TiGR cells may be contacted with one or more viral modulators to determine Tat modulation.

According to some aspects of embodiments, a viral modulator may include a compound represented by the structure illustrated in example FIG. 1A.

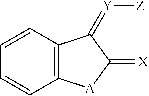

According to some aspects of embodiments, X, Y or A may include one or more alkyl, alkylene, alkene, aryl, heteroaryl, halogen, hydrogen, hydroxyl and/or the like, and combinations thereof. In one aspect of embodiments, X may be a Group 16 element, for example Oxygen (O). In another aspect of embodiments, Y and A may be a group 15 element, for example Nitrogen (N). In further aspects of embodiments, for example when A is Nitrogen, the Nitrogen atom may be bonded with any element and/or compound, for example with a Hydrogen, alkyl, alkylene, alkene, aryl, heteroaryl, halogen, hydroxyl and/or the like.

According to some aspects of embodiments, Y may be bonded with any desired element and/or compound Z. In some aspects of embodiments, Z may include any alkyl, alkylene, alkene, aryl, heteroaryl, halogen, hydrogen, hydroxyl and/or the like, and combinations thereof. In one aspect of embodiments, Z may include N—, NH—, NH—C(O), NH—C(O)-aryl, NH-aryl, OH, NH—C(O)—NH-aryl, N-heteroaryl, NH—C(O)-haloaryl, NH—C(O)-straight or branched chain hydrocarbon, NH-heteroaryl and/or combination thereof. In another aspect of embodiments, an aryl may include a functional group and/or substituent derived from an aromatic ring, for example a derivative of benzene. In further aspects of embodiments, a benzene derivative may include, for example, chlorobenzene, dibromobenzene and/or the like.

According to some aspects of embodiments, a heteroaryl may include an aromatic ring including carbon atoms, hydrogen atoms, independently selected heteroatoms, for example from Nitrogen, Oxygen, Sulfur and/or the like. In one aspect of embodiments, a heteroaryl may include a furan, indole, pyrazole, imidazole and/or the like. In another aspect of embodiments, substituents may be included at any position of a viral modulator compound, for example any position along a chained alkane, any ring position along an aryl and/or the like. In further aspect of embodiments, two or more substitutions may at adjacent, random, and/or non-adjacent positions in a viral modulator compound.

According to some aspects of embodiments, an HIV viral inhibitor compound may include a compound represented by the structure illustrated in example FIG. 2A.

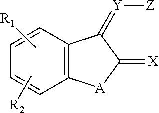

According to some aspects of embodiments, R₁ and/or R₂ may include one or more alkyl, alkylene, alkene, aryl, heteroaryl, halogen, hydrogen, hydroxyl and/or the like, and combinations thereof. In one aspect of embodiments, R₁ and R₂ may be on adjacent ring positions, for example positions 4 and 5, 5 and 6 and/or 6 and 7 of an inidole. In another aspect of embodiments, R₁ and/or R₂ may be a Group 17 element, for example Bromine, and the other of R₁ and/or R₂ may be an alkyl, for example the hydrocarbon methyl. In further aspects of embodiments, X may be a Group 16 element, for example Oxygen. In other aspects of embodiments, Y and/or A may each be a Group 15 element, for example Nitrogen.

According to more aspects of embodiments, Z may deprotinated and/or protinated. In one aspect of embodiments, Z may be a deprotinated Group 15 element, for example a deprotinated Nitrogen. In another aspect of embodiments, Z may be bonded to an alkyl, alkylene, alkene, aryl, heteroaryl, halogen, hydrogen, hydroxyl and/or the like, and combinations thereof. Referring back to Table 1, an HIV viral inhibitor may include 6-bromo-5-methyl-1H-indole-2,3-dione 3-[(6-bromo-5-methyl-2-oxo-1,2-dihydro-3H-indol-3-ylidene)hydrazone] (BIOder, compound 6).

According to some aspects of embodiments, Z may be a protonated Group 16 element, for example a protonated Oxygen (e.g., OH). Referring back to Table 1, an HIV viral inhibitor may include 4-bromo-5-methyl-1H-indole-2,3-dione 3-oxime (compound 4). In another aspect of embodiments, an HIV viral inhibitor may include 6-chloro-7-methyl-1H-indole-2,3-dione 3-oxime (i.e., compound 18).

According to some aspects of embodiment, an HIV viral inhibitor may be operable as a GSK inhibitor, for example a GSK-3-β inhibitor. In other aspects of embodiments, an HIV viral inhibitor may be operable as a Tat-dependent transcription inhibitor. In one aspect of embodiments, an HIV viral inhibitor may be relatively potent, for example exhibiting an IC₅₀ of less than approximately 30 nM. In another aspect of embodiments, an HIV viral inhibitor may exhibit an IC₅₀ between approximately 0.03 nM and 0.5 nM. In embodiments, one or more viral inhibitors may be employed to minimize viral infection, neurological disease, for example minimize HAND, and/or the like.

According to some aspects of embodiments, an HIV viral activator compound may include a compound represented by the structure illustrated in example FIG. 3A.

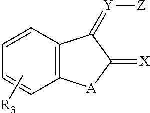

According to some aspects of embodiments, R₃ may include one or more alkyl, alkylene, alkene, aryl, heteroaryl, halogen, hydrogen, hydroxyl and/or the like, and combinations thereof. In one aspect of embodiments, R₃ may be a Group 17 element, for example Bromine. In another aspect of embodiments, R₃ may be an alkyl, for example the hydrocarbon methyl. In further aspects of embodiments, X may be a Group 16 element, for example Oxygen. In other aspects of embodiments, Y and/or A may each be a Group 15 element, for example Nitrogen.

According to more aspects of embodiments, Z may be protonated. In one aspect of embodiments, Z may be a protinated Group 15 element, for example a protinated Nitrogen. In another aspect of embodiments, Z may be bonded to an alkyl, alkylene, alkene, aryl, heteroaryl, halogen, hydrogen, hydroxyl and/or the like, and combinations thereof. In embodiments, Z may be NH—C(O)-benzyl. Referring back to Table 1, an HIV activator may include N′-(5-bromo-2-oxo-1,2-dihydro-3H-indol-3-ylidene)-2,4-dichlorobenzohydrazide (compound 16). In embodiments, an HIV viral activator may include N-(2-{[2-(5-bromo-2-oxo-1,2-dihydro-3H-indol-3-ylidene)hydrazino]carbonyl}phenyl)benzamide. (compound 31).

According to some aspects of embodiments, a VEEV viral inhibitor compound may include a compound represented by the structure illustrated in example FIG. 2A.

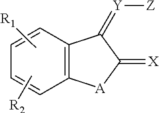

According to some aspects of embodiments, R₁ and/or R₂ may include one or more alkyl, alkylene, alkene, aryl, heteroaryl, halogen, hydrogen, hydroxyl and/or the like, and combinations thereof. In one aspect of embodiments, R₁ and R₂ may be on adjacent ring positions, for example positions 4 and 5, 5 and 6 and/or 6 and 7 on an inidole. In another aspect of embodiments, R₁ and/or R₂ may be a Group 17 element, for example Bromine, and the other of R₁ and/or R₂ may be an alkyl, for example the hydrocarbon methyl. In further aspects of embodiments, X may be a Group 16 element, for example Oxygen. In other aspects of embodiments, Y and/or A may each be a Group 15 element, for example Nitrogen.

According to some aspects of embodiments, Z may be a deprotinated Group 15 element, for example a deprotinated Nitrogen. In one aspect of embodiments, Z may be bonded to an alkyl, alkylene, alkene, aryl, heteroaryl, halogen, hydrogen, hydroxyl and/or the like, and combinations thereof. Referring back to Table 1, a VEEV viral inhibitor may include 6-bromo-5-methyl-1H-indole-2,3-dione 3-[(6-bromo-5-methyl-2-oxo-1,2-dihydro-3H-indol-3-ylidene)hydrazone] (BIOder, compound 6).

According to some aspects of embodiment, a VEEV viral inhibitor may be operable as a GSK inhibitor, for example a GSK-3-β inhibitor. In one aspect of embodiments, a VEEV viral inhibitor may be relatively potent, for example exhibiting an IC₅₀ of approximately 0.5 μM. In another aspect of embodiments, a VEEV viral inhibitor may exhibit a CC₅₀ of greater than approximately 100 μM. In further aspects of embodiments, a VEEV viral modulator may modulate the expression of a gene, for example modulate expression of a pro-apoptotic gene and/or an anti-apoptotic gene.

According to some aspects of embodiments, a VEEV viral inhibitor compound may include a compound represented by the structure illustrated in example FIG. 3A.

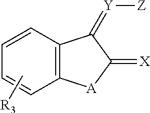

According to some aspects of embodiments, R₃ may include one or more alkyl, alkylene, alkene, aryl, heteroaryl, halogen, hydrogen, hydroxyl and/or the like, and combinations thereof. In one aspect of embodiments, R₃ may be a Group 17 element, for example Bromine. In another aspect of embodiments, X may be a Group 16 element, for example Oxygen. In further aspects of embodiments, Y and/or A may each be a Group 15 element, for example Nitrogen.

According to more aspects of embodiments, Z may be protonated. In one aspect of embodiments, Z may be a protinated Group 15 element, for example a protinated Nitrogen. In another aspect of embodiments, Z may be bonded to an alkyl, alkylene, alkene, aryl, heteroaryl, halogen, hydrogen, hydroxyl and/or the like, and combinations thereof. In further aspects of embodiments, Z may be NH—C(O)-halobenzyl. Referring back to Table 1, a VEEV viral inhibitor may include N′-(5-bromo-2-oxo-1,2-dihydro-3H-indol-3-ylidene)-2-chlorobenzohydrazide (compound 8). In embodiments, an VEEV viral inhibitor may include N′-(5-bromo-2-oxo-1,2-dihydro-3H-indol-3-ylidene)-2,4-dichlorobenzohydrazide. (compound 16).

According to some aspects of embodiments, a VEEV viral inhibitor compound may include a compound represented by the structure illustrated in example FIG. 2A.

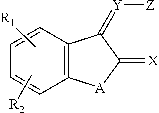

According to some aspects of embodiments, R₁ and/or R₂ may include one or more alkyl, alkylene, alkene, aryl, heteroaryl, halogen, hydrogen, hydroxyl and/or the like, and combinations thereof. In one aspect of embodiments, R₁ and R₂ may be on non-adjacent ring positions, for example positions 5 and 7 and/or 4 and 7 on an inidole. In another aspect of embodiments, R₁ and/or R₂ may be a Group 17 element, for example Bromine, and the other of R₁ and/or R₂ may be an alkyl, for example the hydrocarbon methyl. In further aspects of embodiments, X may be a Group 16 element, for example Oxygen. In other aspects of embodiments, Y and/or A may each be a Group 15 element, for example Nitrogen.

According to more aspects of embodiments, Z may be protonated. In one aspect of embodiments, Z may be a protinated Group 16 element, for example a protinated Oxygen (e.g., OH). Referring back to Table 1, a VEEV viral inhibitor may include 5,7-dibromo-1H-indole-2,3-dione 3-oxime (compound 10). In embodiments, a VEEV viral inhibitor may include 4-chloro-7-methyl-1H-indole-2,3-dione 3-oxime. (compound 19).

### 4. CONCLUSION

According to some aspects of embodiments, viral modulators may be determined and/or employed. In some aspects of embodiments, a viral modulator may regulate, inhibit, suppress, activate, up-regulate, and/or increase viral replication and/or effects. In one aspect of embodiments, a viral modulator may modulate cell viability and/or toxicity. In another aspect of embodiments, a viral modulator may be employed to address infections, diseases and/or the like. In other aspects of embodiments, a disease may include a neural disease, which may be induced by viral infection. In embodiments, diseases may include HAND, VEEV-related diseases. In embodiments, compounds may be employed and/or determined for neurological diseases, such as Alzheimer's, Parkinson's and/or the like.

According to some aspects of embodiments, HIV modulators may be determined and/or employed. BIO may be a relatively potent inhibitor of Tat-dependent transcription, for example compared to 3280 compounds screened. BIO, a synthetic derivative of the natural product, 6-bromoindirubin, may be a potent and/or specific GSK-3 inhibitor in HIV-related systems with an IC₅₀ of approximately 5 nM. BIO may also inhibit CDK5/p35, CDK2/Cyclin A, and/or CDK1/Cyclin B complexes with relatively higher IC₅₀s of approximately 0.08, 0.30 and/or 0.32 μM, respectively. Co-crystallization experiments may indicate that BIO may binds to the ATP pocket of GSK-3β, forming a van der Waals contact with Leu132, which is replaced by a phenylalanine in CDK2 and CDK5, providing one possible explanation for the preference of BIO for GSK-3β. BIO may be an effective GSK-3 inhibitor both in vitro and in vivo, through the accumulation of unphosphorylated β-catenin and/or through modulating Wnt signaling in Xenopus embryos.

There are other GSK-3 inhibitors including lithium, SB-216763, and SB-415286. Lithium may be active in the 10-20 mM range and/or may inhibit other molecules including polyphosphate-1-phosphate, inositol monophosphatase, casein kinase-II, MAP kinase-activated protein kinase-2 and/or p38-regulated/activated kinase. SB-216763 and SB-415286 may be identified as GSK-3α inhibitors through a high throughput screen of the SmithKline Beecham compound bank against rabbit GSK-3α, and/or may inhibit human GSK-3 with IC₅₀'s of approximately 34 nM and 78 nM, respectively. In one aspect of embodiments, BIO may exhibit a relatively lower IC₅₀ of all such GSK-3 inhibitors (approximately 5 nM), and therefore may have the most therapeutic potential.

According to some aspects of embodiments, BIOder may exhibit an in vitro IC₅₀ of approximately 0.03 nM and/or neuronal protection with relatively less toxicity than BIO. In some aspects of embodiments, one or more other kinases such as CK1, CLK1 and/or DYRK may be modulated by BIOder, which may jointly result in selective inhibition of a collection of other kinases and/or other targets in a cascade. BIOder may be efficacious in a variety of cells, for example U87MG, monocytes and/or macrophages with varying efficacy and/or potency.

According to some aspects of embodiments, GSK-3β may be employed in the treatment and/or suppression of inflammation. In some aspects of embodiments, GSK-3β may be important for both inflammation and cell migration. An upstream negative regulator of GSK-3β may be PI3K, which may limit the release of pro-inflammatory cytokines from monocytes and macrophages. In response to stimulation of Toll-like receptors in both monocytes and peripheral blood mononuclear cells, GSK-3β activity may be important for the production of pro-inflammatory cytokines, such as interleukin-6 (IL-6), IL-1β, tumor necrosis factor (TNF), and reduction of the anti-inflammatory cytokine IL-10. In terms of cell migration, GSK-3β inhibition may minimize extension of lamellipodia in keratinocytes and reduced axon elongation rates in neurons. Inhibition of GSK-3 through BIO treatment or RNAi may minimize migration of epithelial cells. Thus, GSK-3β inhibition through BIO treatment may have a profound effect on both inflammatory responses and cellular migration in response to inflammatory signals.

BIO may inhibit Tat-dependent transcription. GSK-3β may regulate a number of transcription factors and co-factors including β-catenin, c-Jun, c-Myc, C/EBPα/β, NFATc, RelA and CREB, most of which may be implicated in Tat mediated transcription. The β-catenin/T-cell factor (TCF) pathway may be of interest, which may have important connections in neuronal development and multiple neurological disorders. TCF may inhibit HIV transcription. While initial studies may indicate that the TCF mediated inhibition of HIV transcription may be β-catenin independent, later studies utilizing a TCF dominant negative construct, which is mutated in the β-catenin binding site, may indicate that β-catenin may be important for the observed effects. β-catenin binding to TCF may result in the release of TCF repressors, such as transducin-like enhancer, allowing the TCF/β-catenin complex to bind to DNA and regulate transcription. β-catenin proteasomal degradation may be induced by GSK-3β phosphorylation and thus stabilization of β-catenin may be expected following BIO treatment.

According to some aspects of embodiments, in addition to the β-catenin/TCF pathway, the NF-kB pathway may be relatively highly regulated by GSK-3β. Expression of a constitutively active GSK-3β mutant (S9A) and the inhibition of PI3K pathway (thus allowing GSK-3β to remain active) may result in astrocyte apoptosis. This may be due, at least in part, to the inhibition of the NF-kB pathway. In the presence of constitutively active GSK-3β, inhibition of NF-κB may be observed along with stabilization of the NF-κB-inhibitory protein, IκBα and down-regulation of IκB kinase (IKK) activity. GSK-3β may directly inhibit NF-kB through phosphorylation of RelA at serine 468, resulting in an inactivate form of NF-kB. However, the reverse may have also been demonstrated, where GSK-3β may be shown to be important for NF-κB mediated apoptosis in response to TNF-□treatment. Thus the influence of GSK-3β on NF-κB activity may be stimulus, cell type and/or promoter specific.

BIO and BIOder may inhibit both Tat-dependent transcription and neuronal cell death. The combined anti-proliferative and anti-inflammatory properties of BIO and BIOder may make them an attractive treatment, including in the control of HAND and other neurodegenerative disorders. The relatively enhanced potency and/or cytotoxicity may make BIO and/or BIOder attractive treatment compounds, for example for inhibition in HIV infections and/or neurological diseases.

According to some aspects of embodiments, VEEV modulators may be determined and/or employed. Alphaviruses, including Venezuelan Equine Encephalitis Virus (VEEV), may cause disease in both equine and humans that may exhibit overt encephalitis in a substantial percentage of cases. There may be no specific antiviral therapeutics for the treatment of VEEV and/or no FDA approved vaccine. Features of the host immune response and tissue-specific responses may contribute to fatal outcomes as well as the development of encephalitis. VEEV infection of mice, for example, may induce transcription of pro-inflammatory cytokines genes (e.g. IFN-α, IL-6, IL-12, iNOS and TNF-α) within approximately 6 h. GSK-3β may be a host protein that may modulate pro-inflammatory gene expression and/or may be a therapeutic target in neurodegenerative disorders such as Alzheimer's. Hence, inhibition of GSK-3β in the context of encephalitic viral infections may be useful in a neuroprotective capacity.

According to some aspects of embodiments, relatively small molecule GSK-3β modulators, for example inhibitors, may be determined for their ability to inhibit VEEV induced cell death and/or replication. Thirty-eight BIO analogs may be tested. BIOder may exhibit the relatively most potent inhibitition, with an IC₅₀ of approximately 0.5 μM and a CC₅₀ greater than approximately 100 μM. BIOder may be a relatively more potent inhibitor of GSK-3β than BIO, as demonstrated through in vitro kinase assays from uninfected and infected cells. Cells treated with BIOder may demonstrate a relative increase in the pro-apoptotic gene, survivin, and a decrease in the anti-apoptotic gene, BID, indicating that modulation of pro- and anti-apoptotic genes may contribute to the protective effect of a BIOder treatment. Finally, BIOder may exhibit great promise as a VEEV therapeutic by partially protecting mice from VEEV mortality. Our studies demonstrate the utility of GSK-3 β inhibitors for modulating viral infection, for example VEEV infection.

According to some aspects of embodiments, VEEV infection may occur in two distinct phases, a lymphotrophic phase, followed by a neurotrophic phase, both of which may be fairly well recapitulated in mouse models of disease. VEEV infection may spread from the site of inoculation (usually the footpad in mice) to the locally draining lymph node, causing viremia and disseminating to other lymphoid organs. Viremia may be followed by the neurotropic phase of the disease. Infection of olfactory neuroepithelium as well as brain capillary endothelial cells may allow the virus to enter the brain where VEEV infects neurons and glial cells. Neuronal damage, which may be contributed to both necrosis as well as apoptosis, may be an important aspect of the brain lesions of VEE infection in mice. Non-infected neurons may also be subject to bystander affects, as substantially no VEEV antigen may be found in a subset of dying neurons.

According to some aspects of embodiments, BIOder may be able to inhibit VEEV induced cell death. RT-PCR analysis may show up-regulation of survivin and/or downregulation of BID in BIOder treated VEEV infected U87MG cells, indicating that BIOder treatment and/or GSK-3β inhibition may alter apoptotic gene regulation. While there is no published literature documenting GSK-3β's alteration of survivin and BID in particular, these results are in agreement with role of GSK-3β in regulating apoptosis, specifically the intrinsic pathway. Lithium inhibition of GSK-3β may result in up-regulation of Bcl-2, the down-regulation of p53, and inhibition of the c-Jun N-terminal kinase (JNK) pathway. Another interesting link between GSK-3β and apoptosis involves the Bcl-2 family member, Mcl-1. Mcl-1 may be an anti-apoptotic protein whose degradation may be induced through sequential phosphorylation by JNK and GSK-3β. p53 dependent apoptosis may leverage GSK-3β, for example through GSK-3β's phosphorylation of the acetyltransferase Tip60. Tip60 acetylation of both p53 and histone H4 may be important for p53 dependent apoptosis and PUMA expression. In contrast, GSK-3β may protect against TNF induced cytotoxicity and death receptor mediated extrinsic apoptotic pathways. Therefore, the modulation of the apoptotic response by GSK-3β may be a balancing act that may be controlled by the targets of GSK-3β phosphorylation in a cell type dependent and context specific manner.

According to some aspect s of embodiments, there may be interest in understanding how VEEV enters and/or replicates within the brain since the central nervous system is an immune privileged site. A number of studies may indicate that VEEV infection alters the blood brain barrier (BBB), which may be composed of brain capillary endothelial cells. Both fully virulent and VEEV replicons may alter the BBB. VEEV infection may induce a number of host factors that may mediate inflammation as well as alterations to the BBB. For example, monocyte chemoattractant protein-1 (MCP-1), which my modulate the BBB potentially through causing alteration of tight junction proteins in endothelial cells, may be up-regulated in the brains of VEEV infected mice. In addition, matrix metalloproteinase-9 (MMP-9), which may help to maintain the BBB, and intercellular adhesion molecule-1 (ICMA-1), a molecular marker for BBB breakdown, may both be up-regulated following VEEV infection. Treatment of VEEV-infected mice with a relatively small molecule compound inhibitor of MMP-9, GM6001 may delay the opening of the BBB as well as the mean time to death. These results indicate that minimizing access to the brain may not completely prevent VEEV pathogenesis. Inhibition of GSK3 in cultured brain microvascular endothelial cells may suppress the production of multiple inflammatory molecules and monocytes migration across cytokine-stimulated cells. In addition, inhibition of GSK3 in vivo may reduce leukocyte adhesion to brain endothelium under inflammatory conditions, indicating that GSK3 may promote stabilization of the BBB. It is possible that BIOder may, without being bound to any particular theory, be influencing the BBB, which coupled with the ability of BIOder to inhibit viral replication, may make GSK3 inhibitors such as BIOder promising therapeutic candidates to protect against VEEV induced mortality.

According to some aspects of embodiments, BIOder may be able to relatively decrease VEEV induced cell death, which is in line with well documented effects of GSK-3β inhibitors. Bioder may relatively decrease VEEV replication. Without being bound to any particular theory, it is possible that VEEV may employ host factors, including GSK-3β, as a part of its survival and replication strategy. The modulation of enzyme profile in the infected tissue may induce damage to the tissue due to the secretion of cytokines that may be under GSK-3β control. GSK-3β has numerous substrates, only some of which may be biologically relevant. Interestingly, many VEEV proteins contain the GSK-3 consensus phosphorylation motif Ser/ThrXXXSer/Thr, as determined using PhosphoMotif Finder www[dot]hprd[dot]org/PhosphoMotif_finder. Multiple GSK-3 phosphorylation motifs may be found within substantially all the non-structural proteins as well as the E2 and E1 proteins.

In this specification, “a” and “an” and similar phrase are to be interpreted as “at least one” and “one or more.” References to “an” embodiment in this disclosure are not necessarily to the same embodiment.

The disclosure of this patent document may incorporate material which is subject to copyright protection. The copyright owner has no objection to the facsimile reproduction by anyone of the patent document or the patent disclosure, as it appears in the Patent and Trademark Office patent file or records, for the limited purposes required by law, but otherwise reserves all copyright rights whatsoever.

While various embodiments have been described above, it should be understood that they have been presented by way of example, and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. Thus, the present embodiments should not be limited by any of the above-described exemplary embodiments.

As one non-limiting example, it should be noted that the above explanation has focused on the example(s) such as modulators including inhibitors and/or activators. However, one skilled in the art will recognize that embodiments of the invention could include any effect including suppression, blocking, amplifying, dampening and/or regulating. As another non-limiting example, one skilled in the art would recognize that units and/or measurement described herein are intended to be approximations, and where not expressly stated as an approximation are intended to be for illustrative purposes only. In a further example, one skilled in the art will recognize from review of the compounds that they may be employed to determine efficacy and/or potency, and/or in the treatment, for any neurological disorder, such as Parkinson's disease, Alzheimer's disease and/or the like. As a final example, one skilled in the are will recognize that VEEV activators may be demonstrated, for example including the general structure illustrated in FIG. 1 and identified as compounds 4, 21 and 38 of FIG. 11.

In addition, it should be understood that any figures that highlight any functionality and/or advantages, are presented for example purposes only. The disclosed architecture is sufficiently flexible and configurable, such that it may be utilized in ways other than that shown. For example, the steps described and/or the data flow listed in any figures may be re-ordered or only optionally used in some embodiments.

Further, the purpose of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

Finally, it is the applicant's intent that only claims that include the express language “means for” or “step for” be interpreted under 35 U.S.C. 112, paragraph 6. Claims that do not expressly include the phrase “means for” or “step for” are not to be interpreted under 35 U.S.C. 112, paragraph 6.

1) A method comprising contacting at least one biological system with at least one viral modulator, the biological system configured to be infected by at least one HIV virus, wherein at least one of the at least one viral modulator comprises at least one HIV viral inhibitor compound including the following structure; 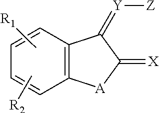
wherein,
R₁ and R₂ are on adjacent ring positions;
R₁ is a Group 17 element;
R₂ is a hydrocarbon;
X is a Group 16 element;
Y and A are each a Group 15 element; and
Z is one of:
(a) a deprotinated Group 15 element; or
(b) a protonated Group 16 element.

2) The method of claim 1, wherein:
a) the at least one biological system is a subject in need thereof; and
b) the contacting comprises administering a therapeutically effective amount of the at least one HIV viral inhibitor compound to the subject.

3) The method of claim 1, wherein the biological system comprises a cell.

4) The method of claim 3, wherein the cell is a neural cell.

5) The method of claim 1, wherein the biological system comprises a nervous system.

6) The method of claim 1, wherein the biological system comprises an in vitro system.

7) The method of claim 1, wherein the in vitro system comprises at least one of:
a) an assay system; and
b) a screen system.

8) The method of claim 1, wherein the at least one viral modulator compound comprises at least one of the following:
a) 4-bromo-5-methyl-1H-indole-2,3-dione 3-oxime;
b) 6-bromo-5-methyl-1H-indole-2,3-dione 3-[(6-bromo-5-methyl-2-oxo-1,2-dihydro-3H-indol-3-ylidene)hydrazone]; and
c) 6-chloro-7-methyl-1H-indole-2,3-dione 3-oxime.

9) The method of claim 1, wherein the at least one viral modulator compound comprises 4-bromo-5-methyl-1H-indole-2,3-dione 3-oxime.

10) The method of claim 9, wherein the at least one viral modulator compound comprises an IC₅₀ of less than approximately 30 nM.

11) The method of claim 9, wherein the at least one viral modulator compound comprises an IC₅₀ between approximately 0.03 nM and 0.5 nM.

12) The method of claim 1, wherein the at least one viral modulator compound is a GSK-3-β inhibitor.

13) The method of claim 1, wherein the at least one viral modulator compound is a Tat-dependent transcription inhibitor.

14) A method comprising contacting at least one biological system with at least one viral modulator, the biological system configured to be infected by at least one HIV virus, wherein at least one of the at least one viral modulator comprises at least one HIV viral activator compound including the following structure; 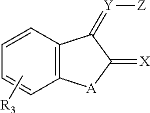
wherein,
R₃ is one of:
(a) a group 17 element; or
(b) a hydrocarbon;
X is a Group 16 element;
Y and A are each a Group 15 element; and
Z is NH—C(O)-benzyl.

15) The method of claim 14, wherein the at least one viral modulator compound comprises at least one of the following:
a) N′-(5-bromo-2-oxo-1,2-dihydro-3H-indol-3-ylidene)-2,4-dichlorobenzohydrazide; and
b) N-(2-{[2-(5-bromo-2-oxo-1,2-dihydro-3H-indol-3-ylidene)hydrazino]carbonyl}phenyl)benzamide.

16) A method comprising:
a) transducing a JLTRG cell with a Tat expression vector under the control of a murine stem cell promoter; and
b) isolating an eGFP-expressing cell from the JLTRG transduced cells.

17) The method of claim 16, further comprising at least one additional transduction on the isolated eGFP-expressing cell employing the Tat expression vector.

18) The method of claim 16, further comprising transducing the isolated eGFP-expressing cell employing an RFP-expressing vector.

19) The method of claim 18, further comprising single cell cloning for eGFP/RFP expressing cells to isolate a TiGR cell.

20) The method of claim 19, further comprising contacting the isolated TiGR cell with at least one compound to determine Tat modulation.


SEQUENCE LISTING (21 sequences)

SEQ ID NO: 1 - DNA, 22 bp, Artificial Sequence
  primer
tctgacaagacgttcccaatca

SEQ ID NO: 2 - DNA, 22 bp, Artificial Sequence
  primer
gaataacttccctccgaccaca

SEQ ID NO: 3 - DNA, 28 bp, Artificial Sequence
  primer
tgttggaagggaagataaacggctacgc

SEQ ID NO: 4 - DNA, 22 bp, Artificial Sequence
  primer
aggaagtgaacatttcggtgac

SEQ ID NO: 5 - DNA, 19 bp, Artificial Sequence
  primer
gctcagttccaggaccagg

SEQ ID NO: 6 - DNA, 20 bp, Artificial Sequence
  primer
tttctcaaggaccaccgcat

SEQ ID NO: 7 - DNA, 22 bp, Artificial Sequence
  primer
ccagctccttgaagcagaagaa

SEQ ID NO: 8 - DNA, 22 bp, Artificial Sequence
  primer
tgggaagctcagtaactgggaa

SEQ ID NO: 9 - DNA, 22 bp, Artificial Sequence
  primer
gcatgtgtctgcatgctcagat

SEQ ID NO: 10 - DNA, 20 bp, Artificial Sequence
  primer
atggcagcagtaaagcaagc

SEQ ID NO: 11 - DNA, 24 bp, Artificial Sequence
  primer
cggaagagttcattcactacctgt

SEQ ID NO: 12 - DNA, 22 bp, Artificial Sequence
  primer
acactgtgaaccaggagtgagt

SEQ ID NO: 13 - DNA, 20 bp, Artificial Sequence
  primer
aacagctttggaggaagcca

SEQ ID NO: 14 - DNA, 20 bp, Artificial Sequence
  primer
tggtcaccttacctctgcaa

SEQ ID NO: 15 - DNA, 20 bp, Artificial Sequence
  primer
tcaaacaggctggtggcaat

SEQ ID NO: 16 - DNA, 20 bp, Artificial Sequence
  primer
tgcttcagggtttcatccag

SEQ ID NO: 17 - DNA, 18 bp, Artificial Sequence
  primer
ggcggcaatcatcctctg

SEQ ID NO: 18 - DNA, 19 bp, Artificial Sequence
  primer
aaccagcagcagccatcat

SEQ ID NO: 19 - DNA, 22 bp, Artificial Sequence
  primer
ccacaaactcgtcactcatcct

SEQ ID NO: 20 - DNA, 22 bp, Artificial Sequence
  primer
ggaaggtgaaggtcggagtcaa

SEQ ID NO: 21 - DNA, 20 bp, Artificial Sequence
  primer
ccttgacggtgccatggaat